(19) United States
(12) Patent Application Publication
(10) Pub. No.: US 2025/0099475 A1
(43) Pub. Date: Mar. 27, 2025

(54) METHODS OF TREATING HYPERTENSION BY PERIODIC SUPPRESSION OF ALDOSTERONE SYNTHASE

(71) Applicants: Mineralys Therapeutics, Inc., Radnor, PA (US); Mitsubishi Tanabe Pharma Corporation, Osaka (JP)

(72) Inventors: David Rodman, Radnor, PA (US); Brian Taylor Slingsby, Radnor, PA (US); Jon Congleton, Radnor, PA (US); Hidetoshi Shimizu, Osaka (JP); Yoshiyasu Ota, Osaka (JP); Madori Orihashi, Osaka (JP)

(73) Assignees: Mineralys Therapeutics, Inc., Radnor, PA (US); Mitsubishi Tanabe Pharma Corporation, Osaka (JP)

(21) Appl. No.: 18/730,540

(22) PCT Filed: Jan. 19, 2023
(86) PCT No.: PCT/IB2023/050444

§ 371 Date: Jul. 19, 2024

Related U.S. Application Data
(60) Provisional application No. 63/400,301, filed on Aug. 23, 2022.
(60) Provisional application No. 63/300,967, filed on Jan. 19, 2022.

(51) Int. Cl. A61K 31/53; A61P 9/12

(52) U.S. Cl. CPC A61K 31/53; A61P 9/12

(57) ABSTRACT

This invention provides method of treating hypertension in a hypertensive subject, the method comprising administering to the subject a CYP 11β2 beta hydroxylase inhibitor once or twice per day in an amount sufficient to inhibit 50% or more of CYP 11β2 beta hydroxylase's activity for 40-60% of a 24-hour period to thereby treat hypertension in the hypertensive subject.

This application claims the priority of U.S. Provisional Application No. 63/400,301, filed Aug. 23, 2022, and U.S. Provisional Application No. 63/300,967, filed Jan. 19, 2022, the contents of each of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### FIELD OF THE INVENTION

This invention relates to methods of treating hypertension by inhibiting aldosterone synthase (CYP 11β2 beta hydroxylase).

### BACKGROUND OF THE INVENTION

Aldosterone is the principal mineralocorticoid in humans, produced in the zona glomerulosa of the adrenal cortex by aldosterone synthase (CYP 11β2 beta hydroxylase). Aldosterone is a key component of the renin-angiotensin-aldosterone system (RAAS), acting primarily as a regulator of electrolyte and fluid homeostasis.

Mineralocorticoid receptor-blocking agents (mineralocorticoid receptor antagonists, MRA), such as spironolactone and eplerenone, prevent aldosterone from binding with the mineralocorticoid receptor. Several clinical studies have demonstrated their benefit in treating hypertension. Given the role aldosterone plays in RAAS, inhibition of aldosterone synthase represents a possible alternative to mineralocorticoid receptor-blocking agents for the treatment of hypertension. However, previous studies suggest that some of the effects of aldosterone may occur independently of stimulation of the mineralocorticoid receptor/classic steroid-receptor complex modulation (Grossmann, C., & Gekle, M., 2009; Good, D. W., 2007; Mihailidou, A. S., & Funder, J. W., 2005). In addition, mineralocorticoid receptors are not selective for aldosterone, instead having similar affinity for the glucocorticoids cortisol and corticosterone.

Still further, plasma aldosterone (PA) levels follow a circadian rhythm, with PA typically reaching a peak in the morning upon standing, and then gradually falling throughout the day (Williams, G. H. 1972). It is unknown to what extent the differing levels of PA throughout the day affect blood pressure in hypertensive subjects and it is unknown whether inhibition of aldosterone synthesis for part of the day can effectively reduce blood pressure in a hypertensive subject. In order to effectively treat hypertension, methods that inhibit aldosterone synthase in an appropriate amount and at an appropriate time are needed.

### SUMMARY OF THE INVENTION

This invention provides a method of treating hypertension in a hypertensive subject, the method comprising administering to the subject a CYP 11β2 beta hydroxylase inhibitor once or twice per day in an amount sufficient to inhibit 50% or more of CYP 11β2 beta hydroxylase's activity for 40-60% of a 24-hour period to thereby treat hypertension in the hypertensive subject.

This invention provides a method of treating hypertension in a hypertensive subject, the method comprising administering to the subject a CYP 11β2 beta hydroxylase inhibitor once or twice per day in an amount sufficient to reduce the serum aldosterone level of the subject by 50-90% relative to the subject's pre-drug level of serum aldosterone for a period not less than eight hours and not greater than 16 hours.

This invention provides a method of treating hypertension in a hypertensive subject, the method comprising administering to the subject a CYP 11β2 beta hydroxylase inhibitor once per day in an amount sufficient to inhibit 50% or more of CYP 11β2 beta hydroxylase's activity for between 1 and 16 hours, preferably for between 3 and 8 hours

This invention also provides a method of treating hypertension in a hypertensive subject, the method comprising administering to the hypertensive subject a CYP 11β2 beta hydroxylase inhibitor once or twice per day in an amount sufficient to lower the hypertensive subject's ambulatory systolic blood pressure by at least 10 mmHg relative to the hypertensive subject's ambulatory systolic blood pressure prior to administration of the CYP 11β2 beta hydroxylase inhibitor for a period of at least eight weeks.

This invention also provides a method of reducing a hypertensive subject's systolic blood pressure during sleep, the method comprising administering to the subject a CYP 11β2 beta hydroxylase inhibitor once or twice per day in an amount sufficient to reduce a hypertensive subject's systolic blood pressure during sleep

### BRIEF DESCRIPTION OF THE DRAWINGS

FIG. 1: Estimate of time above IC50 for various doses of Compound A HBr based on PKPD modeling from SAD study.

FIG. 2: Mean change in systolic blood pressure from baseline at week 4.

FIG. 3: Individual systolic blood pressure change from baseline in the 100 mg QD and 25 mg BID cohorts. Median values indicated by black horizontal bars on left and right of each chart.

FIG. 4: Boxplot of change in systolic blood pressure from baseline at week 4.

FIG. 5: Comparison of median serum potassium over time in placebo, 25 mg BID and 100 mg QD cohorts.

FIG. 6: Graph of all available data for serum K⁺ values in the placebo and 100 mg QD cohorts.

FIG. 7: Individual responses in automated office-measured blood pressure (AOBP), Serum K+, and estimated glomerular filtration rate (eGFR) in the 100 mg cohort. Median values indicated by black horizontal bars on left and right of each chart.

FIG. 8: Pharmacokinetic profile for single ascending dose administration by dose group (mean±SE). SE=standard error.

FIG. 9: Pharmacokinetic profile for multiple ascending dose administration by dose group and day (mean±SE, time is relative to last dose). SE=standard error.

FIG. 10: Aldosterone time profile for single ascending dose administration by dose group. Day −1 is the day prior to dosing, reflecting normal circadian rhythm. Day 1 is the day of dosing, showing suppression of plasma aldosterone (mean±SE). SE=standard error.

FIG. 11: Aldosterone time profile for multiple ascending dose administration by dose group. Day −1 is the day prior to initiating dosing, reflecting normal circadian rhythm. Day 7 is the final day of dosing, showing suppression of plasma aldosterone (mean±SE). SE=standard error.

FIG. 12: Aldosterone percent change from baseline time profile for multiple ascending dose administration by dose group on Day 7. (mean±SE). SE=standard error.

FIG. 13: Aldosterone percent change from baseline, time profile for single ascending dose administration by dose group.

FIG. 14: Individual pharmacokinetics and time course of aldosterone suppression and recovery in Part 1 single ascending dose.

FIG. 15: Effect of Compound A on aldosterone and cortisol in Part 1 single ascending dose. Aldosterone AUC₀₋₂₄ in Part 1 single ascending dose. AUC₀₋₂₄, area under the curve from 0 to 24 hours; AUC₀₋₇₂, area under the curve from 0 to 72 hours.

FIG. 16: Effect of Compound A on aldosterone and cortisol in Part 1 single ascending dose. Cortisol AUC₀₋₇₂ in Part 1 single ascending dose. AUC₀₋₂₄, area under the curve from 0 to 24 hours; AUC₀₋₇₂, area under the curve from 0 to 72 hours.

FIG. 17: Effect of Compound A on plasma renin activity in Part 2 multiple ascending dose.

FIG. 18: Effect of Compound A on 11-DOC in Part 2 multiple ascending dose.

FIG. 19: Effect of Compound A on renal sodium and potassium handling. Urine Na⁺ and log 10(Na⁺/K⁺) ratio.

FIG. 20: Effect of Compound A on renal sodium and potassium handling. Serum K⁺.

FIG. 21: Compound A HBr Study Schema. ABPM=ambulatory blood pressure monitoring; BP=blood pressure; BID=twice daily; EOT=end of treatment; FU=follow up; PRA=plasma renin activity; QD=once daily. ^{a}=If Screening results were available, inclusion/exclusion evaluation was performed. If subject was not eligible based on Screening results, they did not continue to Visit 4. If Screening results were not available, subject proceeded to Visit 4. If Screening results were not available at Visit 4, subject should attend Visit 5 to determine final eligibility. If eligible based on Screening results, ABPM assessment begins at Visit 5. ^{b}=The ABPM procedure initiated at home approximately 24 hours before Randomization (Study Day 1). Alternatively, sites were permitted choose to schedule an office visit on Study Day 0 (Visit 5) to initiate the ABPM procedure. Training for the ABPM procedure was done either at an office visit or via phone.

FIG. 22: Waterfall plots showing the AOBP change in systolic blood pressure at week 8. The figure shows waterfall plots of the full analysis and safety set (FAS) analysis of placebo, 50 mg QD and 100 mg QD groups and the per-protocol (PP) analysis of the 100 mg group. A modeled mean and per-protocol observed mean values are also shown for each group.

FIG. 23: Waterfall plots showing the AOBP change in systolic blood pressure at week 8. The figure shows waterfall plots of the FAS analysis of 12.5 mg QD, 12.5 mg BID, and 25 mg BID using all subjects with week 8 measurement. Modeled mean and per-protocol observed mean values are also shown for each group.

FIG. 24: A bar graph showing the mean change in systolic blood pressure from baseline. The figure provides a final analysis including both full analysis set (FAS, all evaluable subjects receiving at least one dose of Compound A HBr) and per-protocol (PP, only those receiving ≥75% of study drug with week 8 visit). Part 2 data shows the interim average of last visit week 5-6.

FIG. 25: Graph of mean observed automated office blood-pressure change from baseline at week 8 for QD dosing regimens, showing a dose-response to Compound A HBr. BID per-protocol cohorts are shown on the far right of the graph.

FIG. 26: Graph showing the systolic blood pressure change from baseline at week 8 for a 50 mg QD, 100 mg QD, 12.5 mg BID, and 25 mg BID pooled cohort, the lowest response quartile of the pooled cohorts, the highest response quartile of the pooled cohort, and placebo.

FIG. 27: A waterfall plot showing change in systolic blood pressure from placebo and 100 mg QD groups pooled from both Parts 1 and 2. Part 2 data from interim snapshot with all subjects randomized and average last visit week 5-6, minimum week 2.

FIG. 28: A graph showing the change in estimated glomerular filtration rate (eGFR) in different dosing cohorts.

FIG. 29: A graph showing an example of ambulatory 24-hour blood pressure monitoring. The graph shows the 24-hour ambulatory blood pressure (systolic) of a subject receiving Compound A HBr 100 mg QD versus baseline, showing an average 24-hour blood pressure reduction and restoration of normal nocturnal dipping pattern.

FIG. 30: A graph showing the change in systolic blood pressure at week 8 relative to baseline as measured using the ABPM full analysis set.

FIG. 31: Waterfall plots showing the 24-hour average and overnight average ABPM change at 8 weeks relative to baseline. 100 mg QD dose levels provide excellent 24-hour blood pressure reduction. Overnight blood pressure reduction from the 100 mg QD dose level appears to be superior to 25 mg BID.

### DETAILED DESCRIPTION OF THE INVENTION

### Methods of Treating Hypertension

This invention provides a method of treating hypertension in a hypertensive subject, the method comprising administering to the subject a CYP 11β2 beta hydroxylase inhibitor once or twice per day in an amount sufficient to inhibit 50% or more of CYP 11β2 beta hydroxylase's activity for 40-60% of a 24-hour period to thereby treat hypertension in the hypertensive subject.

In embodiments of the invention, 50% or more of CYP 11β2 beta hydroxylase's activity is inhibited for between 10 to 14 hours of a 24-hour period.

This invention provides a method of treating hypertension in a hypertensive subject, the method comprising administering to the subject a CYP 11β2 beta hydroxylase inhibitor once or twice per day in an amount sufficient to reduce the serum aldosterone level of the subject by 50-90% relative to the subject's pre-drug level of serum aldosterone for a period not less than eight hours and not greater than 16 hours.

In embodiments of the invention, the CYP 11β2 beta hydroxylase inhibitor reduces the serum aldosterone level of the subject by 60-80% relative to the subject's pre-drug level of serum aldosterone for a period not less than eight hours and not greater than 16 hours.

In embodiments of the invention, the CYP 11β2 beta hydroxylase inhibitor allows serum aldosterone of the subject to return to the subject's pre-drug level of serum aldosterone or greater during the period between 16 and 24 hours after the dose is administered.

This invention provides a method of treating hypertension in a hypertensive subject, the method comprising administering to the subject a CYP 11β2 beta hydroxylase inhibitor once per day in an amount sufficient to inhibit 50% or more of CYP 11β2 beta hydroxylase's activity for between 1 and 16 hours, preferably for between 3 and 8 hours.

This invention provides a method of treating hypertension in a hypertensive subject, the method comprising administering to the subject a CYP 11β2 beta hydroxylase inhibitor once per day in an amount sufficient to:
(a) inhibit 50% or more of CYP 11β2 beta hydroxylase's activity for between 1 and 16 hours, preferably for between 3 and 8 hours;
(b) inhibit 60% or more of CYP 11β2 beta hydroxylase's activity for between 1 and 13 hours, preferably for between 2 and 6 hours;
(c) inhibit 70% or more of CYP 11β2 beta hydroxylase's activity for between 1 and 9 hours, preferably for between 2 and 5 hours;
(d) inhibit 80% or more of CYP 11β2 beta hydroxylase's activity for between 1 and 6 hours, preferably for between 1 and 3 hours; and/or
(e) inhibit 90% or more of CYP 11β2 beta hydroxylase's activity for between 0 and 3 hours, preferably for between 0 and 1 hour;
to thereby treat hypertension in the hypertensive subject.

This invention provides a method of treating hypertension in a hypertensive subject, the method comprising administering to the subject a CYP 11β2 beta hydroxylase inhibitor once per day in an amount sufficient to:
(a) inhibit 50% or more of CYP 11β2 beta hydroxylase's activity for between 1 and 20 hours, preferably for between 4 and 11 hours;
(b) inhibit 60% or more of CYP 11β2 beta hydroxylase's activity for between 1 and 17 hours, preferably for between 3 and 9 hours;
(c) inhibit 70% or more of CYP 11β2 beta hydroxylase's activity for between 1 and 15 hours, preferably for between 2.5 and 7 hours;
(d) inhibit 80% or more of CYP 11β2 beta hydroxylase's activity for between 1 and 10 hours, preferably for between 2 and 5 hours; and/or
(e) inhibit 90% or more of CYP 11β2 beta hydroxylase's activity for between 1 and 5 hours, preferably for between 0.5 and 2.5 hours;
to thereby treat hypertension in the hypertensive subject.

In embodiments of the invention, the hypertensive subject is taking or has taken a hypertension medication selected from a diuretic, an ACE inhibitor, an angiotensin receptor blocker, a calcium channel blocker, or a combination of two or more thereof.

In embodiments of the invention, the hypertensive subject is taking or has taken at least two of said hypertension medications.

In embodiments of the invention, the CYP 11β2 beta hydroxylase inhibitor is administered to the subject once per day.

In embodiments of the invention, the amount of the CYP 11β2 beta hydroxylase inhibitor is administered in the morning.

In embodiments of the invention, the amount of the CYP 11β2 beta hydroxylase inhibitor is administered once per day in the morning.

In embodiments of the invention, the CYP 11β2 beta hydroxylase inhibitor is administered to the subject twice per day.

In embodiments of the invention, the CYP 11β2 beta hydroxylase inhibitor:
(a) is administered daily for at least one week;
(b) is administered daily for at least two weeks;
(c) is administered daily for at least four weeks; or
(d) is administered daily for at least eight weeks.

In embodiments of the invention, the CYP 11β2 beta hydroxylase inhibitor is selective for inhibition of CYP 11β2 beta hydroxylase activity relative to inhibition of CYP 11 β1 beta hydroxylase activity, preferably wherein the inhibition constant (Ki) for CYP 11 β1 beta hydroxylase divided by the Ki for CYP 11 β2 beta hydroxylase is greater than 100.

In embodiments of the invention, the CYP 11β2 beta hydroxylase inhibitor is administered to the hypertensive subject in an amount below the amount which causes the subject's serum and/or plasma 11-deoxycortisterone (11-DOC) levels to exceed 600 pmol/L, preferably below the amount which causes the subject's serum and/or plasma 11-DOC levels to exceed 400 pmol/L.

In embodiments of the invention, the CYP 11β2 beta hydroxylase inhibitor is administered to the hypertensive subject in an amount below the amount which causes an accumulation of 11-DOC above 0.1 ng/ml in the subject.

In embodiments of the invention, the CYP 11β2 beta hydroxylase inhibitor is administered to the hypertensive subject in an amount which does not cause a clinically meaningful upregulation of the subject's adrenocortical hormone synthesis.

In embodiments of the invention, the administration of the CYP 11β2 beta hydroxylase inhibitor is administered to the hypertensive subject in an amount which:
(a) does not cause a clinically meaningful reduction of the subject's serum and/or plasma cortisol levels, relative to the subject's serum and/or plasma cortisol levels prior to administration of the CYP 11β2 beta hydroxylase inhibitor;
(b) does not cause a clinically meaningful increase in the subject's serum and/or plasma 11-DOC levels relative to the subject's serum and/or plasma 11-DOC levels prior to administration of the CYP 11β2 beta hydroxylase inhibitor; and/or

In embodiments of the invention, does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor.

In embodiments, the CYP 11β2 beta hydroxylase inhibitor is administered to the hypertensive subject in an amount:
(a) which does not cause a reduction of more than 20% in the subject's serum and/or plasma cortisol levels, relative to the subject's serum and/or plasma cortisol levels prior to administration of the CYP 11β2 beta hydroxylase inhibitor, preferably which does not cause a reduction of more than 10% in the subject's serum and/or plasma cortisol levels, relative to the subject's serum and/or plasma cortisol levels prior to administration of the CYP 11β2 beta hydroxylase inhibitor;
(b) which does not cause an increase of more than 20% in the subject's serum and/or plasma 11-DOC levels relative to the subject's serum and/or plasma 11-DOC levels prior to administration of the CYP 11β2 beta hydroxylase inhibitor, preferably which does not cause an increase of more than 10% in the subject's serum and/or plasma 11-DOC levels relative to the subject's serum and/or plasma 11-DOC levels prior to administration of the CYP 11β2 beta hydroxylase inhibitor; and/or
(c) which does not cause an increase of more than 20% in the subject's serum and/or plasma 11-deoxycortisol levels relative to the subject's serum and/or plasma 11-deoxycortisol levels prior to administration of the CYP 11β2 beta hydroxylase inhibitor, preferably which does not cause an increase of more than 10% in the subject's serum and/or plasma 11-deoxycortisol levels relative to the subject's serum and/or plasma 11-deoxycortisol levels prior to administration of the CYP 11β2 beta hydroxylase inhibitor.

In embodiments of the invention, the CYP 11β2 beta hydroxylase inhibitor is a compound described in U.S. Pat. No. 10,029,993, the disclosure of which is incorporated by reference herein. In embodiments, the CYP11β2 beta hydroxylase inhibitor is a compound described in U.S. Pat. No. 10,329,263, the disclosure of which is incorporated by reference herein. In embodiments, the CYP11β2 beta hydroxylase inhibitor is a 1,2,4-triazine compound or a pharmaceutically acceptable salt thereof.

In embodiments, the CYP11β2 beta hydroxylase inhibitor is a compound of Formula (A) or a pharmaceutically acceptable salt thereof:

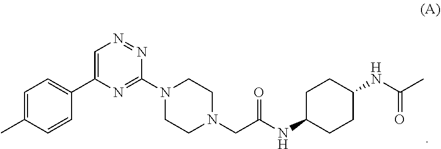 [SMILES: CC(=O)N[C@H]1CC[C@H](NC(=O)CN2CCN(c3nncc(-c4ccc(C)cc4)n3)CC2)CC1]

In embodiments, the CYP11β2 beta hydroxylase inhibitor is a pharmaceutically acceptable salt of the compound of Formula (A).

In embodiments, the CYP11β2 beta hydroxylase inhibitor is a monohydrobromide salt of the compound of Formula (A), i.e. Compound A HBr.

In embodiments, the CYP11β2 beta hydroxylase inhibitor is the free base form of the compound of Formula (A).

In embodiments:
(a) between 5 mg and 100 mg of the CYP 11β2 beta hydroxylase inhibitor is administered orally twice a day, 12 hours apart;
(b) between 10 mg and 50 mg of the CYP 11β2 beta hydroxylase inhibitor is administered orally twice a day, 12 hours apart;
(c) between 5 mg and 100 mg of the CYP 11β2 beta hydroxylase inhibitor is administered orally once a day; or
(d) between 10 mg and 50 mg of the CYP 11β2 beta hydroxylase inhibitor is administered orally once a day.

In embodiments:
(a) 12.5 mg of the CYP 11β2 beta hydroxylase inhibitor is administered orally twice a day, 12 hours apart;
(b) 25 mg of the CYP 11β2 beta hydroxylase inhibitor is administered orally twice a day, 12 hours apart;
(c) 12.5 mg of the CYP 11β2 beta hydroxylase inhibitor is administered orally once a day;
(d) 50 mg of the CYP 11β2 beta hydroxylase inhibitor is administered orally once a day; or
(e) 100 mg of the CYP 11β2 beta hydroxylase inhibitor is administered orally once a day.

In embodiments, the CYP11β2 beta hydroxylase inhibitor is a compound of formula (I) or a pharmaceutically acceptable salt thereof:

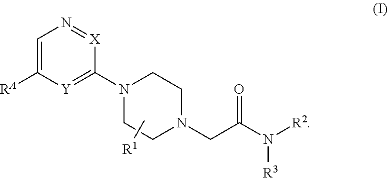

1) wherein X and Y represent any of the following (i) to (iii):
   (i) X is N, and Y is CH or C—RY,
   (ii) X is CH, and Y is N, or
   (iii) X is CH, and Y is CH;
2) R^{Y} represents an alkyl group;
3) R^{A} represents a cycloalkyl group which may be substituted, a cycloalkenyl group which may be substituted, an aryl group which may be substituted, or a 6- to 10-membered monocyclic or bicyclic heteroaryl group which may be partially hydrogenated and may be substituted;
4) R¹ represents a hydrogen atom, or an alkyl group;
5) R² represents an alkyl group which may be substituted, a cycloalkyl group which may be substituted, an aliphatic heterocyclic group which may be substituted, or a heteroaryl group which may be partially hydrogenated and may be substituted; and
6) R³ represents a hydrogen atom, or an alkyl group, or a pharmaceutically acceptable salt thereof.

In embodiments of the invention:
(a) the subject's office-measured systolic blood pressure is lowered relative to the subject's office-measured systolic blood pressure prior to administration of the CYP 11β2 beta hydroxylase inhibitor; and/or
(b) the subject's 24-hour ambulatory systolic blood pressure is lowered relative to the subject's ambulatory systolic blood pressure prior to administration of the CYP 11β2 beta hydroxylase inhibitor.

In embodiments of the invention:
(a) the subject's office-measured systolic blood pressure is lowered by at least 10 mmHg relative to the subject's office-measured systolic blood pressure prior to administration of the CYP 11β2 beta hydroxylase inhibitor; and/or
(b) the subject's ambulatory systolic blood pressure is lowered by at least 10 mmHg relative to the subject's ambulatory systolic blood pressure prior to administration of the CYP 11β2 beta hydroxylase inhibitor.

In embodiments of the invention:
(a) the subject's office-measured diastolic blood pressure is lowered relative to the subject's office-measured diastolic blood pressure prior to administration of the CYP 11β2 beta hydroxylase inhibitor;
(b) the subject's office-measured systolic and diastolic blood pressure is lowered relative to the subject's office-measured systolic and diastolic blood pressure prior to administration of the CYP 11β2 beta hydroxylase inhibitor;
(c) the subject's ambulatory systolic and diastolic blood pressure is lowered relative to the subject's ambulatory systolic and diastolic blood pressure prior to administration of the CYP 11β2 beta hydroxylase inhibitor; and/or
(d) the subject's systolic blood pressure is reduced to less than 130 mmHg and/or the subject's diastolic blood pressure is reduced to less than 80 mmHg.

In embodiments of the invention:
(a) the subject's ambulatory systolic blood pressure is lowered by at least 10 mmHg, and the subject's ambulatory diastolic blood pressure is lowered by at least 5 mmHg each relative to the subject's ambulatory systolic and diastolic blood pressure, respectively, prior to administration of the CYP 11β2 beta hydroxylase inhibitor;
(b) the subject's office-measured systolic blood pressure is lowered by at least 10 mmHg and the subject's office-measured diastolic blood pressure is lowered by at least 5 mmHg, each relative to the subject's office-measured systolic and diastolic blood pressure, respectively, prior to administration of the CYP 11β2 beta hydroxylase inhibitor; and/or
(c) the subject's systolic blood pressure is reduced to less than 130 mmHg and/or the subject's diastolic blood pressure is reduced to less than 80 mmHg.

In embodiments of the invention, the hypertensive subject's systolic blood pressure during sleep is reduced.

In embodiments of the invention, the hypertensive subject's average systolic blood pressure during sleep is reduced:
(a) by at least 10%, by between 10% and 40%, by between 10% and 30%, or by between 10% and 20% relative to the hypertensive subject's average daytime systolic blood pressure;
(b) by at least 8 mmHg, by at least 10 mmHg, by between 8 and 55 mmHg, by between 10 and 45 mmHg, or by between 10 and 25 mmHg, relative to their average systolic blood pressure during sleep prior to receiving the CYP 11β2 beta hydroxylase inhibitor.

In embodiments of the invention, the duration of inhibition of CYP 11β2 beta hydroxylase activity is sufficient to maintain a state of sodium and volume depletion in the hypertensive subject;

In embodiments of the invention, the method does not produce a persistent state of hyperkalemia or mild non-anion gap metabolic acidosis in the hypertensive subject; and/or

In embodiments of the invention, the CYP 11β2 beta hydroxylase inhibitor does not substantially accumulate in the hypertensive subject, preferably wherein the lack of substantial accumulation of the CYP 11β2 beta hydroxylase inhibitor in the hypertensive subject allows for the hypertensive subject's aldosterone levels to return to pre-drug baseline within 24-48 hours of the CYP 11β2 beta hydroxylase inhibitor being administered, more preferably within 16-24 hours of the CYP 11β2 beta hydroxylase inhibitor being administered.

In embodiments of the invention, the hypertensive subject's potassium levels are generally maintained in a clinically normal range, preferably wherein the hypertensive subject's potassium levels are mildly elevated relative to the hypertensive subject's potassium levels prior to administration of the CYP 11β2 beta hydroxylase inhibitor, more preferably wherein the hypertensive subject's potassium levels are elevated by 0.35 mmol/L or less, more preferably wherein the hypertensive subject's potassium levels are maintained below a level of 5.5 mmol/L, more preferably wherein the hypertensive subject's potassium levels are maintained between 3.5 mEq/l to 5.1 mEq/l.

In embodiments of the invention, the CYP 11β2 beta hydroxylase inhibitor is administered to the hypertensive subject in an amount which:
(a) suppresses aldosterone production in the subject;
(b) increases serum and/or plasma potassium levels in the subject; and/or
(c) increases plasma renin activity (PRA) in the subject.

In embodiments of the invention,
(a) serum and/or plasma aldosterone AUC-24 is reduced in the subject by at least 25% relative to the aldosterone levels in the subject prior to administration of the CYP 11β2 beta hydroxylase inhibitor;
(b) serum and/or plasma potassium levels in the subject are increased by at least 0.2 mMol/L relative to the serum and/or plasma potassium levels in the subject prior to administration of the CYP 11β2 beta hydroxylase inhibitor; and/or
(c) PRA in the subject is increased by at least 5 ng/ml/hr relative to the PRA in the subject prior to administration of the CYP 11β2 beta hydroxylase inhibitor.

In embodiments of the invention, the hypertensive subject's aldosterone level follows a substantially normal circadian rhythm.

In embodiments of the invention, the hypertensive subject has a plasma renin activity less than or equal to 1 ng/mL/hour.

In embodiments of the invention, the hypertensive subject has a plasma renin activity less than or equal to 0.6 ng/mL/hour.

In embodiments of the invention, the hypertensive subject has a plasma renin activity less than or equal to 4 ng/mL/hour.

In embodiments of the invention, the hypertensive subject has a plasma renin activity less than or equal to 3 ng/mL/hour.

In embodiments of the invention, the hypertensive subject has a plasma renin activity less than or equal to 2 ng/mL/hour.

In embodiments of the invention, the hypertensive subject has a plasma aldosterone concentration of greater than or equal to 6 ng/dL as measured by an immunoassay.

In embodiments of the invention, the hypertensive subject has a plasma aldosterone concentration of greater than or equal to 1 ng/dL as measured by LC-MS.

In a preferred embodiment of the invention, the hypertensive subject has a plasma renin activity less than or equal to 1 ng/mL/hour and a plasma aldosterone concentration of greater than or equal to 6 ng/dL as measured by an immunoassay. In a preferred embodiment of the invention, the hypertensive subject has a plasma renin activity less than or equal to 1 ng/mL/hour and a plasma aldosterone concentration of greater than or equal to 1 ng/dL as measured by LC-MS. In a further preferred embodiment, this hypertensive subject is taking or has taken a hypertension medication selected from a diuretic, an ACE inhibitor, an angiotensin receptor blocker, a calcium channel blocker, or a combination of two or more thereof.

This invention also provides a method of treating hypertension in a hypertensive subject, the method comprising administering to the hypertensive subject a CYP 11β2 beta hydroxylase inhibitor once or twice per day in an amount sufficient to lower the hypertensive subject's ambulatory systolic blood pressure by at least 10 mmHg relative to the hypertensive subject's ambulatory systolic blood pressure prior to administration of the CYP 11β2 beta hydroxylase inhibitor.

In embodiments, the method lowers the hypertensive subject's ambulatory systolic blood pressure by at least 10 mmHg relative to the hypertensive subject's ambulatory systolic blood pressure prior to administration of the CYP 11β2 beta hydroxylase inhibitor for a period of at least eight weeks.

This invention also provides a method of reducing a hypertensive subject's systolic blood pressure during sleep, the method comprising administering to the subject a CYP 11β2 beta hydroxylase inhibitor once or twice per day in an amount sufficient to reduce a hypertensive subject's systolic blood pressure during sleep.

In embodiments of the invention, the hypertensive subject's average systolic blood pressure during sleep is reduced:
(a) by at least 10%, by between 10% and 40%, by between 10% and 30%, or by between 10% and 20% relative to the hypertensive subject's average daytime systolic blood pressure;
(b) by at least 8 mmHg, by at least 10 mmHg, by between 8 and 55 mmHg, by between 10 and 45 mmHg, or by between 10 and 25 mmHg, relative to their average systolic blood pressure during sleep prior to receiving the CYP 11β2 beta hydroxylase inhibitor.

In embodiments of the invention, the hypertensive subject is taking or has taken a hypertension medication selected from a diuretic, an ACE inhibitor, an angiotensin receptor blocker, a calcium channel blocker, or a combination of two or more thereof. In embodiments of the invention, the hypertensive subject is taking or has taken at least two of said hypertension medications.

In embodiments of the invention, 50% or more of CYP 11β2 beta hydroxylase's activity is inhibited for 40-60% of a 24-hour period.

In embodiments of the invention, 50% or more of CYP 11β2 beta hydroxylase's activity is inhibited for between 10 to 14 hours of a 24-hour period.

In embodiments of the invention, the CYP 11β2 beta hydroxylase inhibitor reduces the serum aldosterone level of the subject by 50-90% relative to the subject's pre-drug level of serum aldosterone for a period not less than eight hours and not greater than 16 hours.

In embodiments of the invention, the CYP 11β2 beta hydroxylase inhibitor reduces the serum aldosterone level of the subject by 60-80% relative to the subject's pre-drug level of serum aldosterone for a period not less than eight hours and not greater than 16 hours.

In embodiments of the invention, the CYP 11β2 beta hydroxylase inhibitor allows serum aldosterone of the subject to return to the subject's pre-drug level of serum aldosterone or greater during the period between 16 and 24 hours after the dose is administered.

In embodiments of the invention, 50% or more of CYP 11β2 beta hydroxylase's activity is inhibited for between 1 and 16 hours, or preferably for between 3 and 8 hours, of a 24-hour period.

In embodiments of the invention, the CYP 11β2 beta hydroxylase inhibitor is administered to the subject once per day. In embodiments of the invention, the CYP 11β2 beta hydroxylase inhibitor is administered in the morning. In embodiments of the invention, the CYP 11β2 beta hydroxylase inhibitor is administered to the subject twice per day.

In embodiments of the invention, the CYP 11β2 beta hydroxylase inhibitor:
(a) is administered daily for at least one week;
(b) is administered daily for at least two weeks;
(c) is administered daily for at least four weeks; or
(d) is administered daily for at least eight weeks.

In embodiments of the invention, the hypertensive subject's ambulatory systolic blood pressure is reduced by 10-55 mmHg, by 10-50 mmHg, by 10-45 mmHg, by 10-40 mmHg, by 10-35 mmHg, by 10-30 mmHg, by 10-25 mmHg, by 10-20 mmHg, or by 10-15 mmHg, relative to the hypertensive subject's ambulatory systolic blood pressure prior to administration of the CYP 11β2 beta hydroxylase inhibitor for a period of at least eight weeks. In embodiments of the invention, the hypertensive subject's ambulatory systolic blood pressure is reduced by 5-25 mmHg, by 5-20 mmgHg, or by 5-15 mmHg relative to the hypertensive subject's ambulatory diastolic blood pressure prior to administration of the CYP 11β2 beta hydroxylase inhibitor for a period of at least eight weeks.

In embodiments of the invention, the duration of inhibition of CYP 11β2 beta hydroxylase activity is sufficient to maintain a state of sodium and volume depletion in the hypertensive subject.

In embodiments of the invention, the method does not produce a persistent state of hyperkalemia or mild non-anion gap metabolic acidosis in the hypertensive subject.

In embodiments of the invention, the CYP 11β2 beta hydroxylase inhibitor does not substantially accumulate in the hypertensive subject, preferably wherein the lack of substantial accumulation of the CYP 11β2 beta hydroxylase inhibitor in the hypertensive subject allows for the hypertensive subject's aldosterone levels to return to pre-drug baseline within 24-48 hours of the CYP 11β2 beta hydroxylase inhibitor being administered, more preferably within 16-24 hours of the CYP 11β2 beta hydroxylase inhibitor being administered.

In embodiments of the invention, the hypertensive subject's potassium levels are generally maintained in a clinically normal range, preferably wherein the hypertensive subject's potassium levels are mildly elevated relative to the hypertensive subject's potassium levels prior to administration of the CYP 11β2 beta hydroxylase inhibitor, more preferably wherein the hypertensive subject's potassium levels are elevated by 0.35 mmol/L or less, more preferably wherein the hypertensive subject's potassium levels are maintained below a level of 5.5 mmol/L, more preferably wherein the hypertensive subject's potassium levels are maintained between 3.5 mEq/l to 5.1 mEq/l.

In embodiments of the invention, the CYP 11β2 beta hydroxylase inhibitor is administered to the hypertensive subject in an amount which:
(a) suppresses aldosterone production in the subject;
(b) increases serum and/or plasma potassium levels in the subject; and/or
(c) increases plasma renin activity (PRA) in the subject.

In embodiments of the invention:
(a) serum and/or plasma aldosterone AUC-24 is reduced in the subject by at least 25% relative to the aldosterone levels in the subject prior to administration of the CYP 11β2 beta hydroxylase inhibitor;
(b) serum and/or plasma potassium levels in the subject are increased by at least 0.2 mMol/L relative to the serum and/or plasma potassium levels in the subject prior to administration of the CYP 11β2 beta hydroxylase inhibitor; and/or
(c) PRA in the subject is increased by at least 5 ng/ml/hr relative to the PRA in the subject prior to administration of the CYP 11β2 beta hydroxylase inhibitor.

In embodiments of the invention, the hypertensive subject's aldosterone level follows a substantially normal circadian rhythm.

In embodiments of the invention, said CYP 11β2 beta hydroxylase inhibitor is selective for inhibition of CYP 11β2 beta hydroxylase activity relative to inhibition of CYP 11 β1 beta hydroxylase activity, preferably wherein the inhibition constant (Ki) for CYP 11 β1 beta hydroxylase divided by the Ki for CYP 11 β2 beta hydroxylase is greater than 100.

In embodiments of the invention, the CYP 11β2 beta hydroxylase inhibitor is administered to the hypertensive subject in an amount below the amount which causes the subject's serum and/or plasma 11-deoxycortisterone (11-DOC) levels to exceed 600 pmol/L, preferably below the amount which causes the subject's serum and/or plasma 11-DOC levels to exceed 400 pmol/L.

In embodiments of the invention, the CYP 11β2 beta hydroxylase inhibitor is administered to the hypertensive subject in an amount below the amount which causes an accumulation of 11-DOC above 0.1 ng/ml in the subject.

In embodiments of the invention, the CYP 11β2 beta hydroxylase inhibitor is administered to the hypertensive subject in an amount which does not cause a clinically meaningful upregulation of the subject's adrenocortical hormone synthesis.

In embodiments of the invention, the CYP 11β2 beta hydroxylase inhibitor is administered to the hypertensive subject in an amount which:
(a) does not cause a clinically meaningful reduction of the subject's serum and/or plasma cortisol levels, relative to the subject's serum and/or plasma cortisol levels prior to administration of the CYP 11β2 beta hydroxylase inhibitor;
(b) does not cause a clinically meaningful increase in the subject's serum and/or plasma 11-DOC levels relative to the subject's serum and/or plasma 11-DOC levels prior to administration of the CYP 11β2 beta hydroxylase inhibitor; and/or
(c) does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor.

In embodiments of the invention, the CYP 11β2 beta hydroxylase inhibitor is administered to the hypertensive subject in an amount:
(a) which does not cause a reduction of more than 20% in the subject's serum and/or plasma cortisol levels, relative to the subject's serum and/or plasma cortisol levels prior to administration of the CYP 11β2 beta hydroxylase inhibitor, preferably which does not cause a reduction of more than 10% in the subject's serum and/or plasma cortisol levels, relative to the subject's serum and/or plasma cortisol levels prior to administration of the CYP 11β2 beta hydroxylase inhibitor;
(b) which does not cause an increase of more than 20% in the subject's serum and/or plasma 11-DOC levels relative to the subject's serum and/or plasma 11-DOC levels prior to administration of the CYP 11β2 beta hydroxylase inhibitor, preferably which does not cause an increase of more than 10% in the subject's serum and/or plasma 11-DOC levels relative to the subject's serum and/or plasma 11-DOC levels prior to administration of the CYP 11β2 beta hydroxylase inhibitor; and/or
(c) which does not cause an increase of more than 20% in the subject's serum and/or plasma 11-deoxycortisol levels relative to the subject's serum and/or plasma 11-deoxycortisol levels prior to administration of the CYP 11β2 beta hydroxylase inhibitor, preferably which does not cause an increase of more than 10% in the subject's serum and/or plasma 11-deoxycortisol levels relative to the subject's serum and/or plasma 11-deoxycortisol levels prior to administration of the CYP 11β2 beta hydroxylase inhibitor.

In embodiments, said CYP 11β2 beta hydroxylase inhibitor is a compound of Formula (A) or a pharmaceutically acceptable salt thereof:

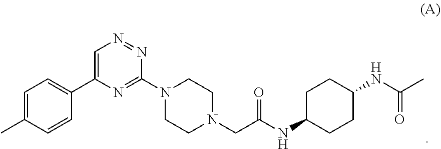 [SMILES: CC(=O)N[C@H]1CC[C@H](NC(=O)CN2CCN(c3nncc(-c4ccc(C)cc4)n3)CC2)CC1]

In embodiments, the compound is in the form of an HBr salt of the compound of Formula (A).

In embodiments:
(a) between 5 mg and 100 mg of the CYP 11β2 beta hydroxylase inhibitor is administered orally twice a day, 12 hours apart;
(b) between 10 mg and 50 mg of the CYP 11β2 beta hydroxylase inhibitor is administered orally twice a day, 12 hours apart;
(c) between 5 mg and 100 mg of the CYP 11β2 beta hydroxylase inhibitor is administered orally once a day; or
(d) between 10 mg and 50 mg of the CYP 11β2 beta hydroxylase inhibitor is administered orally once a day.

In embodiments:
(a) 12.5 mg of the CYP 11β2 beta hydroxylase inhibitor is administered orally twice a day, 12 hours apart;
(b) 25 mg of the CYP 11β2 beta hydroxylase inhibitor is administered orally twice a day, 12 hours apart;
(c) 12.5 mg of the CYP 11β2 beta hydroxylase inhibitor is administered orally once a day;
(d) 50 mg of the CYP 11β2 beta hydroxylase inhibitor is administered orally once a day; or
(e) 100 mg of the CYP 11β2 beta hydroxylase inhibitor is administered orally once a day.

In embodiments of the invention, the hypertensive subject has a plasma renin activity less than or equal to 1 ng/mL/hour.

In embodiments of the invention, the hypertensive subject has a plasma renin activity less than or equal to 0.6 ng/mL/hour.

In embodiments of the invention, the hypertensive subject has a plasma renin activity less than or equal to 4 ng/mL/hour.

In embodiments of the invention, the hypertensive subject has a plasma renin activity less than or equal to 3 ng/mL/hour.

In embodiments of the invention, the hypertensive subject has a plasma renin activity less than or equal to 2 ng/mL/hour.

In embodiments of the invention, the hypertensive subject has a plasma aldosterone concentration of greater than or equal to 6 ng/dL as measured by an immunoassay.

In embodiments of the invention, the hypertensive subject has a plasma aldosterone concentration of greater than or equal to 1 ng/dL as measured by LC-MS.

In embodiments of the invention, the hypertensive subject has secondary hypertension, preferably primary aldosteronism. In other embodiments of the invention, the hypertensive subject does not have primary aldosteronism, preferably wherein the hypertensive subject has primary hypertension.

In a preferred embodiment of the invention, the hypertensive subject has a plasma renin activity less than or equal to 1 ng/mL/hour and a plasma aldosterone concentration of greater than or equal to 6 ng/dL as measured by an immunoassay. In a preferred embodiment of the invention, the hypertensive subject has a plasma renin activity less than or equal to 1 ng/mL/hour and a plasma aldosterone concentration of greater than or equal to 1 ng/dL as measured by LC-MS. In a further preferred embodiment, this hypertensive subject is taking or has taken a hypertension medication selected from a diuretic, an ACE inhibitor, an angiotensin receptor blocker, a calcium channel blocker, or a combination of two or more thereof.

Alternatively, in embodiments wherein the hypertensive subject is not taking a hypertension medication selected from a diuretic, an ACE inhibitor, an angiotensin receptor blocker, a calcium channel blocker, in an embodiment the hypertensive subject has a plasma renin activity less than or equal to 0.6 ng/mL/hour and has a plasma aldosterone concentration of greater than or equal to 6 ng/dL as measured by an immunoassay or greater than or equal to 1 ng/dL as measured by LC-MS.

### Compositions

This invention also provides pharmaceutical compositions for use in any one of the methods described herein.

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

“Hypertension”, also called high blood pressure, is blood pressure that is higher than normal. In 2017, the American College of Cardiology and the American Heart Association published guidelines for hypertension management and defined hypertension as a blood pressure at or above 130 mmHg systolic blood pressure, 80 mmHg diastolic blood pressure. Stage 1 hypertension is defined as a blood pressure of 130-139 mmHg systolic blood pressure, 80-89 mmHg diastolic blood pressure, while stage 2 hypertension is defined as a blood pressure of greater than 140 mmHg systolic blood pressure, 90 mmHg diastolic blood pressure. As used herein, “hypertension” includes both stages 1 and 2 of hypertension unless indicated to the contrary. In an embodiment, the hypertensive subject has stage 1 hypertension. In another embodiment, the hypertensive subject has stage 2 hypertension. Hypertension includes high blood pressure that is multi-factorial and doesn't have one distinct cause (primary hypertension), and high blood pressure that has a direct cause (secondary hypertension). As used herein, “hypertension” includes both primary and secondary hypertension unless indicated to the contrary. In embodiments, the hypertensive subject has primary hypertension. In other embodiments, the hypertensive subject has secondary hypertension. Primary aldosteronism (hyperaldosteronism), the most common form of secondary hypertension, is a condition that occurs when the adrenal glands produce too much aldosterone. In embodiments where the hypertensive subject has secondary hypertension, the subject has primary aldosteronism.

“CYP11β2”, “Cyp11B2”, or “CYP11β2 beta hydroxylase” is a cytochrome P450 enzyme, encoded by the CYP11B2 gene in humans, which catalyzes a series of reactions leading from 11-deoxycorticosterone (i.e., an aldosterone precursor) to aldosterone. Thus, it is referred to in the art as “aldosterone synthase.” Cyp11B2 is mainly expressed in an adrenal cortex spherical layer and a level of plasma aldosterone is regulated by enzymatic activity of Cyp11B2 present in the adrenal gland. Aldosterone is expressed in other tissues, such as cardiovascular, kidney, adipose, and brain.

“CYP11β1”, “Cyp11B1” or “CYP11β1 beta hydroxylase” is a cytochrome P450 enzyme, encoded by the CYP11B1 gene in humans, which is involved in the biosynthesis of adrenal corticosteroids. It is referred to in the art as “steroid 11β-hydroxylase.”

An “inhibitor” refers to a compound (e.g. compounds described herein) that reduces activity when compared to a control, such as absence of the compound or a compound with known inactivity. An inhibitor can be a small molecule inhibitor, an antibody inhibitor, a protein inhibitor, a biomolecule inhibitor, a natural ligand, and the like. An “inhibitor” may be in the form of a pharmaceutically acceptable salt, e.g. of the compounds described herein.

As used herein “Compound A” refers to the disubstituted 1, 2, 4-Triazine compound which is represented by Formula (A):

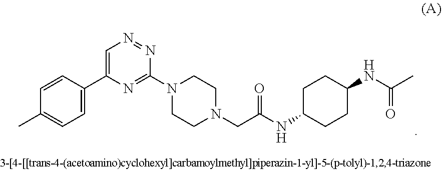 [SMILES: CC(=O)N[C@H]1CC[C@H](NC(=O)CN2CCN(c3nncc(-c4ccc(C)cc4)n3)CC2)CC1]

“Compound A HBr” refers to the hydrobromide (HBr) salt of Compound A. Weights and/or strengths of “Compound A HBr,” and “the compound” of the invention refer to the weight of the free base in the HBr salt (i.e. Compound A) and not the weight of the HBr salt.

Compound A and pharmaceutically acceptable salts thereof can be made by processes described, for example, in U.S. Pat. No. 10,029,993 and European Publication No. 3549935, the disclosures of which are incorporated by reference herein in their entirety.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may be a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration may be required. For example, the compositions are administered to the subject in an amount and for a duration sufficient to treat the patient. In embodiments, the treating or treatment is not prophylactic treatment.

A “diuretic” refers to a hypertension medication that increases the production of urine, thereby increasing the amount of water and salt eliminated from the body. The diuretic can be a carbonic anhydrase inhibitor, a loop diuretic, a potassium-sparing diuretic, a thiazide diuretic, or any other diuretic known in the art. Exemplary carbonic anhydrase inhibitors include acetazolamide, brinzolamide, dorzolamide, dichlorphenamide, ethoxaolamide, zoniamide, indisulam, and methazolamide. Exemplary loop diuretics include bumatenide, ethacrynic acid, torsemide, and furosemide. Exemplary potassium-sparing diuretics include epelerenone, triamterene, spironolactone, and amiloride. Exemplary thiazide diuretics include indapamide, hydrochlorothiazide, chlorthalidone, metolazone, methyclothiazide, chlorothiazide, methylclothiazide, metolazone, bendroflumethiazide, polythiazide, and hydroflumethiazide. Other diuretics include pamabrom and mannitol.

An “angiotensin-converting enzyme inhibitor” or “ACE inhibitor” refers to a hypertension medication that block angiotensin I from being converted to angiotensin II, thereby dilating blood vessels and lowering blood pressure. Exemplary ACE inhibitors include benazepril, zofenopril, perindopril, trandolapril, captopril, enalapril, lisinopril, and ramipril.

An “angiotensin receptor blocker” or “angiotensin II inhibitor” refers to a hypertension medication that blocks the receptor binding of angiotensin II, thereby dilating blood vessels and lowering blood pressure. Exemplary angiotensin receptor blockers include eprosartan, olmesartan, valsartan, candesartan, losartan, telmisartan, irbesartan, valsartan, and azilsartan medoxomil.

A “calcium channel blocker” refers to hypertension medication that can block calcium from entering the cells of the heart and arteries via a calcium channel, thereby lowering blood pressure. A calcium channel blocker can be a dihydropyridine calcium channel blocker, a phenylalkylamine calcium channel blocker, a benzothiazepine calcium channel blocker, a nonselective calcium channel blocker, or any other calcium channel blocker known in the art. Dihydropyridine calcium channel blockers include amoldipine, aranidipine, azelnidipine, bamidipine, benidipine, cilnidine, clevidipine, efonidipine, felodipine, isradipine, lacidipine, lercanidipine, manidipine, nicardipine, nifedipine, nilvadipine, nimodipine, nisoldipine, nitrendipine, and pranidipine. Phenylalkylamine calcium channel blockers include fendiline, gallipamil, and verapamil. Benzothiazepine calcium channel blockers include diltiazem. Nonselective calcium channel blockers include mibefradil, bepridil, flunarizine, fluspirilene, and fendiline. Other calcium channel blockers include gabapentin, pregabalin, and ziconotide.

A “normal circadian rhythm” for aldosterone level follows a diurnal pattern wherein the nadir is in the late evening and the peak is in the early morning, pre-arousal. In an embodiment, the hypertensive subject's aldosterone level follows a substantially normal circadian rhythm. In one example of such an embodiment, the CYP 11β2 beta hydroxylase inhibitor of the invention, when dosed once daily in the morning after waking, suppresses the abnormally elevated production of Aldosterone during waking hours. In the early evening suppression of Aldosterone production starts to wane and the normal increase in serum Aldosterone returns towards normal by dawn, as it would under normal circumstances. The circadian rhythm of aldosterone in normal subjects and subjects with primary aldosteronism is described in Kem, David C., et al. “Circadian rhythm of plasma aldosterone concentration in patients with primary aldosteronism.” The Journal of clinical investigation 52.9 (1973): 2272-2277, the contents of which are specifically incorporated by reference herein.

This invention provides methods that reduce a hypertensive subject's systolic blood pressure “during sleep”. In this context, “during sleep” refers to the period of sleep in the hypertensive subject's normal sleep/wake cycle. In other words, “during sleep” refers to the hypertensive subject's approximately seven to nine hours of sleep (typically at night) that occurs daily in between their approximately 15 to 17 hours of wakefulness and not to any short period of sleep that may occur outside of the sleep phase of the subject's normal sleep/wake cycle (i.e. naps). The blood pressure of non-hypertensive individuals typically dips during sleep, with approximately 10% to 15% lower blood pressure values during sleep relative to during wakefulness. In contrast, hypertensive subjects may experience a smaller dip in blood pressure during sleep or may not experience any dip in blood pressure at all. Thus, methods of the invention help hypertensive subjects restore the dip in blood pressure that normal, non-hypertensive subjects experience during sleep.

A subject's “pre-drug level” of serum aldosterone refers to the subject's level of serum aldosterone, at the same time of day, in the absence of being treated with the CYP 11β2 beta hydroxylase inhibitor. As discussed above, aldosterone level follows a diurnal pattern where the nadir is in the late evening and the peak is in the early morning, pre-arousal. Therefore, in embodiments where the dose of CYP 11β2 beta hydroxylase inhibitor reduces the serum aldosterone level of the subject by a certain percentage relative to their “pre-drug level”, the reduction in serum aldosterone is measured relative to the same subject's serum aldosterone level in the absence of administration of the CYP 11β2 beta hydroxylase inhibitor at the same time of day. For example, the subject's serum aldosterone level at 11 AM when administered a CYP 11β2 beta hydroxylase inhibitor would be measured relative to that same subject's serum aldosterone level at 11 AM prior to any administration of the CYP 11β2 beta hydroxylase inhibitor.

### General

For the foregoing embodiments, each embodiment disclosed herein is contemplated as being applicable to each of the other disclosed embodiment.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1

A randomized, double-blinded, placebo-controlled study was conducted in which 116 patients were randomized and 87 received Compound A HBr.

A single ascending dose (SAD) study was conducted with the following dosing amounts tested:
(a) 5 mg
(b) 10 mg
(c) 20 mg
(d) 50 mg
(e) 100 mg
(f) 200 mg
(g) 400 mg
(h) 800 mg.

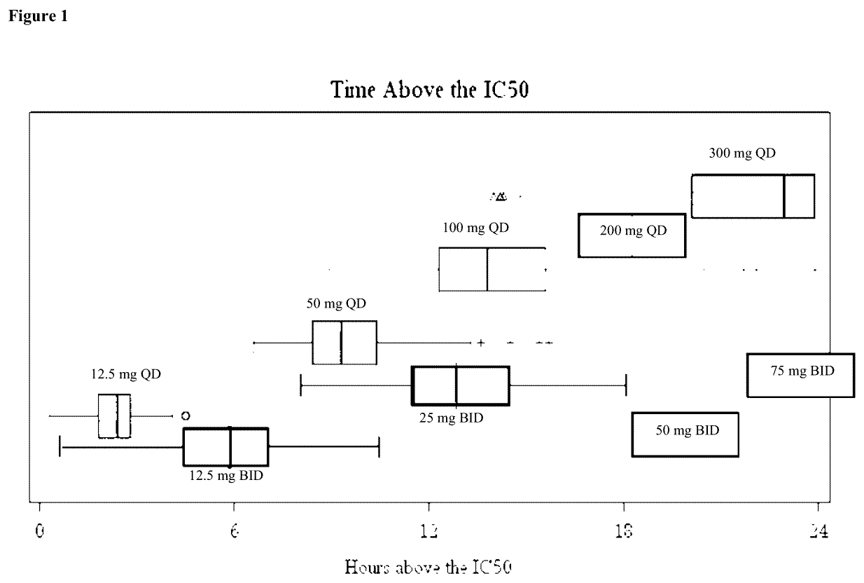

The IC50 of inhibition of CYP 11β2 beta hydroxylase by Compound A HBr and the time above IC50 for each dosing amount were estimated based on PKPD modeling of data from the SAD study. The estimated number of hours above IC50 and the proportion of a 24 hour period above IC50 for proposed dosing in the phase two, proof-of-concept study were extrapolated and are shown in the table below and in FIG. 1.

TABLE 1

Compound A HBr
Aldosterone

| | | |
|---|---|---|
| Cohort | Hours above IC50 | % 24 hr |

| | | | |
|---|---|---|---|
| 12.5 | mg QD | 2 | 8 |
| 50 | mg QD | 9 | 38 |
| 100 | mg QD | 13.5 | 56 |
| 12.5 | mg BID | 6 | 25 |
| 25 | mg BID | 12.5 | 52 |

Time above IC50 was closely correlated with the duration of aldosterone suppression in the SAD study.

In a Multiple Ascending Dose (MAD) Study, little drug accumulation was observed at 40 mg, 120 mg and 360 mg QD doses.

### Example 2

### Study Design

A double-blinded, randomized, placebo-controlled trial on the safety and efficacy of Compound A HBr was conducted on subjects with hypertension.

Hypertensive subjects were randomly assigned to one of the following six experimental groups:
(a) Placebo group: Oral administration of a placebo tablet matched for size, color, and shape of study drug once in the morning each day;
(b) 12.5 mg BID: Oral administration of one tablet containing 12.5 mg of Compound A HBr once in the morning and evening, 12 hours apart each day;
(c) 25 mg BID: Oral administration of one tablet containing 25 mg of Compound A HBr once in the morning and evening, 12 hours apart each day;
(d) 12.5 mg QD: Oral administration of one tablet containing 12.5 mg of Compound A HBr once in the morning each day;
(e) 50 mg QD: Oral administration of two tablets containing 25 mg of Compound A HBr once in the morning each day; and
(f) 100 mg QD: Oral administration of one tablet containing 100 mg of Compound A HBr once in the morning each day.

### Inclusion Criteria

Subjects in the study met the following inclusion criteria:
(a) Male and nonpregnant, nonlactating female subjects ≥18 years of age;
(b) Written informed consent Health Insurance Portability and Accountability Act authorization, and local patient privacy required documentation for this study have been obtained;
(c) Automated office blood pressure (AOBP) with SBP ≥130 mm Hg;
(d) Background antihypertensive treatment of ≥2 drugs;
(e) PRA ≤1.0 ng/mL;
(f) Serum aldosterone ≥1 ng/dL; and
(g) Morning serum cortisol in normal range. If baseline serum cortisol <18 mcg/dL, then baseline Cortrosyn stimulation test administered at baseline.

### Exclusion Criteria

Prospective subjects were also excluded based on the following exclusion criteria:
(a) Concomitant use of epithelial sodium channel inhibitors or mineralocorticoid receptor antagonists;
(b) Subjects with hypokalemia;
(c) Subjects with hyperkalemia;
(d) Subjects with serum cortisol <3 mcg/dL;
(e) Subjects with serum sodium <135 mEq/L;
(f) Subjects with estimated glomerular filtration rate <60 mL/min/1.73 m2;
(g) Subjects with type 1 or uncontrolled (hemoglobin A1c ≥9%) type 2 diabetes mellitus;
(h) Subjects with body mass index >40 kg/m2;
(i) Subjects with unstable angina;
(j) Subjects with SBP ≥175 mm Hg or DBP ≥100 mm Hg;
(k) Subjects with a decrease in SBP ≥20 mm Hg or DBP ≥10 mm Hg from sitting to standing position;
(l) Subjects who, in the opinion of the investigator, have suspected nonadherence to antihypertensive treatment;
(m) Subjects who, in the opinion of the investigator, have any major medical illness or symptoms;
(n) Subjects who, in the opinion of the investigator, have any acute or chronic medical or psychiatric condition;
(o) Subjects undergoing treatment with any of the following medications:
   (i) Topical corticoids;
   (ii) Sympathomimetic decongestants;
   (iii) Theophylline;
   (iv) Phosphodiesterase type 5 inhibitors;
   (v) NSAIDs;
   (vi) Intramuscular steroids;
   (vii) Estrogen;
   (viii) Cytochromes;
   (ix) Strong CYP3A and CYP3A4 inducers;
(p) Subjects with known hypersensitivity to Compound A HBr or any of the excipients; and
(q) Subjects who are night-shift workers.

### Results

Change in Systolic Blood Pressure from Baseline at Week 4

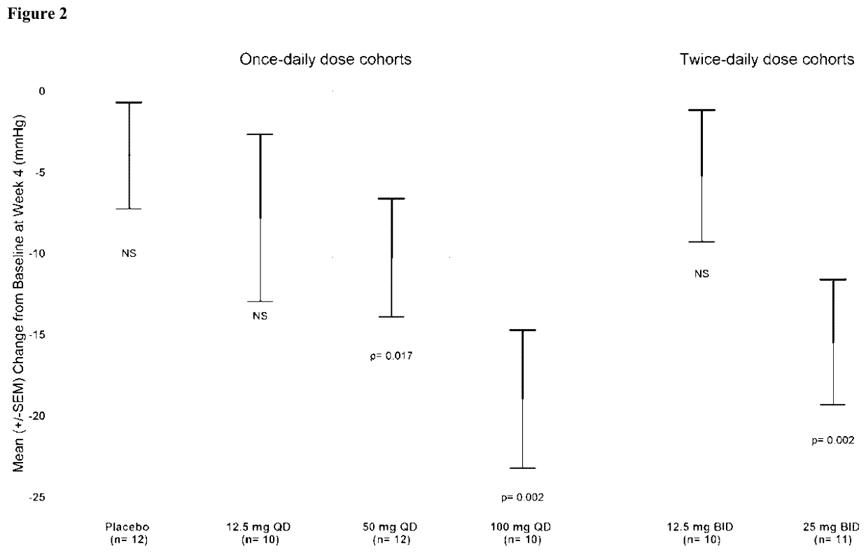

At week 4 of treatment, single daily doses of Compound A HBr demonstrated a dose-response with activity seen at 50 mg QD, increasing further at 100 mg QD. Placebo showed a small, non-significant reduction. Twice daily dosing at 25 mg BID was as effective as 100 mg QD. See FIG. 2.

A summary table of the within-cohort change in systolic blood pressure from baseline at week 4 is shown in the table below:

| All values | | | | | | | | |
|---|---|---|---|---|---|---|---|---|
| mmHg | | | | | | Change (Wk 4 | | Within cohort |
| measured by | | Baseline | | Week 4 | | v. BL) | | Wk 4 v. BL |
| AOBP | | Mean | | Mean | | Mean | | (p- |
| Cohort | N | (SEM) | Median | (SEM) | Median | (SEM) | Median | value) |
| Placebo | 12 | 145.5 | 145.25 | 141.5 | 143.5 | −4.0 | −6.75 | 0.248 |
| | | (2.2) | | (3.7) | | (3.3) | | |
| 12.5 mg QD | 10 | 146.8 | 147.75 | 138.9 | 139 | −7.9 | −2.25 | 0.161 |
| | | (2.5) | | (5.3) | | (5.1) | | |
| 50 mg QD | 12 | 142.4 | 139.25 | 132.1 | 133 | −10.3 | −8.75 | 0.017 |
| | | (2.5) | | (3.1) | | (3.6) | | |
| 100 mg QD | 10 | 145.1 | 143.25 | 126.1 | 124 | −19.0 | −20.25 | 0.002 |
| | | (3.2) | | (4.4) | | (4.3) | | |
| 12.5 mg BID | 10 | 143.2 | 141.75 | 138 | 133 | −5.3 | −4.5 | 0.228 |
| | | (3.6) | | (4.8) | | (4.1) | | |
| 25 mg BID | 11 | 147.6 | 147.5 | 132.1 | 132 | −15.5 | −20.0 | 0.003 |
| | | (2.5) | | (4.9) | | (3.9) | | |

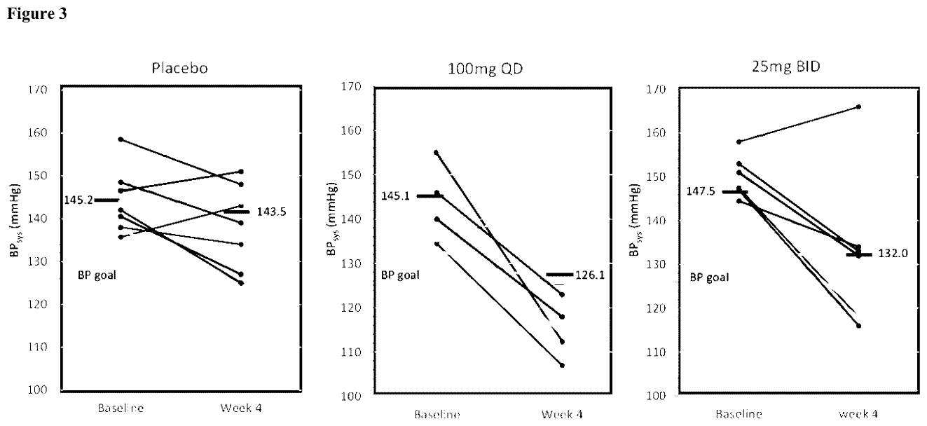

Individual systolic blood pressure change from baseline in the 100 mg QD and 25 mg BID cohorts is shown in FIG. 3. These cohorts had a high responder rate and consistent magnitude of benefit.

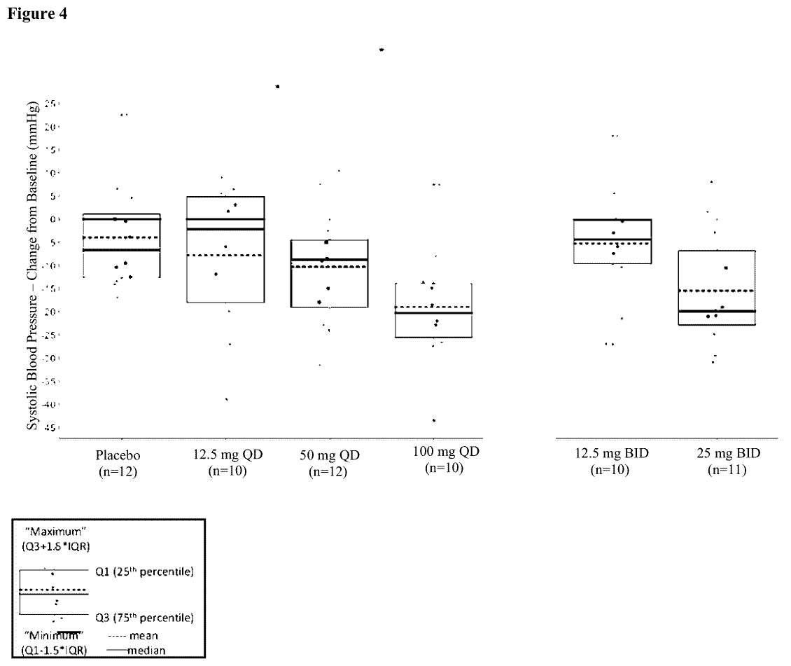

FIG. 4 provides a boxplot of the change in systolic blood pressure from baseline at week 4. Both the 100 mg QD and 25 mg BID dose levels produced a reduction in systolic blood pressure that was significantly greater than that seen in the placebo group. Formal inter-cohort comparisons will be completed at the end of the study using a mixed-effects model. The table below summarizes the results:

| | | |
|---|---|---|
| Dose Cohort | Mean (SEM) | P-value (vs Pbo) |

| | | | |
|---|---|---|---|
| 12.5 | mg QD | 3.85 (6.1) | 0.537 |
| 50 | mg QD | 6.3 (4.9) | 0.213 |
| 100 | mg QD | 15 (5.4) | 0.012 |
| 12.5 | mg BID | 1.2 (5.2) | 0.813 |
| 25 | mg BID | 11.5 (5.1) | 0.034 |

*Significant at 5% level, 2-sample, 2-tailed t-test comparing each dose cohort vs Placebo, with no correction for multiplicity

### Effect on Serum K⁺

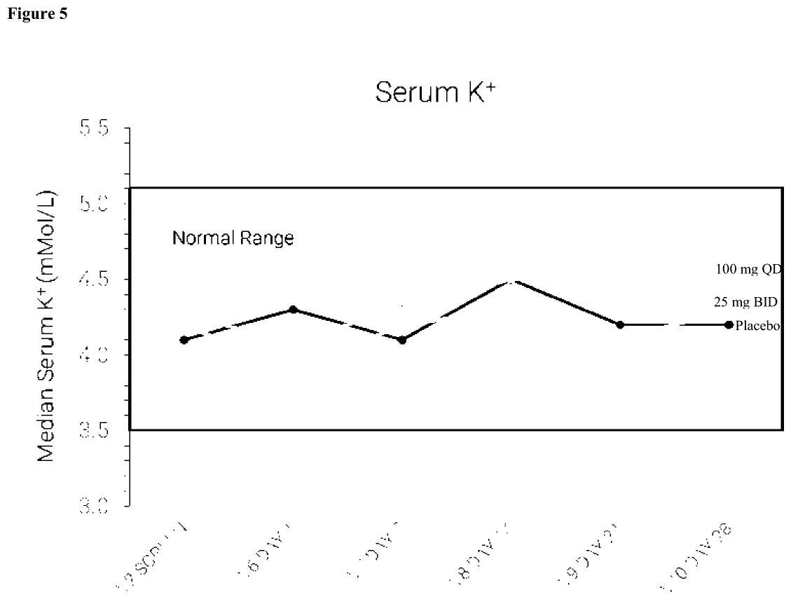

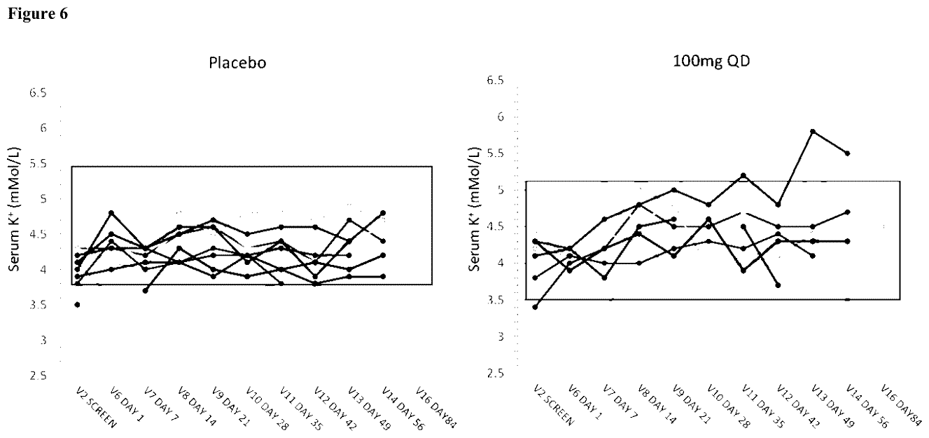

In the 100 mg QD cohort, a median increase of 0.2 mMol/L in median serum potassium (K⁺) was observed. See FIG. 5. No subjects in the 100 mg QD cohort had dose held or dose reduction related to elevated K⁺. Throughout the trial the majority of elevated serum K⁺ values were isolated events. Graphs showing all available data for serum K⁺ values in the placebo and 100 mg QD cohorts are provided in FIG. 6.

### Individual Responses in 100 mg Cohort

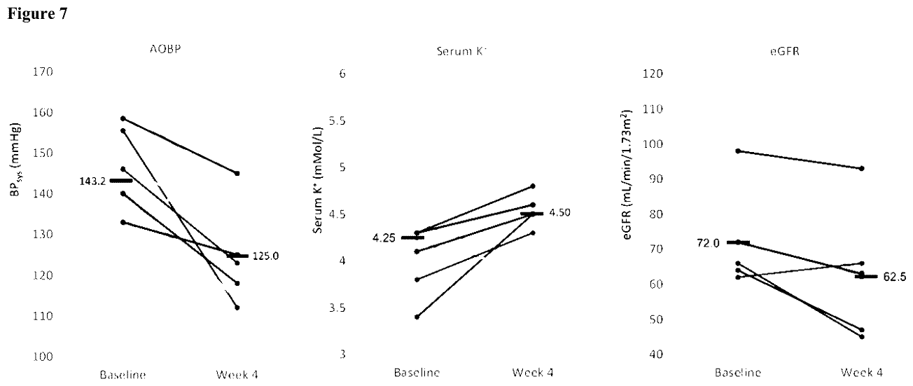

FIG. 7 shows all the individual responses in automated office-measured blood pressure (AOBP), Serum K⁺, and estimated glomerular filtration rate (eGFR) in the 100 mg cohort.

Reductions in BP were associated with on-target, modest increase in serum K+ and reduction in eGFR. Both the change in serum K⁺ and eGFR were monitorable and, in instances where Compound A HBr was held, dose-adjusted or discontinued, reversible.

### Discussion

The double-blinded, randomized, placebo-controlled trial described in Example 2 demonstrates that once daily administration of a 100 mg dose and twice daily administration of a 25 mg dose of Compound A HBr have the greatest effect on systolic blood pressure of all dosing regimens tested. See FIG. 2. Based on PKPD modeling of data from a SAD study as summarized in Example 1, it was determined that these dosing regimens provide, on average, a time above IC50 (for inhibition of CYP 11β2 beta hydroxylase) of about 12.5 to 13.5 hours per 24 hour period.

Without intending to be limited to a particular theory, the inventors hypothesize that methods which inhibit 50% or more of CYP 11β2 beta hydroxylase activity for between 40-60% of a 24-hour period, i.e. for about 10-14 hours per day, can be used to safely and effectively treat hypertension in a hypertensive subject. The results described in Example 2 demonstrate that this amount of inhibition of CYP 11β2 beta hydroxylase activity can reduce systolic blood pressure in a hypertensive subject without causing an adverse increase in serum potassium or other adverse reactions requiring cessation of treatment.

Further, the effectiveness of the 100 mg QD dosing regime in reducing blood pressure of hypertensive subjects is informative. In the 100 mg QD cohort of Example 2, the CYP 11β2 beta hydroxylase inhibitor was administered once daily in the morning. With an average time above IC50 of about 13.5 hours (see Example 1), the results of the 100 mg QD dosing regimen demonstrate that inhibition of CYP 11β2 beta hydroxylase for approximately half of the day is effective in reducing blood pressure in hypertensive subjects. In addition, the results from the 25 mg BID dosing regimen confirm that exposure to a CYP 11β2 beta hydroxylase inhibitor exceeding the IC50 for aldosterone production for approximately 12 hours is effective in reducing blood pressure regardless of whether this was achieved by once-daily or twice-daily dosing.

Thus, the studies described herein demonstrate that blood pressure can be effectively reduced in a hypertensive subject by once or twice daily administration of a CYP 11β2 beta hydroxylase inhibitor in an amount which inhibits 50% or more of the activity of CYP 11β2 beta hydroxylase for 40-60% of a 24 hour period.

### Example 3

### Methods

### In Vitro Studies

Assay of hCYP11B2 and hCYP11B1 Inhibition

The inhibitory effects of Compound A on human CYP 11B1 (hCYP11B1) and human CYP11B2 (hCYP11B2) enzyme activity were evaluated by determining the enzymatic conversion rates of 11-deoxycortisol to cortisol for hCYP11B1 and 11-DOC to aldosterone for hCYP11B2. Osilodrostat, which is a potent inhibitor of both hCYP11B1 and hCYP11B2, was used as a positive control. The inhibition constants of Compound A (free base) and osilodrostat on hCYP11B2 were calculated based on the rate of aldosterone generation (pg/μg protein/h) from the substrate 11-DOC with the mitochondrial fraction of V79 cells stably expressing hCYP11B2 as the enzyme source. Similarly, the inhibition constants of Compound A and osilodrostat on hCYP11B1 were calculated based on the rate of cortisol generation (pmol/mg protein/h) from the substrate 11-deoxycortisol with the mitochondrial fraction of V79 cells stably expressing hCYP11B1 as the enzyme source.

### Pan-Receptor Screening

Compound A, at a concentration of 10 μmol/L, was screened against 46 targets in receptor binding and enzyme inhibition studies to determine if Compound A interacted with any off-target enzymes or receptors.

Effects on hERG Current

The effects of Compound A on the human ether-à-go-go-related gene (hERG) current were evaluated using the whole-cell patch-clamp method in human embryonic kidney (HEK293) cells stably expressing the hERG channel. Cells were treated with Compound A at concentrations of 1, 3, 10, and 30 μmol/L, positive control (E-4031) at 0.1 μmol/L, or vehicle (0.3 v/v % dimethyl sulfoxide) for 10 minutes.

### Inhibitory Effects on Adrenal Steroidogenesis

Multiple concentrations of Compound A and aminoglutethimide (positive control) were evaluated in a human adrenocortical NCI-H295R cell culture. The effect of Compound A on adrenal steroidogenesis was determined by measurement of 12 steroid hormones in the culture medium after 3 days of incubation. The following steroids were measured: pregnenolone, 11-deoxycortisol, 17α-hydroxypregnenolone, 11-DOC, dehydroepiandrosterone, corticosterone, progesterone, cortisol, 17α-hydroxyprogesterone, aldosterone, androstenedione, and testosterone.

### In Vivo Animal Studies

All studies (except the ACTH-Treated Cynomolgus Monkey Model and Cardiovascular System in Halothane-Anesthetized Dogs studies) were performed at contract research organizations accredited by the Association for Assessment and Accreditation of Laboratory Animal Care (AAALAC) International. Definitive studies were conducted in compliance with Good Laboratory Practices (GLP) regulations at AAALAC-accredited contract research laboratories.

### Cardiovascular System in Halothane-Anesthetized Dogs

Effects of Compound A on the cardiovascular system were evaluated with halothane-anesthetized dogs. Doses of 0, 0.3, 1, and 3 mg/kg of Compound A as free base were infused intravenously in ascending order to the same 3 male dogs at 0.1 mL/kg/min for 10 minutes cumulatively with 30 minutes interval between the start of infusion. Heart rate, systolic, diastolic, and mean blood pressure, cardiac output, total peripheral resistance, left ventricular end-diastolic pressure, maximum upstroke velocity of the left ventricular pressure, and electrocardiogram parameters (PR interval, QRS duration, QT interval, and QTcV) were evaluated.

### Cardiovascular System in Telemetered Monkeys

Effects of Compound A on the cardiovascular system were evaluated with conscious telemetered cynomolgus monkeys. Single doses of 0, 10, 30, or 100 mg/kg of Compound A as free base were orally administered to four male monkeys in a Latin-square design with a 7-day interval between dosing. Blood pressure, heart rate, and electrocardiogram parameters (PR interval, QRS duration, QT interval, and QTcB) were evaluated up to 24 hours after each dosing.

### Sodium-Depleted Cynomolgus Monkey Model

A sodium-depleted cynomolgus monkey model with secondary hyperaldosteronism was performed to evaluate the effect of Compound A on plasma aldosterone concentrations (PACs). The model used a low-sodium diet and 3 doses of 5 mg/kg furosemide. Two animals each were assigned to 6 groups consisting of a vehicle group and 5 Compound A (free base) groups (0.3, 1, 3, 10, and 30 mg/kg). Six crossovers were performed so that every animal was assigned to each group. Hyperaldosteronism was induced once every 3 weeks. PAC was determined by radioimmunoassay and measured during the 24 hours after Compound A administration.

### ACTH-Treated Cynomolgus Monkey Model

An adrenocorticotropic hormone (ACTH)-treated cynomolgus monkey model was performed to evaluate the effect of Compound A on plasma cortisol concentrations (PCCs). A dose of Compound A, as free base, that was 100-fold higher than required to reduce PACs (0.3 mg/kg by mouth) was administered, followed immediately by ACTH (50 μg/kg subcutaneously). PACs were also evaluated in the animals. PACs and PCCs were determined by radioimmunoassay and measured during the 24 hours after Compound A administration.

### Toxicology Studies

The toxicologic profile of Compound A was assessed in several toxicology studies, including 13-week repeat-dose oral studies in Sprague Dawley rats and cynomolgus monkeys, a standard battery of genotoxicity studies, preliminary embryo-fetal development studies in rats and rabbits, and in vitro and in vivo phototoxicity studies. Rats and monkeys were selected for the general toxicology studies on the basis of pharmacology and metabolism data that indicated these animals would have metabolism similar to that of humans. Studies were performed in accordance with GLP regulations.

Doses in the 13-week toxicology studies were 0, 50, 150, 450 (female), and 600 (male) mg/kg/d in Sprague Dawley rats. In cynomolgus monkeys (females and males), the doses were 0, 10, 30, and 100 mg/kg/d. Hematoxylin-and-eosin-stained sections (nominal thickness of approximately 5 μm) were prepared from the formalin-fixed paraffin-embedded organs/tissues of all animals in the toxicology studies.

### Clinical Studies

A 4-part, Phase 1 randomized, double-blind, placebo-controlled, first-in-human study was performed to determine the safety, tolerability, PK, and PD of single ascending doses (SADs; part 1) and multiple ascending doses (MADs; part 2) of Compound A HBr in healthy participants. The study also assessed whether there were sex (part 3) and age-related (part 4) effects on the PK of a single dose of Compound A HBr. The study was approved by the local independent ethics committee; the BEBO foundation in Assen, Netherlands; and the Central Committee on Research Involving Human Subjects in The Hague, Netherlands; and conducted according to the provisions of the Declaration of Helsinki. Written informed consent was obtained from each study participant before conducting any protocol-related procedures.

In part 1, Compound A HBr or placebo was administered to 64 healthy Caucasian men aged 18 to 55 years (inclusive) in 8 cohorts of 8 participants each. In each cohort, 6 participants were randomized to receive a single dose of Compound A HBr and 2 participants were randomized to receive matching placebo. All cohorts included 2 sentinel participants of whom 1 received Compound A HBr and 1 received matching placebo. The remaining 6 participants, of whom 5 received Compound A HBr and 1 received placebo, were dosed ≥24 hours following the sentinel participants. The dosing cohorts were 5, 10, 20, 50, 100, 200, 400, and 800 mg. Doses were administered in an ascending order per cohort, with a minimum 10-day interval between consecutive dose levels. Participants received a single dose of Compound A HBr or placebo in the fasted state.

In part 2, Compound A HBr or placebo was administered to a total of 36 healthy Caucasian men aged 19 to 54 years (inclusive) in 3 cohorts of 12 participants each. In each cohort, 9 participants were randomized to receive multiple doses of Compound A HBr and 3 participants were randomized to receive matching placebo. The participants received daily doses in the fed state from Day 1 to Day 7. The doses were administered in an ascending order per cohort. Progression to the next dose level and dose selection were based on available data from part 1 and the preceding dose cohort in part 2 (safety, tolerability, and PD data [up to 48 hours post-final dose] and available PK data [up to 24 hours post-final dose] from a minimum of 10 participants [Compound A HBr n≥7] in the preceding dose cohort). The final dose levels of Compound A HBr for part 2 were 40, 120, and 360 mg. An ACTH challenge test was performed on Day −2 and on Day 6 to evaluate Compound A HBr selectivity for aldosterone synthesis. Plasma concentrations of aldosterone and 11-deoxycortisol and serum concentrations of cortisol and 11-DOC were measured pre-ACTH dose and 30 and 60 minutes post-ACTH dose on Day −2 and Day 6.

Part 3 of the study evaluated the sex-related effects on the safety, tolerability, and PK of Compound A HBr. In 1 cohort of 8 healthy Caucasian women aged 20 to 35 years (inclusive), 6 participants were randomized to receive a single 100-mg dose of Compound A HBr and 2 participants were randomized to receive matching placebo. Compound A HBr or placebo was administered in the fasted state. The results were compared with those in the cohort at the same dose as in Part 1.

Part 4 of the study evaluated the age-related effects on safety, tolerability, and PK of Compound A HBr. In 1 cohort of 8 healthy Caucasian men aged 68 to 80 years, 6 participants were randomized to receive a single 100-mg dose of Compound A HBr and 2 participants were randomized to receive matching placebo. Compound A HBr or placebo were administered in the fasted state. The results were compared with those in the cohort at the same dose as in Part 1.

### Statistical, Pharmacokinetic, and Pharmacodynamic Analysis

Safety measurements (adverse events [AE], safety laboratory, vital signs and electrocardiograms, physical examinations) were summarized and/or listed.

### Pharmacokinetics

Plasma PK parameters were derived by non-compartmental analysis using WinNonlin® (version 6.3). Dose proportionality was assessed using the power model with AUC_{0-∞}, AUC₀₋ₗₐₛₜ, and Cₘₐₓ for part 1 (SAD) and with AUC₀₋ₗₐₛₜ, AUC_{0-∞}, AUC_{0-τ}, and Cₘₐₓ for part 2 (MAD). A linear model (ln(Y)=α+β×ln(X), where Y is the pharmacokinetic parameter and X is the dose) was used to fit the power model, after log-transformation of the parameters. Dose proportionality was concluded if the 95% confidence interval (CI) for slope (β) included the value 1.

AUC₀₋ₗₐₛₜ, AUC_{0-∞}, and Cₘₐₓ were used to explore any sex and age-related effects (part 3 and 4, respectively). A linear model was used to analyze log-transformed AUC and Cₘₐₓ with sex or age as fixed effects. Difference in least square means (LSM) and corresponding 90% CI were back-transformed to obtain the estimates of geometric mean ratios and their CI for females vs. males and ≥65 years vs. <65 years participants. The result was judged to be statistically significant if the 90% CI did not include 1.

### Pharmacodynamics

Plasma concentrations of aldosterone, 11-deoxycortisol, and ACTH; renin activity; renin concentration; and serum concentrations of cortisol and 11-DOC were measured in all parts of the study. The amount of aldosterone, cortisol, sodium, and potassium excreted and the urinary log₁₀ (10×Na⁺/K⁺) ratio were listed per collection interval.

PD parameters were derived by non-compartmental analysis using WinNonlin® Professional (version 6.3). AUC₀₋₂₄ was determined where possible for plasma aldosterone and serum cortisol.

Log-transformed change from baseline (Day 1, pre-dose) at 24 hours and log-transformed change from baseline at 22 hours on Day 1 (where baseline is Day −1, 22 hours) in plasma aldosterone and serum cortisol were analyzed using a linear model with dose group (each Compound A HBr dose and pooled placebo) as a fixed effect with the corresponding Day 1 pre-dose as a covariate. The LSM difference between each Compound A HBr dose compared with placebo was obtained from the model with the 90% CI and then back-transformed (exponentiated) to obtain estimates of the ratio of adjusted geometric means and 90% CI. Log-transformed change from baseline (time-matched) from 0 to 24 hours post-dose in plasma aldosterone and serum cortisol as repeated measures was analyzed using a linear mixed model with dose group (each active Compound A HBr dose and pooled placebo) as fixed effects, corresponding time-matched values on Day −1 as covariate, and unstructured covariance. The pre-dose time point on Day 1 was used as the 24-hour time point on Day −1. The LSM difference between each Compound A HBr dose compared with placebo was obtained from the model together with the 90% CI, and then back-transformed to obtain estimates of the ratio of adjusted geometric means and 90% CIs. The calculated AUC parameters were analyzed using a linear model. The log-transformed parameter of interest was the dependent variable with dose group (each Compound A HBr dose and pooled placebo) as the fixed effect and log-transformed AUC₀₋₂₄ on Day −1 as the covariate. The LSM difference between each Compound A HBr dose compared with placebo and 90% CIs was calculated and then back-transformed to obtain estimates of the ratio of adjusted geometric means and 90% CIs.

### Analytical Methods

An analytical method for the determination of Compound A in human plasma (dipotassium ethylenediaminetetraacetic acid [K2-EDTA]) and human urine using solid-phase extraction and high-performance liquid chromatography with mass spectrometric detection was successfully validated over the concentration range 0.1000 ng/mL (lower limit of quantification) to 100.0 ng/mL prior to initiation of the clinical trial. A quantitative analytical method for the determination of 11-DOC in human ethylenediaminetetraacetic acid (EDTA) plasma samples using ultra-high-performance liquid chromatography with tandem mass spectrometric detection was successfully validated over the concentration range of 0.0400 ng/mL (lower limit of quantification) to 32.2 ng/mL. For all other analytes, validated quantitative measurement methods were used.

### Results

In Vitro hCYP11B2 and hCYP11B1 Inhibition

Assessment of the pharmacologic profile for Compound A demonstrated that Compound A inhibited hCYP11B2 and hCYP11B1 with inhibition constant values of 1.27 nmol/L and 475 nmol/L, respectively. In comparison, the reference compound osilodrostat inhibited hCYP11B2 and hCYP11B1 with inhibition constant values of 0.151 nmol/L and 0.546 nmol/L, respectively. Compound A showed a much higher selectivity for hCYP11B2 compared with osilodrostat (Table 2). These results indicate that Compound A inhibits CYP11B2 with 374-fold higher selectivity over CYP11B1.

TABLE 2
hCYP11B2 and hCYP11B1 inhibition.

Inhibition constant values (Ki)
Test Drug

| CYP450 | Compound A | Osilodrostat |
|---|---|---|
| hCYP11B2 | 1.27 nmol/L^{a} | 0.151 nmol/L |
| hCYP11B1 | 475 nmol/L | 0.546 nmol/L |

hCYP, human cytochrome P450.
^{a}Free base.

### Preclinical Pharmacology Screens

Compound A at a concentration of 10 μmol/L did not substantially inhibit activity of any of the 46 primary molecular target receptors or enzymes. Half-maximal inhibitory concentration (IC50) of Compound A for any of these off-target receptors or enzymes is >10 μmol/L, which indicated a low risk for off-target pharmacologic effects.

Compound A inhibited hERG current by 27% at 30 μmol/L, indicating that the hERG IC50 is >30 μmol/L. In halothane-anesthetized dogs, no effects were noted on the cardiovascular system following intravenous infusion up to 3-mg/kg Compound A as free base under non-GLP conditions. In telemetered monkeys, there were no effects on the cardiovascular parameters including blood pressure, heart rate, or electrocardiogram following single oral doses up to 100-mg/kg Compound A as free base. In the 13-week toxicity study in monkeys, there were no Compound A-related effects on electrocardiogram values at doses up to 100 mg/kg/d. These studies indicate a low level of risk for adverse cardiovascular effects with Compound A, especially cardiac electrophysiology.

### Inhibitory Effects on Adrenal Steroidogenesis

In the human adrenocortical NCI-H295R cell culture, the effect of Compound A on adrenal steroidogenesis was determined by measurement of 12 steroid hormones after 3 days of incubation. The effects on adrenal steroidogenesis were most profound on aldosterone production. Compound A concentrations ≥0.11 μmol/L resulted in >50% reduction in aldosterone secretion; similar reductions (>50%) were also observed in corticosterone at concentrations ≥1 μmol/L. The reductions in pregnenolone, progesterone, 17α-hydroxyprogesterone, 11-DOC, corticosterone, and cortisol at the top concentration (3 μmol/L) did not result in a reduction of ≥50%. All effects on adrenal steroidogenesis were mechanism-based, and nonspecific inhibitory effects were not observed.

### Adrenal Histopathology in the 13-Week Toxicology Studies

In the 13-week, long-term, daily dosing toxicology study in rats, the histopathological findings at doses ≥150 mg/kg/d included vacuolation of zona fasciculata cells and hypertrophy of zona glomerulosa cells in the adrenal glands, consistent with the anticipated pharmacologic effects of Compound A. The no observed adverse effect level (NOAEL) was 50 mg/kg/d.

In the 13-week long-term daily dosing toxicology study in monkeys, dose-related effects upon aldosterone precursors and aldosterone were noted with no effects on cortisol. Hypertrophy of zona glomerulosa cells in the adrenal cortices and decreased PACs were observed at all dose levels. Adrenal cortical glomerular hypertrophy was noted across all Compound A-treated groups and was minimally noted in one animal at 100 mg/kg/d after a 4-week recovery period. Minimal cell death in the zona glomerulosa was observed in Compound A-treated animals (100 mg/kg/d) and in one female at 30 mg/kg/d with no observations following a 4-week recovery period. The NOAELs were 30 mg/kg/d for male animals and 10 mg/kg/d for female animals.

### Pharmacologic Effects in Animal Studies

Single oral administration of Compound A significantly decreased PAC in a sodium-depleted monkey model. However, single oral administration of Compound A did not affect PCCs in ACTH-loaded monkeys at a dose 100 times greater than the dose that inhibited PAC production. These results indicate that Compound A inhibits CYP11B2 with 100-fold higher selectivity over CYP11B1.

### Clinical Studies

A total of 245 participants were screened, of which 116 were randomized. All participants randomized in parts 1, 3, and 4 completed the study as per protocol. One participant in cohort 3 of part 2 was withdrawn on Day 2 because of an AE of sinus tachycardia and received only one dose of 360 mg Compound A HBr on Day 1.

### Safety and Tolerability

No serious AEs occurred during the study. The overall incidence of TEAEs was comparable between participants treated with Compound A HBr (41 of 87 [47%]) and placebo-treated participants (18 of 29 [62%]). Across all cohorts, dizziness (of mild intensity) was reported by 9 (10.3%) Compound A HBr-treated participants compared with 1 (3.4%) placebo participant. No other trends were identified in the frequency of TEAEs across single or multiple dose levels of Compound A HBr.

### Pharmacokinetics

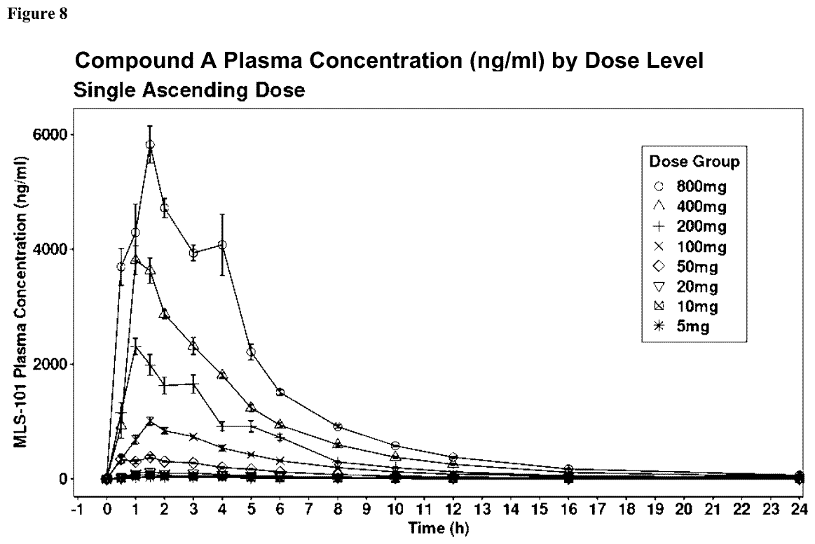

Over the dose range of 5 to 800 mg of Compound A HBr, the 95% CIs of the slopes for the AUCs included unity, indicating a dose-proportional increase in the systemic exposure in terms of AUC₀₋ₗₐₛₜ and AUC_{0-∞}. In contrast, the estimate of the slope (95% CI) for Cₘₐₓ was 1.104 (1.043-1.164) and a slightly greater than dose-proportional increase in Cₘₐₓ was observed over the range 5 to 800 mg (FIG. 8; Table 3). Median Tₘₐₓ values ranged between 1 and 1.5 hours and mean t_{1/2} values ranged from 7.92 and 10.54 hours.

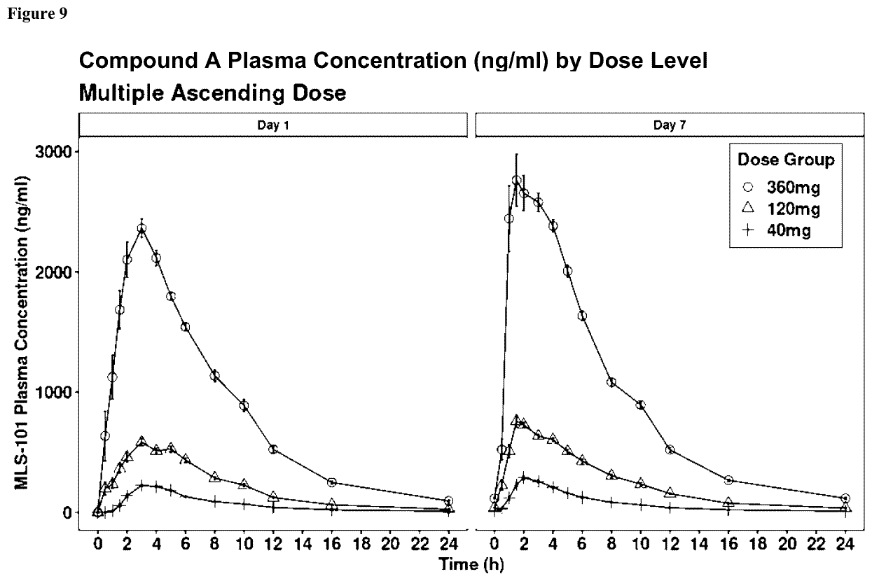

After 7 days of once-daily administration of oral Compound A HBr doses ranging from 40 to 360 mg, systemic exposure in terms of AUC and Cₘₐₓ appeared to increase in a slightly more than dose-proportional manner over the tested multiple dose range on Days 1 and 7 (FIG. 9; Table 4). For AUC and Cₘₐₓ on Day 1, the estimates of the slopes ranged from 1.136 to 1.146 (95% CI, 1.011-1.281), and for AUC0-τ on Day 7, the estimate of the slope was 1.147 (95% CI, 1.060-1.234). Steady state was achieved by approximately Day 5 and very slight accumulation of Compound A HBr was observed following multiple dosing compared with Day 1, with mean rate of accumulation values ranging from 1.15 to 1.19 across the entire dose range. Consistent with single-dose administration, the t_{1/2} on Day 7 was approximately 10 hours. After a single 100-mg dose of Compound A HBr, the exposure in terms of Cₘₐₓ and AUC was 25% and 19% higher in female participants than in male participants, respectively, but the differences were not statistically significant based on 90% CIs. The Cₘₐₓ and AUC were approximately 14% and 12% lower in men aged ≥65 years vs those aged <65 years, respectively. Sex- and age-related effects on the PK of Compound A HBr were not statistically significant based on 90% CIs.

TABLE 3
Pharmacokinetics for single ascending dose groups, mean (SD).

| | | AUC_{0-inf}, | Cₘₐₓ, | | |
|---|---|---|---|---|---|
| Dose Group | | ng × h/mL | ng/mL | T 1/2, h | Tₘₐₓ, h |
| 5 | mg | 231 (25) | 37 (7) | 8 (1) | 1.5 (1-3) |
| 10 | mg | 451 (46) | 76 (20) | 8 (1) | 1.25 (1-1.5) |
| 20 | mg | 768 (182) | 141 (76) | 10 (2) | 1.5 (1-3) |
| 50 | mg | 2155 (258) | 573 (206) | 9 (2) | 1 (0.5-3) |
| 100 | mg | 5366 (493) | 1211 (248) | 10 (1) | 1.5 (1-3) |
| 200 | mg | 10645 (3486) | 2848 (1015) | 10 (4) | 1.25 (1-3) |
| 400 | mg | 17257 (2907) | 4455 (862) | 10 (2) | 1.25 (0.5-3) |
| 800 | mg | 29331 (3623) | 7708 (1617) | 11 (2) | 1.5 (0.5-4) |

AUC_{0-inf}, area under the curve from 0 to inf; Cₘₐₓ, maximum concentration; Tₘₐₓ, time to reach the maximum concentration, median and range.

TABLE 4
Pharmacokinetics for multiple ascending dose groups, mean (SD).

| | | | AUC₀₋₂₄, | Cₘₐₓ, | | |
|---|---|---|---|---|---|---|
| Dose Group | | Day | ng × h/mL | ng/mL | T 1/2, h | Tₘₐₓ, h |
| 40 | mg | 1 | 1574 (282) | 252 (64) | 5 (1) | 3 (0.5-5) |
| 40 | mg | 7 | 1795 (312) | 365 (46) | 9 (2) | 1.5 (0.5-5) |
| 120 | mg | 1 | 4876 (1024) | 887 (291) | 4 (1) | 3 (0.5-8) |
| 120 | mg | 7 | 5816 (1315) | 1039 (343) | 12 (2) | 2.5 (1-4) |
| 360 | mg | 1 | 19335 (3111) | 3220 (1171) | 4 (1) | 3 (2-5) |
| 360 | mg | 7 | 21825 (3955) | 3812 (1282) | 9 (2) | 2 (1-3) |

AUC₀₋ₜ, area under the curve from 0 to 24 Cₘₐₓ, maximum concentration; Tₘₐₓ, time to reach the maximum concentration, median and range.

### Pharmacodynamics

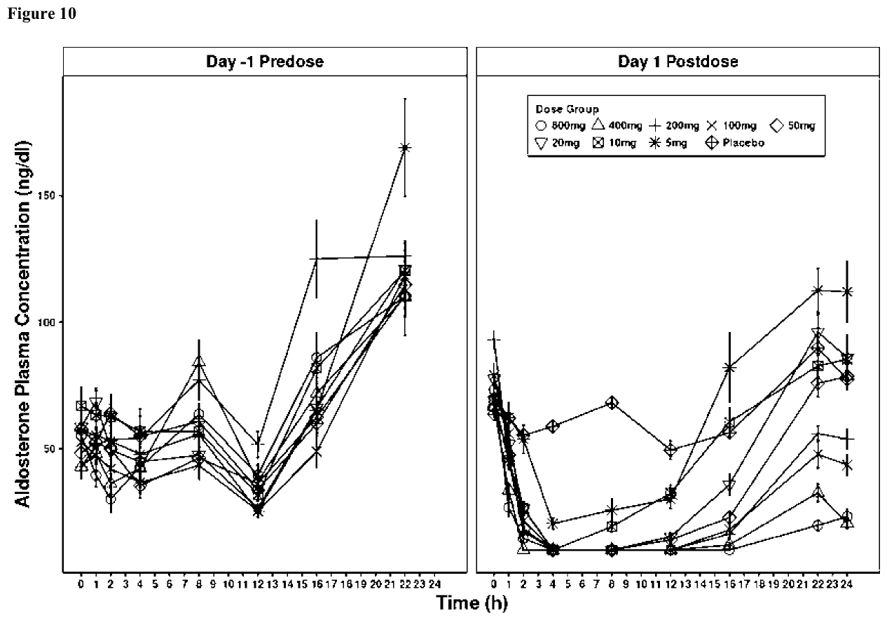

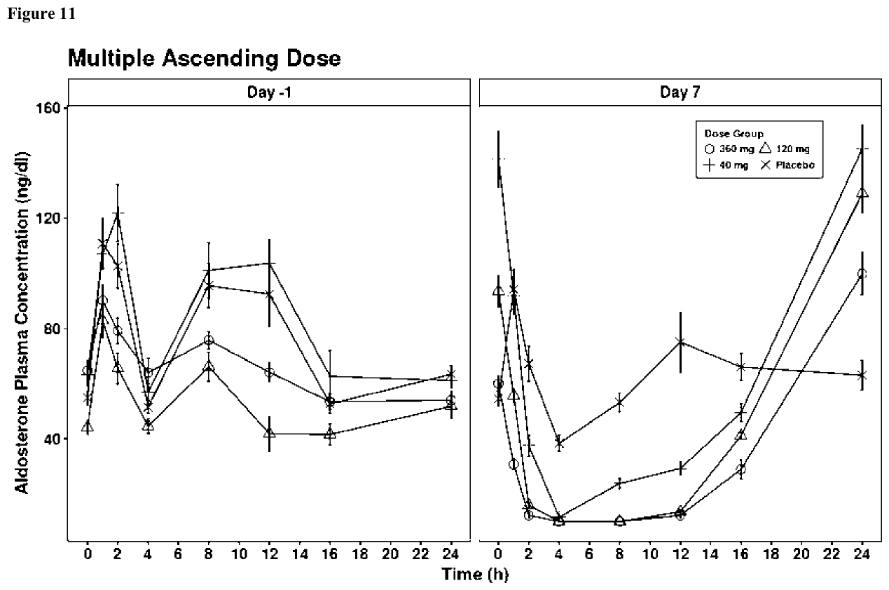

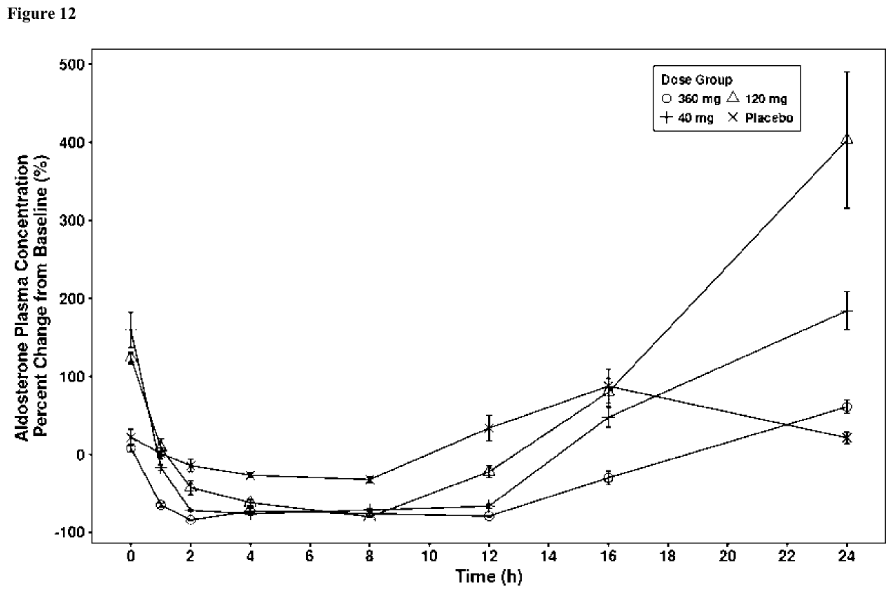

All single dose levels of Compound A HBr showed a clear reduction in PAC compared with baseline at 4 hours and 8 hours post-dose (FIG. 10). At the lower dose levels (Compound A HBr 5-50 mg), the PAC returned to values near baseline within 24 hours post-dose. At the higher dose levels (Compound A HBr 100-800 mg), the reduction of PAC was sustained until 24 hours post-dose. At 24 hours post-dose, single doses of Compound A HBr reduced PAC up to approximately −40% at doses of 100 and 200 mg and up to approximately −70% at doses of 400 and 800 mg compared with placebo. Decreases in PAC were statistically significant at doses of 100- to 800-mg Compound A HBr). PAC showed maximum decreases at 4 hours and 8 hours post-dose (geometric mean ratio [90% CI]: 0.20 [0.15-0.26] and 0.15 [0.11-0.21], respectively, for 400-mg dose over pooled placebo). Single doses of Compound A HBr, ranging from 10 to 800 mg, particularly reduced the AUC₀₋₂₄ for PAC in a dose-dependent manner, with statistically significant decreases ranging from −36% to −77% vs placebo (Table 5 and Table 6). Single 100-mg doses of Compound A HBr and multiple 120-mg doses of Compound A HBr allowed for the return of baseline aldosterone levels by 16 hours (FIG. 10 to FIG. 12).

In parts 3 and 4, the change from baseline of PAC and serum cortisol concentrations at 24 hours after a single 100-mg dose of Compound A HBr was similar in male and female participants, as well as in those aged <65 years and aged ≥65 years.

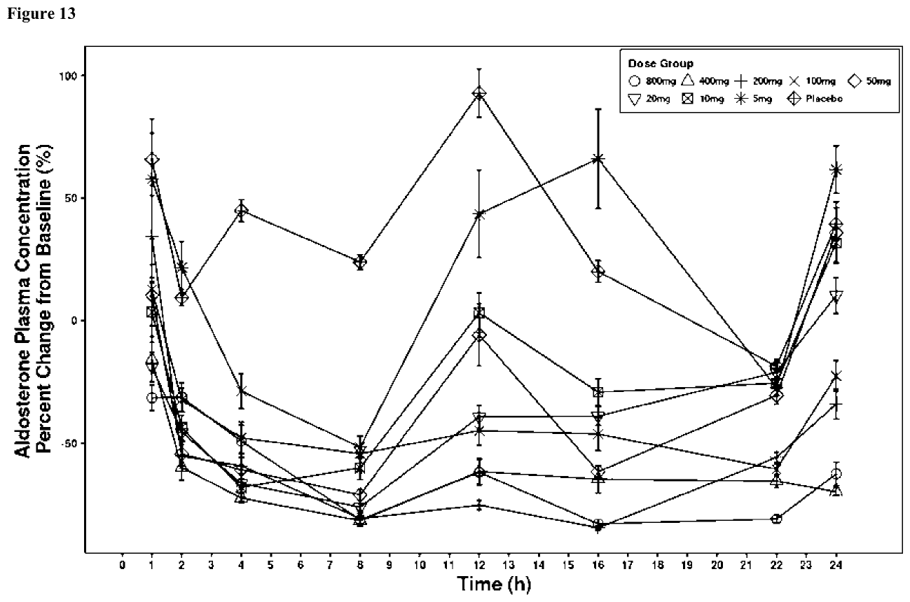

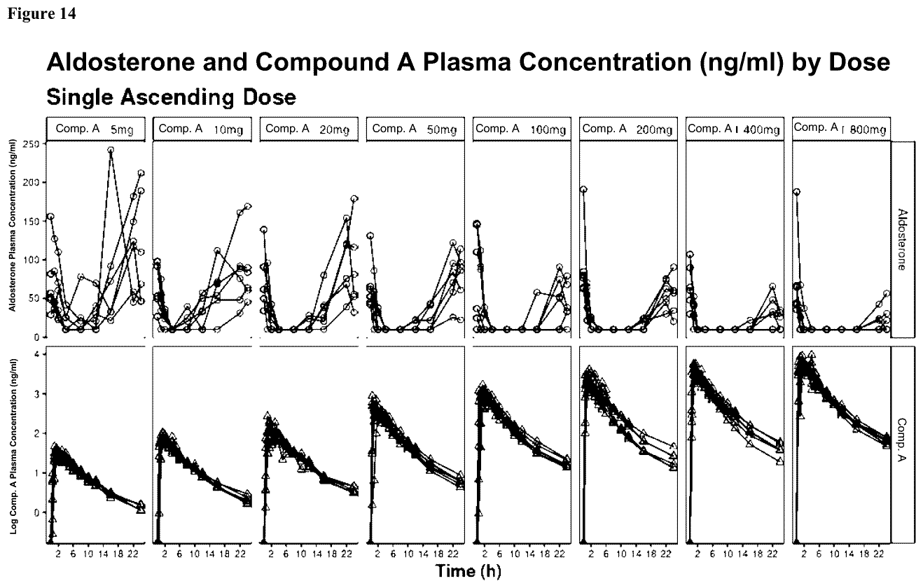

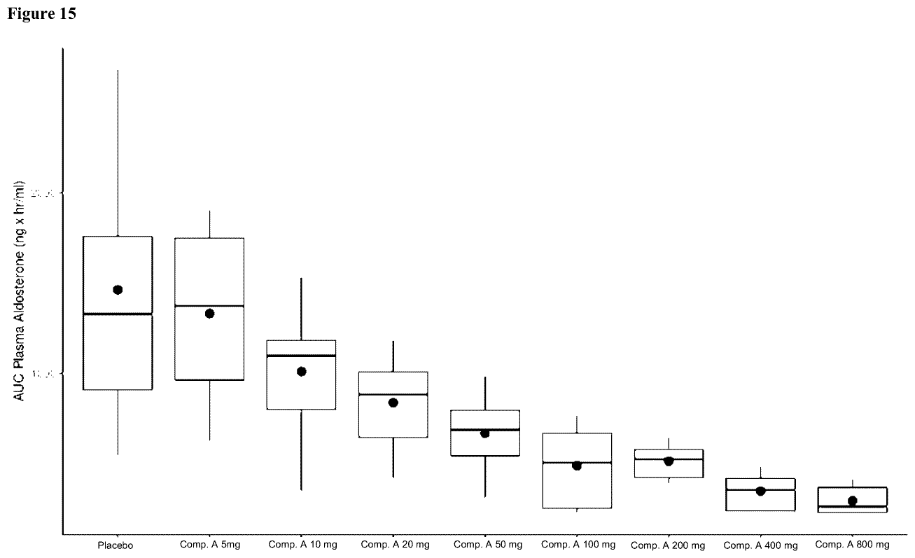

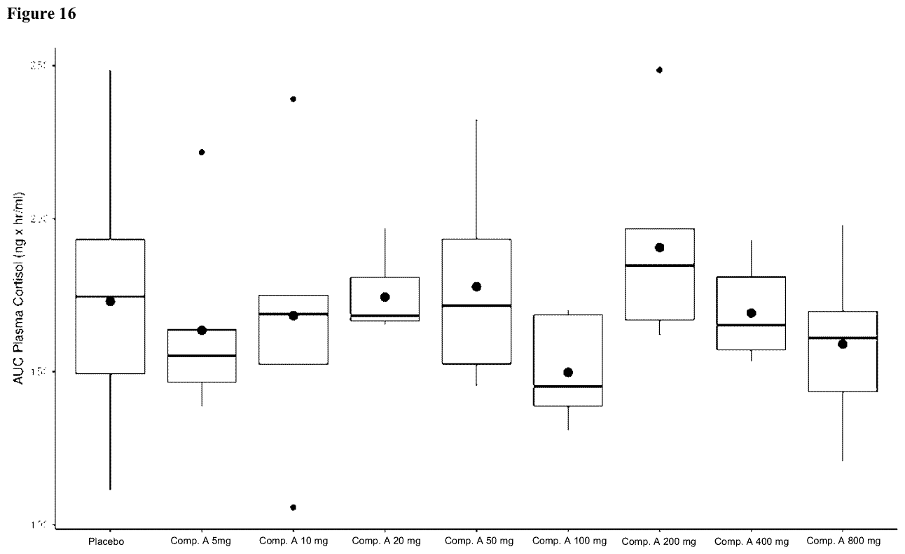

The time profile for percent change from baseline for aldosterone for SADs demonstrated the return to baseline for doses of 20 mg or lower (FIG. 13). The relationship between individual PK and the time course of aldosterone suppression and recovery in the SAD groups demonstrated a clear PK-PD relationship (FIG. 14). The suppressive effect of Compound A HBr on aldosterone was dose related and demonstrated no dose-related effect on cortisol in SAD (FIG. 15 and FIG. 16).

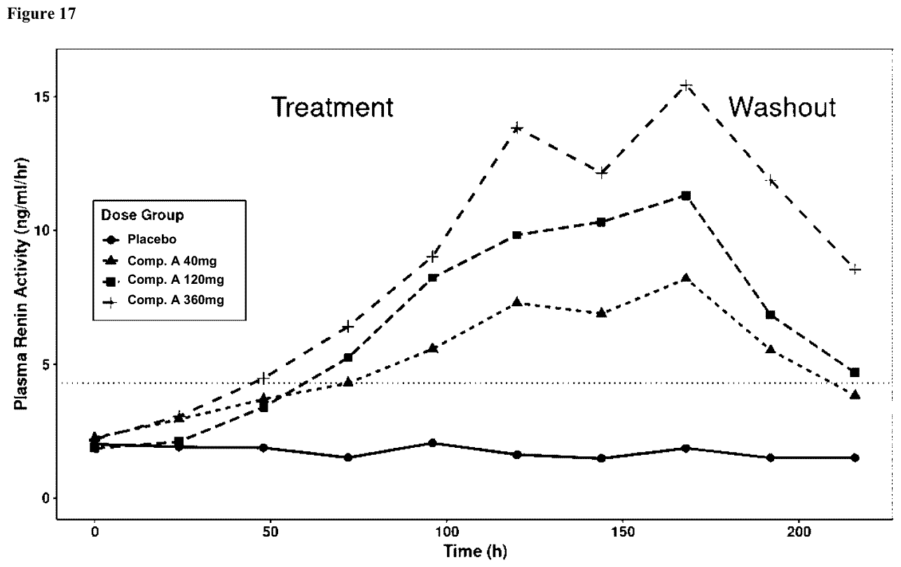

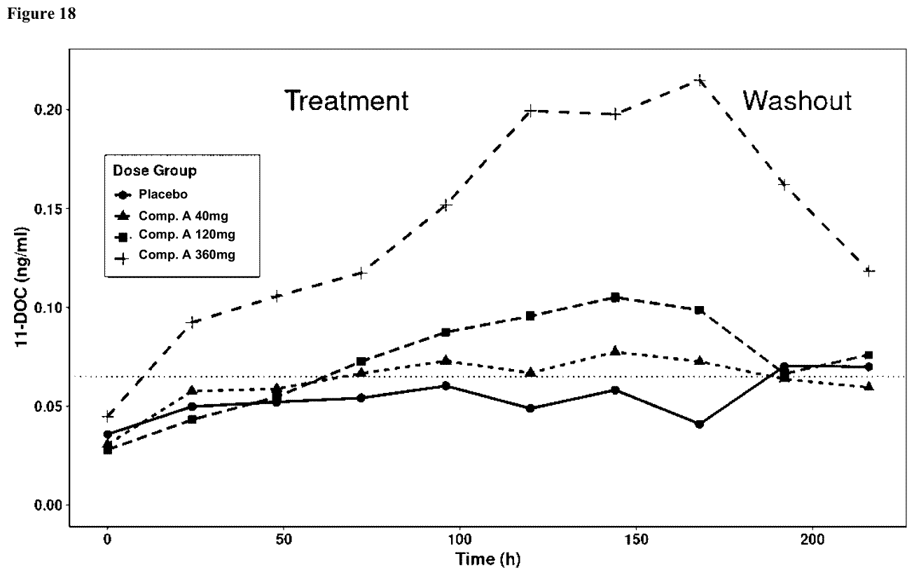

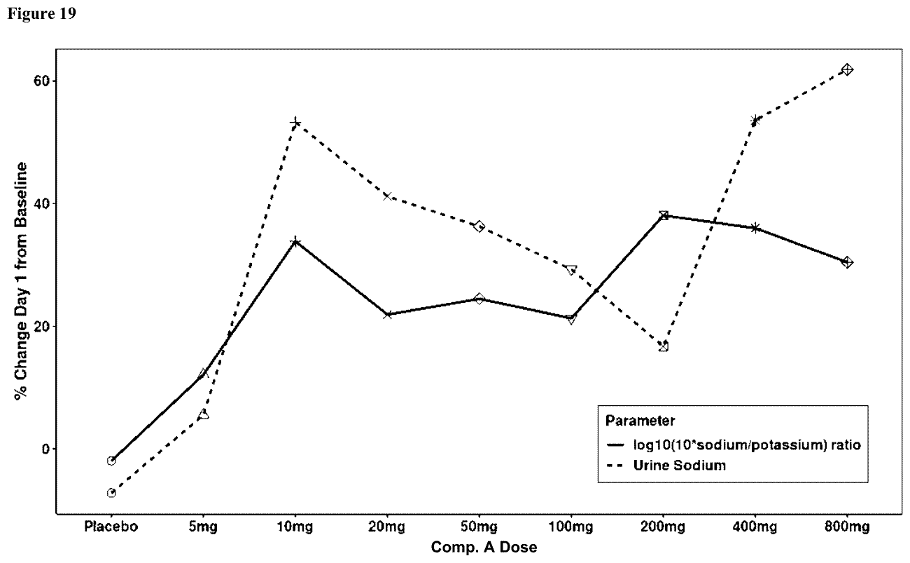

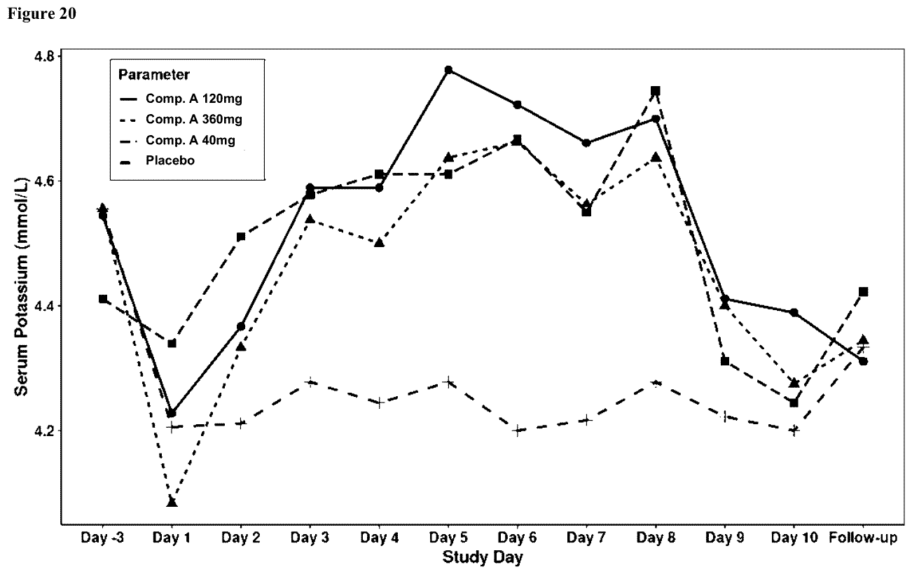

The effect of Compound A HBr on increased plasma renin activity and 11-DOC from the MAD and the effects of Compound A HBr on renal sodium and potassium handling from the SAD and MAD portions of the study indicate increases in urine sodium and urine Na+/K+ ratios as well as a modest effect on increased plasma K+ (FIG. 17, FIG. 18, FIG. 19, and FIG. 20). The aldosterone AUCs are shown in Table 5 and Table 6.

Within 24 hours after seven once-daily doses, a statistically significant reduction based on 90% CI in the AUC0-24 for PAC of approximately −50% compared to placebo was observed at a dose of 360 mg Compound A HBr. For all dose levels tested, PAC decreased in an apparent dose-dependent manner between 2 and 12 hours post-dose on Day 7. Particularly at the lower multiple dose levels (40 and 120 mg) the PAC reductions were less pronounced than observed after single doses of Compound A HBr. Compared to placebo, PAC was reduced at 24 hours on Days 2 to 5 for the 360 mg dose only, with no change observed on Days 6 and 7. From 24 hours after the last dose on Day 7, a clear rebound in PAC ranging from 70% to 160% compared to placebo was observed at all multiple dose levels of Compound A HBr. These increases were sustained at least until Day 10, 3 days after the last dose. No relevant changes from baseline were observed for serum cortisol concentrations and plasma 11-deoxycortisol concentrations during multiple dosing of Compound A HBr.

At all multiple dose levels, Compound A HBr completely blunted the ACTH-stimulated aldosterone response on Day 6 compared to placebo while there was no effect on the cortisol response. At 360 mg of Compound A HBr, ACTH stimulation on Day 6 showed a trend in increase in 11-DOC and 11-deoxycortisol concentrations compared to placebo. These results confirm that Compound A is a potent and highly selective inhibitor of CYP11B2 that decreases PACs without affecting serum cortisol concentrations in healthy volunteers.

Furthermore, once-daily administration of Compound A appeared to increase serum 11-DOC, plasma renin activity, and potassium concentrations, at all dose levels compared to placebo. These effects wore off as soon as dosing was stopped. Also, the urinary log 10 (10×Na⁺/K⁺) ratio was initially increased on Day 1 at all multiple dose levels, but the ratio returned to baseline levels at the next sampling time point at the end of the dosing period on Day 7. From Day 7 to Day 9, clear reductions in the urinary log 10 (10×Na⁺/K⁺) ratio were observed suggesting a rebound-effect for the sodium/potassium ratio, as well.

TABLE 5
Aldosterone AUC₀₋₂₄ for the single ascending
dose groups, Day 1, mean (SD).

| Dose Group | AUC₀₋₂₄, ng × h/dL |
|---|---|
| Placebo | 1543 (657) |

| | | |
|---|---|---|
| 5 | mg | 1436 (531) |
| 10 | mg | 1075 (418) |
| 20 | mg | 911 (286) |
| 50 | mg | 731 (247) |
| 100 | mg | 562 (279) |
| 200 | mg | 587 (115) |
| 400 | mg | 403 (122) |
| 800 | mg | 347 (108) |

AUC₀₋₂₄, area under the curve from 0 to 24 hours

TABLE 6
Aldosterone AUC₀₋₂₄ for the multiple ascending
dose groups, Day 7, mean (SD).

| Dose Group | Day | AUC₀₋₂₄, ng × h/dL |
|---|---|---|
| Placebo | 7 | 1499 (573) |
| 40 mg | 7 | 1343 (702) |
| 120 mg | 7 | 1013 (312) |
| 360 mg | 7 | 771 (440) |

AUC₀₋₂₄, area under the curve from 0 to 24 hours

### Discussion

The renin-angiotensin-aldosterone system (RAAS) plays a central role in the control of intravascular volume, blood pressure, and serum potassium concentration. Yin, L. et al. (2012); Nehme, A. et al. (2019). Normally, short and long negative feedback loops maintain homeostasis in the RAAS system. Chong, C. et al. (2017) Low intravascular volume increases renal pro-renin expression, leading to increased renin-mediated conversion of angiotensinogen to angiotensin-1 (Ang-1), angiotensin-converting enzyme (ACE) conversion of Ang-1 to angiotensin-2 (Ang-2), and Ang-2 binding to the type 1 angiotensin receptor in the adrenal cortex, stimulating production of aldosterone. Laragh, J. H. & Sealey J. E. (2011); Nehme, A. et al. (2019). Increased aldosterone then binds to the mineralocorticoid receptor (MR), ultimately increasing sodium reabsorption in the distal nephron. Yin, L. et al. (2012); Atlas, S. A. (2007); Brown, J. M. et al. (2020). This sodium reabsorption increases intravascular volume, thus reducing pro-renin production, closing the long negative feedback loop, and ensuring homeostasis. Yin, L. et al. (2012). A short feedback loop in the adrenal gland plays a complementary role, such that increased aldosterone in the adrenal gland binds to the MR locally and moderates further production of aldosterone. Chong, C. et al. (2017).

Because of the importance of the RAAS system in maintaining control of volume and blood pressure, each step in the pathway has been explored as a therapeutic target for the treatment of hypertension. The earliest therapeutic agent used in humans was spironolactone, a steroidal inhibitor of the MR, but its use has been hampered by adverse (notably estrogenic) effects. Brown, J. M. et al. (2020); Williams, B. et al. (2015). More recently, improved non-steroidal MR inhibitors have also been developed. Multiple ACE inhibitors and angiotensin receptor blockers (ARBs) have been in use since the late 20th century. Laragh, J. H. & Sealey J. E. (2011); Atlas, S. A. (2007). Most recently, renin inhibitors have been added to the therapeutic armamentarium, providing broad targeting of the upstream components of the pathway. Laragh, J. H. & Sealey J. E. (2011); Atlas, S. A. (2007). Unfortunately, each of these approaches has significant limitations, and a high unmet medical need remains. Atlas, S. A. (2007).

One such limitation is the hyperkalemia associated with MR antagonists, especially in chronic kidney disease (CKD). Additionally, cortisol binds to the MR, and the MR is co-localized with an enzyme that degrades cortisol. In CKD, this activity is low and cortisol stimulates the MR. Blocking the effect of both aldosterone and cortisol in CKD can result in hyperkalemia. Pfizer (2020). Since aldosterone synthase inhibitors (ASIs) do not interfere with the binding of corticosteroids to the MR, they can function as weak agonists on the MR, resulting in less hyperkalemia in patients with impaired renal function. Deleterious organ remodelling, such as vascular smooth muscle cell hypertrophy, cardiovascular fibrosis, and interstitial fibrosis of the kidney, is mediated by aldosterone at least partly via non-MR dependent effects of aldosterone.

Prevention of angiotensin II generation or action would be expected to prevent the secretion of aldosterone, but this is not universally the case. A trial in left ventricular dysfunction (RESOLVD) showed that RAAS suppression with an ACE inhibitor and an ARB did not suppress long-term aldosterone secretion. This phenomenon has been described as ‘aldosterone escape.’ Complicating the situation further is the increasing recognition of classical hyperaldosteronism and obesity-associated inappropriate aldosterone production as underlying causes of treatment-resistant hypertension. Brown, J. M. et al. (2020); Calhoun, D. A. et al. (2002); Calhoun, D. A. (2016). Overall, the global prevalence of treatment-resistant hypertension is estimated to be 10.3%, but the rate is higher among patients with CKD, renal transplant recipients, and the elderly (22.9%, 56.0%, and 12.3%, respectively). Noubiap J. J. et al. (2018). Salt-sensitive hypertension is commonly associated with both obesity and African American ancestry, though the mechanisms of theses associations are not firmly established. Calhoun, D. A. et al. (2002); Calhoun, D. A. (2016). These diverse individuals are generally not considered to be equivalent with the classic hyperaldosterone population and are perhaps better thought of as having an acquired abnormality in systems biology, interfering with the normal feedback loops that control aldosterone production.

Primary aldosteronism (PA) is a syndrome of non-suppressible, renin-independent aldosterone production that causes hypertension and cardiovascular disease. It may occur in >20% of patients with resistant hypertension and is the most prevalent cause of secondary hypertension. Brown, J. M. et al. (2020); Calhoun, D. A. (2016); Parasiliti-Caprino, M. et al. (2020); Pfizer (2020); Strauch, B. et al. (2003). PA is usually caused by an adrenal adenoma or unilateral or bilateral adrenal hyperplasia (BAH); in rare cases, it may be caused by an adrenal carcinoma or inherited conditions of familial hyperaldosteronism. Unilateral adenoma can be cured surgically, while MR antagonists are the treatment of choice for non-surgically resectable causes. Compared with primary (or essential) hypertension, PA causes more end-organ damage and is associated with excess cardiovascular morbidity, including heart failure, stroke, nonfatal myocardial infarction, and atrial fibrillation. Byrd, J. B. (2015); Monticone, S. et al. (2017). Biochemically, PA is defined by high aldosterone-renin ratios and confirmed by a fludrocortisone suppression test, captopril challenge test, or saline loading test. Calhoun, D. A. et al. (2002); Strauch, B. et al. (2003).

Evidence in support of aldosterone inhibition as a viable approach to the treatment of resistant hypertension was provided by the PATHWAY-2 trial. Williams, B. et al. (2015). Individuals with the lowest plasma renin activity, indicative of aldosterone-mediated negative feedback on renin production, showed a dramatic reduction in blood pressure when treated with spironolactone. This contrasted with the relatively modest blood pressure reduction seen in individuals with normal-to-high plasma renin and was not achieved with α- or β-blockers. Williams, B. et al. (2015). In that relatively small trial, 25 to 50 mg of spironolactone once daily demonstrated an acceptable safety profile. However, MR blockers have been associated with an approximately 10% incidence of clinically meaningful, and occasionally life-threatening, hyperkalemia—particularly in individuals with concomitant heart failure or CKD and in those being treated with a complex drug regimen, including renin-angiotensin system pathway blockers. Kem, David C., et al. “Circadian rhythm of plasma aldosterone concentration in patients with primary aldosteronism.” The Journal of clinical investigation 52.9 (1973): 2272-2277.

Kovesdy, C. P. (2017); Young, W. F. (2007). MR blockers also have the additional property of interfering with the short feedback (paracrine) loop in the adrenal glands, resulting in substantial increases in aldosterone production that may signal via non-genomic pathways in vascular smooth muscle cells and potentially drive adverse cardiovascular consequences. Yin, L. et al. (2012); Brown, J. M. et al. (2020). Osilodrostat is the only ASI to complete mid-stage clinical development to date. In individuals with hyperaldosteronism, osilodrostat produced a modest reduction in blood pressure that was hampered by insufficient selectivity for aldosterone synthesis vis-à-vis cortisol. This resulted in accumulation of the active intermediate 11-deoxycorticosterone (11-DOC), which likely replaced aldosterone as the major MR agonist, thus reducing potential benefit. Schumacher, C. D. et al. (2013). Based on that finding, development was terminated and, until recently, no other ASI with sufficient selectivity and an otherwise favorable pharmacokinetic and safety profile had been developed. However, because of this persistent unmet medical need, interest in developing ASIs for the treatment of hypertension and the reduction of associated cardiovascular complications remains high.

Aldosterone synthase (CYP11B2) is a mitochondrial cytochrome P450 (CYP) enzyme that converts 11-DOC to aldosterone in 3 consecutive steps: 11-DOC converts to corticosterone, which converts to 11-hydroxy-corticosterone, which converts to aldosterone. Yin, L. et al. (2012). CYP11B1, a key enzyme in glucocorticoid biosynthesis, has a high homology to CYP11B2 (>93%). High selectivity for CYP11B2 over CYP11B1 is an essential characteristic for a successful ASI. Hartmann, R. et al. (2003). Potentially important is the ability to restore key aspects of systems homeostasis, including the potential for periods of incomplete aldosterone synthase inhibition during each day to allow for metabolism and clearance of 11-DOC and to reduce the potential for confounding hyperkalemia. The development of improved and more selective inhibitors of CYP11B2 that are suitable for human use has proven to be challenging, with only 2 drugs recently reaching clinical-stage testing. One of these drugs, Compound A, described in this report, is a novel synthetic, orally administered, non-peptide small molecule that is a highly selective inhibitor of CYP11B2. In healthy human volunteers, Compound A demonstrated optimized pharmacokinetic (PK) and pharmacodynamic (PD) evidence of inhibition of renal tubular aldosterone signaling across a broad dose range with no suppression of basal or stimulated cortisol production. Based on these findings, Compound A has been advanced to a novel, targeted Phase II trial in individuals with hypertension and evidence of autonomous aldosterone overproduction. Trial on the Safety and Efficacy of Compound A in Patients with Uncontrolled Hypertension (Target-HTN). ClinicalTrials.gov identifier: NCT05001945.

### Compound A

Compound A is a potent inhibitor of hCYP11B2 and has minimal inhibition of hCYP11B1 (1.27 nmol/L vs 475 nmol/L, respectively) at projected clinical doses. The approximately 374-fold selectivity of Compound A for hCYP11B2 vs hCYP11B1 was much greater than the 3.6-fold selectivity observed with the reference compound osilodrostat. In animal studies, single oral administration of the free base of Compound A significantly decreased PAC in a sodium-depleted monkey model, whereas PCCs were not affected in ACTH-loaded monkeys, demonstrating the highly potent and selective inhibition of CYP11B2 by Compound A in vivo. Findings from the toxicology studies were considered to be related to its pharmacologic activity.

Administrations of Compound A in SAD and MAD portions were well-tolerated at single doses up to 800 mg under fasting conditions and once-daily dosing up to 360 mg under fed conditions in healthy men. Compound A was well-tolerated by healthy women and men aged >65 years. The sex and age of participants had no significant impact on the PK of Compound A.

Statistically significant reductions in PAC AUC₀₋₂₄ were observed after administration of single doses of Compound A compared with placebo. Reductions were observed from 2 through at least 12 hours post-dose and sustained in a dose-dependent manner up to 24 hours post-dose. PAC (AUC₀₋₂₄) was reduced by up to 40% with single 100- to 200-mg doses and up to 70% with single 400- to 800-mg doses. In contrast to significant reductions in aldosterone, no meaningful effect on serum cortisol levels was observed with any of the doses tested. The lack of effect of Compound A on serum cortisol levels was also seen in the MAD part of the study (part 2), both at baseline and in response to ACTH stimulation. The changes in PAC were similar between male and female participants, as well as between men aged ≥65 years vs <65 years.

Measurement of the effect of Compound A on renal sodium excretion provides a direct measure of the effect of aldosterone on renal tubular function, and thus on the potential to reduce intravascular volume and ameliorate volume-dependent systemic hypertension. In the SAD portion of the study, Compound A doses of ≥10 mg produced an increase in urinary log₁₀ (10×Na⁺/K⁺) ratio, confirming that the observed reduction in aldosterone had the anticipated functional effect—at least during the initial suppression of aldosterone production. Measurements of PAC demonstrated a dose-dependent effect on the duration of aldosterone suppression. In the MAD portion of the study, Compound A doses of 40, 120, and 360 mg showed maximum PAC suppression was maintained for approximately 12 hours.

Although the urinary electrolyte measurements confirmed that there was brisk onset of natriuresis at all doses tested, the duration and stability of the sodium depletion is not addressed by that measurement. It is indeed possible that if suppression of aldosterone is too brief, there could be compensatory sodium retention later in the day, with accompanying kaliuresis and no sustained effect on intravascular volume and blood pressure. However, measurements of serum potassium—and to a lesser extent sodium—help to address this issue. Within 2 days of initiation of Compound A treatment, serum potassium rose in all 3 dose cohorts (part 2) by approximately 0.5 mmol/L and remained elevated throughout the remainder of the 7 days of treatment. Upon cessation of Compound A treatment, serum potassium levels rapidly fell toward pretreatment baseline. Had the duration of aldosterone suppression been inadequate to maintain renal tubular effects, then one would have anticipated that there would not have been maintenance of elevated serum potassium (and the modest reduction in serum sodium), particularly in the lower dose cohort. This provides strong PD evidence that the duration of suppression of aldosterone production was sufficient to maintain a state of sodium and volume depletion.

There is a typical diurnal pattern of aldosterone secretion leading to a spike in PAC starting in the pre-waking hours followed by a progressive decline as the day progresses. Thus, suppression of daytime aldosterone production, even with absent or incomplete suppression during sleep, would be anticipated to have a significant and sustained effect on downstream biological effects, including electrolyte and volume homeostasis. Single 100-mg doses of Compound A and multiple 120-mg doses of Compound A allowed for the return of baseline aldosterone levels by 16 hours (FIG. 10 to FIG. 12). This is important because complete suppression of aldosterone can produce a persistent state of hyperkalemia and mild non-anion gap metabolic acidosis. Szylman, P. et al. (1976); Harris, A. N. et al. (2018).

Hyperkalemia is known to be an issue with the MR antagonist spironolactone, which has a complex set of active metabolites, some of which have half-lives in excess of 24 hours. Spironolactone is associated with up to an 10% risk of hyperkalemia and is used relatively infrequently despite being a very effective antihypertensive agent. Once-daily dosing of Compound A without substantial accumulation is anticipated to be an important determinant of safety. In the event that hyperkalemia does develop, cessation of Compound A dosing should allow for rapid resolution of hyperkalemia.

One of the limiting factors for osilodrostat was excessive accumulation of 11-DOC. Because 11-DOC is itself a MR agonist, its accumulation can oppose the potential benefit of aldosterone synthesis inhibition. In the MAD part of the study, Compound A was observed to increase serum levels of 11-DOC. However, the accumulation was relatively insignificant in the 40- and 120-mg daily cohorts and did not exceed the normal range. In contrast, the 360-mg daily dose cohort showed a much more dramatic increase in 11-DOC, to a level that might be anticipated to be problematic. Given that the maximum PD effects on renal electrolyte regulation had a relatively flat dose-response that was near maximum at 40 mg daily, the 11-DOC accumulation data suggest that the dose selection for the treatment of hypertension should avoid the higher dose level and focus on doses up to 120 mg daily.

Collectively, the results of this series of pharmacology studies indicated that Compound A was a selective inhibitor of hCYP11B2 and was not associated with any AEs in the central nervous, respiratory, or cardiovascular systems.

### Perspectives

The development of improved and more selective inhibitors of CYP11B2 suitable for human use has proven to be challenging. The preclinical and clinical development of a new, highly selective hCYP11B2 inhibitor, Compound A, has demonstrated a favorable safety profile in non-human primates and healthy human participants. In healthy human participants, Compound A demonstrated optimized PK and PD evidence of inhibition of renal tubular aldosterone signaling across a broad dose range with no suppression of basal or stimulated cortisol production. Based on these findings, Compound A has been advanced to a novel, targeted phase 2 trial in individuals with hypertension and evidence of autonomous aldosterone overproduction. Monticone, S. et al. (2017).

### Pathophysiological Novelty and Relevance:

Compound A is a novel CYP 11β2 beta hydroxylase inhibitor that
(a) is selective for CYP 11β2 beta hydroxylase over CYP 11β1 beta hydroxylase
(b) produces clinically meaningful reductions in aldosterone
(c) with daily dosing allows for the return of baseline aldosterone levels at ˜16 hours.

The reduction of PAC in patients with classical hyperaldosteronism, as well as obesity-associated inappropriate aldosterone production, is an important component in addressing treatment-resistant hypertension. The major risk of complete suppression of PAC is hyperkalemia which may potentially be avoided by the PK/PD profile of daily dosing of Compound A.

### Example 4

A randomized, double-blinded, placebo-controlled, dose-ranging, multicenter study was conducted to evaluate the effect of orally administered Compound A HBr on blood pressure for the treatment of hypertension in male and female subjects ≥18 years of age.

### Study Design

The study consists of two parts. For enrollment into Part 1 of the study, a subject's value of plasma renin activity (PRA) must be ≤1 ng/mL/h based on morning measurement. If the value of PRA >1 ng/mL/h based on morning measurement, then subjects may be eligible to enter Part 2 of the study.

For Part 1, 163 enrolled subjects ≥18 years of age were randomized into 6 equal treatment groups (1:1:1:1:1:1) to 12.5 mg BID, 25 mg BID, 12.5 mg QD, 50 mg QD, 100 mg QD, or placebo. After a review of interim clinical data, the 2 lowest dose levels (12.5 mg QD and 12.5 mg BID) were stopped for future randomization due to a lack of consistent meaningful reduction of blood pressure, but the patients randomized to that point remained in the study through completion. Thus, after the review of interim clinical data, subjects were randomized into 4 equal treatment groups (1:1:1:1) to 25 mg BID, 50 mg QD, 100 mg QD, or placebo.

For Part 2, 36 enrolled subjects ≥18 years of age were randomized (5:1) to either 100 mg QD Compound A HBr or placebo such that the Compound A HBr treatment group consisted of approximately 30 subjects and the placebo treatment group will consist of approximately 6 subjects.

Subjects orally administered the assigned study drug (Compound A HBr or placebo) according to the assigned dosing regimen for 8 weeks beginning on Study Day 1. All subjects in Part 1 (regardless of dosing group) received BID dosing to preserve the integrity of the blind; active drug is administered as the morning dose for all QD dose groups. Subjects returned to the research facility or were seen by the clinical investigator or approved home health care professional at the end of Study Weeks 1, 2, 3, 4, 5, 6, 7, and 8 (±2 days) for protocol-defined efficacy and safety assessments and procedures, assessment of adverse events (AEs), and confirmation of compliance with study drug usage. Subjects also completed a telephone visit and blood pressure (BP) check at home approximately 3 days post last dose of study drug. Subjects attended up to 14 full clinic visits, including a pre-Screening visit, a Screening/start of Placebo Run-in visit, a second visit during Placebo Run-in, a clinic visit to initiate the ABPM procedure, a Randomization visit, 8 weekly visits during double-blind treatment, and an end-of-study visit scheduled 4 weeks after the last study treatment for final efficacy and safety assessments.

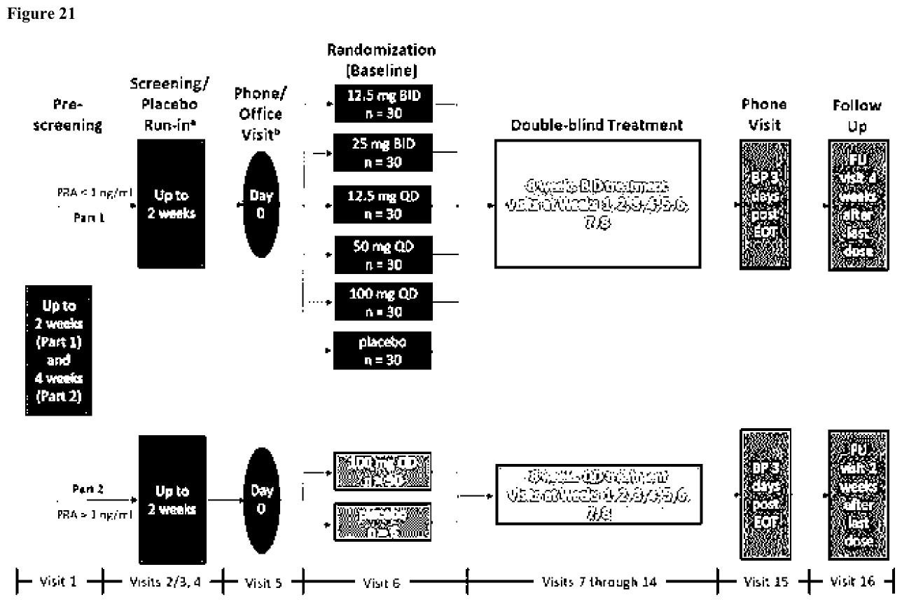

A schematic of the study design is shown in FIG. 21.

### Automated Office Blood Pressure (AOBP) Procedure

An automated oscillometric sphygmomanometer device was used to measure the subjects' systolic and diastolic blood pressure in office after approximately 5 minutes of rest in the seated position.

### 24-Hour Ambulatory Blood Pressure Monitoring (ABPM) Procedure

Ambulatory blood pressure monitoring was accomplished with an ambulatory blood pressure monitoring device that consists of a blood pressure cuff worn on a subject's arm attached to a small recording device that is typically attached to the subject's belt or waistband.

The ABPM device is worn for 24 hours. Throughout that period, the device records the subject's blood pressure at regular intervals, during the subject's routine daily activities and while they are sleeping. The ABPM thus provides a complete record of the subject's blood pressure over a 24-hour period.

24-hour ABPM was measured in the clinic at baseline and Study Week 7. If, for any reason, the ABPM procedure was deemed a failure at the end of Study Week 7, it may have been repeated at Study Week 8 and therefore no imputation was employed regardless of use of rescue medications. Additionally, ABPM was also collected at the end of Study Week 4 in Part 2. If a repeat test was performed, it supersedes the original test results for that visit.

Specific derived variables based on ABPM measurements include mean 24-hour, mean Daytime, and mean Nighttime of SBP, DBP, and heart rate.

Change from baseline to Week 7 in 24-hour mean SBP (and DBP) based on ABPM will be analyzed using an ANCOVA with a term for treatment group and a baseline mean 24-hour value as a covariate.

The nighttime dip is defined as

100%×(mean daytime SBP−mean nighttime SBP)/mean daytime SBP

It is expressed as a percentage and is summarized by treatment group and visit using descriptive statistics. In addition, the number and percentage of subjects with nighttime dip in each of the dipper categories (Bloomfield & Park, 2015) is presented by treatment group and visit. The categories are:
(a) <10%
(b) 10-20% inclusive
(c) >20%

### Eligibility Criteria

### Inclusion Criteria

The study was conducted using subjects meeting the following inclusion criteria:
(a) Male and nonpregnant, nonlactating female subjects ≥18 years of age.
(b) Automated office blood pressure (AOBP) with systolic blood pressure (SBP) ≥130 mm Hg
(c) Background antihypertensive treatment of ≥2 drugs
(d) Serum cortisol ≥18 mcg/dL

### Exclusion Criteria

Subjects were excluded from the study if they met any of the following exclusion criteria:
(a) Concomitant use of epithelial sodium channel inhibitors or mineralocorticoid receptor antagonists
(b) Subjects with hypokalemia
(c) Subjects with hyperkalemia
(d) Subjects with serum cortisol <3 mcg/dL
(e) Subjects with serum sodium <135 mEq/L
(f) Subjects with estimated glomerular filtration rate <60 mL/min/1.73 m2
(g) Subjects with type 1 or uncontrolled (hemoglobin A1c ≥9%) type 2 diabetes mellitus
(h) Subjects with body mass index >40 kg/m²
(i) Subjects with unstable angina
(j) Subjects with SBP ≥175 mm Hg or diastolic blood pressure (DBP) ≥100 mm Hg for Part 1 and SBP ≥160 mm Hg or DBP ≥100 mm Hg for Part 2 at Pre-Screening, Screening/Start of Placebo Run-in, or Randomization
(k) Subjects with a decrease in SBP ≥20 mm Hg or DBP ≥10 mm Hg from sitting to standing position at screening
(l) Subjects who, in the opinion of the investigator, have suspected nonadherence to antihypertensive treatment
(m) Subjects who, in the opinion of the investigator, have any major medical illness or symptoms
(n) Subjects who, in the opinion of the investigator, have any acute or chronic medical or psychiatric condition
(o) Subjects undergoing treatment with any of the following medications:
   (i) Topical corticoids
   (ii) Sympathomimetic decongestants
   (iii) Theophylline
   (iv) Phosphodiesterase type 5 inhibitors
   (v) NSAIDs
   (vi) Intramuscular steroids
   (vii) Estrogen
   (viii) Cytochromes
   (ix) Strong CYP3A and CYP3A4 inducers
(p) Subjects with known hypersensitivity to Compound A HBr or any of the excipients
(q) Subjects who are night-shift workers

### Arms and Interventions

The study contained the following arms and corresponding interventions:

TABLE 7

| Arm | Intervention* |
|---|---|
| Placebo Comparator: Placebo (Part I) | Other: Placebo (Part I) |
| Placebo tablet(s) by mouth once or twice | Placebo tablet(s) by mouth once or twice |
| daily. | daily. |
| Experimental: Dose 1 (Part I) | Drug: Compound A HBr (Part I) |
| Compound A HBr tablet(s) by mouth once | Tablet(s) containing 12.5 mg of Compound |
| daily. | A HBr by mouth once daily. |
| Experimental: Dose 2 (Part I) | Drug: Compound A HBr (Part I) |
| Compound A HBr tablet(s) by mouth twice | Tablet(s) containing 12.5 mg of Compound |
| daily. | A HBr by mouth twice daily. |
| Experimental: Dose 3 (Part I) | Drug: Compound A HBr (Part I) |
| Compound A HBr tablet(s) by mouth twice | Tablet(s) containing 25 mg of Compound A |
| daily. | HBr by mouth twice daily. |
| Experimental: Dose 4 (Part I) | Drug: Compound A HBr (Part I) |
| Compound A HBr tablet(s) by mouth once | Tablet(s) containing 50 mg of Compound A |
| daily. | HBr once daily. |
| Experimental: Dose 5 (Part I) | Drug: Compound A HBr (Part I) |
| Compound A HBr tablet(s) by mouth once | Tablet(s) containing 100 mg of Compound |
| daily. | A HBr once daily. |
| Placebo Comparator: Placebo (Part II) | Other: Placebo (Part II) |
| Placebo tablet(s) by mouth once daily. | Placebo tablet(s) by mouth once daily. |
| Experimental: Dose (Part II) | Drug: Compound A HBr (Part II) |
| Compound A HBr tablet(s) by mouth once | Tablet(s) containing 100 mg of Compound |
| daily. | A HBr by mouth once daily. |

### Study Endpoints

### Primary Endpoint

The primary endpoint is the change in office-measured (mean of last 2 of 5 unattended measurements using an automated oscillometric sphygmomanometer device after approximately 5 minutes of rest in the seated position) systolic blood pressure (SBP) from baseline to the end of Study Week 8.

### Secondary Endpoint

Secondary endpoints of this study were:
(a) Change in 24-hour ambulatory blood pressure monitoring (ABPM) parameters (systolic and diastolic) from baseline to the end of Study Week 7.
(b) Change in office-measured SBP from baseline to the end of Study Weeks 1, 2, 3, 4, 5, 6, and 7.
(c) Change in office-measured diastolic blood pressure (DBP) from baseline to the end of Study Weeks 1, 2, 3, 4, 5, 6, 7, and 8.
(d) Proportion of subjects who achieve office-measured BP of ≤130/80 mm Hg by the end of Study Week 8.

### Pharmacodynamic Endpoints

Pharmacodynamics endpoints of this study were:
(a) Change in plasma 11-deoxycortisol and PRA from baseline to the end of Study Week 4 and to end of follow up (i.e. end of Study Week 12 for Part 1 and end of Study 10 for Part 2).
(b) Change in serum aldosterone, cortisol, and 11-deoxycorticosterone concentration from baseline to the end of Study Week 4 and to end of follow-up.

### Pharmacokinetic Endpoints

Pharmacokinetic endpoints of this study are PK parameters, including, of area under the plasma concentration versus time curve (AUC), maximum plasma concentration (Cmax), time to maximum concentration (Tmax), and half-life (t_{1/2}) will be summarized descriptively for Randomization (baseline) and Study Weeks 1, 4, and 8

### Safety Endpoints

Safety endpoints of this study are:
(a) Incidence and severity of all spontaneously reported adverse events (AEs)
(b) Changes in vital signs (standing SBP, standing DBP, body temperature, heart rate, and respiratory rate)
(c) Changes in electrocardiogram parameters (including cardiac intervals: PR, QRS, QT, and corrected QT interval using Fridericia's formula)
(d) Changes in clinical laboratory assessments (hematology, chemistry, coagulation, and urinalysis)
(e) Change in office-measured SBP from Study Week 8 (end-of-treatment period to end of follow up (i.e. end of Study Week 12 for Part 1 and end of Study 10 for Part 2).

### Analysis Methodology

The following analysis sets are defined in this study:

### Full Analysis Set (FAS)

The FAS includes all randomized subjects who have received at least 1 dose of randomized study treatment (MLS-101 or placebo). The FAS will be the primary set for efficacy analyses. In analyses performed on the FAS, unless otherwise specified, subjects will be analyzed according to the randomized study treatment group.

### Per Protocol Set (PPS or PP)

The Per Protocol Set includes all subjects in the FAS who have completed the Study Week 8 visit without any major protocol violations that could influence the validity of the data for the primary efficacy evaluations. In the analyses based on PPS, subjects will be analyzed according to the randomized study treatment group. All criteria to exclude subjects from the PPS will be made based on a blinded review of the data prior to the unblinding of the study.

A subject may be excluded from the Per Protocol Analysis Set if any of the following criteria are met:
(a) Not meeting Inclusion/Exclusion criteria
(b) Use of prohibited medications. Subjects using rescue medications will not be excluded from the PPS unless subjects have met other criteria excluding them from the PPS
(c) Not compliant with the study drug
(d) Out of window efficacy assessment at study Week 8 visit

Alternate criteria for exclusion from the Per Protocol Analysis Set were also applied to accommodate unforeseen events that occurred during the conduct of the study.

Analyses on Per Protocol Analysis Set will be of supportive purpose and limited to primary endpoint (i.e., “product Estimand”).

### Safety Analysis Set (SAF)

The Safety Analysis Set includes all enrolled subjects who received at least one dose of study treatment (MLS-101 or placebo). In analyses performed on the Safety Analysis Set, subjects will be analyzed according to their actual treatment received.

### PK/PD Analysis Set (PKPD)

PK/PD Analysis Set includes all subjects in the SAF who have sufficient data available for the analysis of pharmacokinetic and pharmacodynamic measurements. In the analyses based on PKPD, subjects will be analyzed according to the actual treatment received.

### Definition of Baseline

Baseline is defined as the last available observed value of the parameter of interest prior to the first administration of the investigational medicinal product (IMP) for the double-blinded treatment period.

For AOBP measurements and any other clinical or laboratory variable for which there are replicate evaluations at the screening and baseline visits, baseline is defined as the mean of the last two non-missing values prior to first administration of the IMP for the double-blinded treatment period.

Change from baseline is calculated as: post-baseline result−baseline result.

Percentage change from baseline is calculated as: (change from baseline/baseline result)×100%.

### Summary of Results

There was a dose-response relationship across the QD dose range, with doses of 50 mg and 100 mg QD associated with a mean reduction in AOBP measured systolic BP of −11 to −13 mmHg (per-protocol, placebo-corrected, analysis of Part-1 100 mg cohort=−10.3 mmHg, Full-analysis set combining Part-1 and Part-2 (interim)=−9.9 mmHg, N=58 active).

Once daily dosing was as effective as twice daily dosing, with results in the two BID cohorts no better than those in the 50 mg and 100 mg QD cohorts.

In a pooled analysis of 50 mg QD, 100 mg QD, 12.5 mg BID and 25 mg BID cohorts (N=103) 25% of subjects demonstrated a change in BPsys >−25 mmHg and 41% a change >−15 mmHg.

Using automated office blook pressure (AOBP), there was little difference in treatment response between individuals in Part 1 and available results from Part 2, suggesting PRA does not appear to be a strong determinant of response (serum and urine aldosterone were also uninformative).

24 h ambulatory blood pressure measurements demonstrated overnight reduction of systolic BP of −11.5+/−2.9 mmHg in the 100 mg QD cohort, with an associated increase in nigh-time “BP-dipping”, consistent with persistent nocturnal benefit after morning dosing,

The pooled (Part 1 and Part 2) 100 mg QD safety set (n=60) showed good safety and tolerability, with no effects on serum cortisol, few episodes of mild or moderate hyperkalemia and no episodes of severe hyperkalemia.

### Automated Office Blood Pressure Results

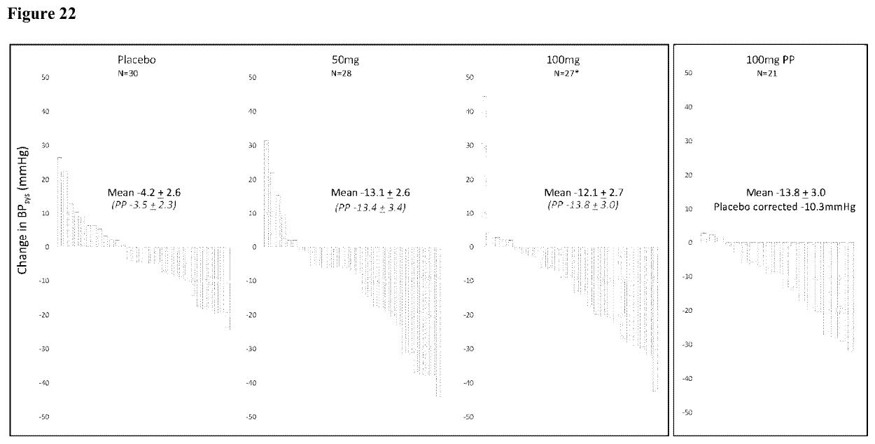

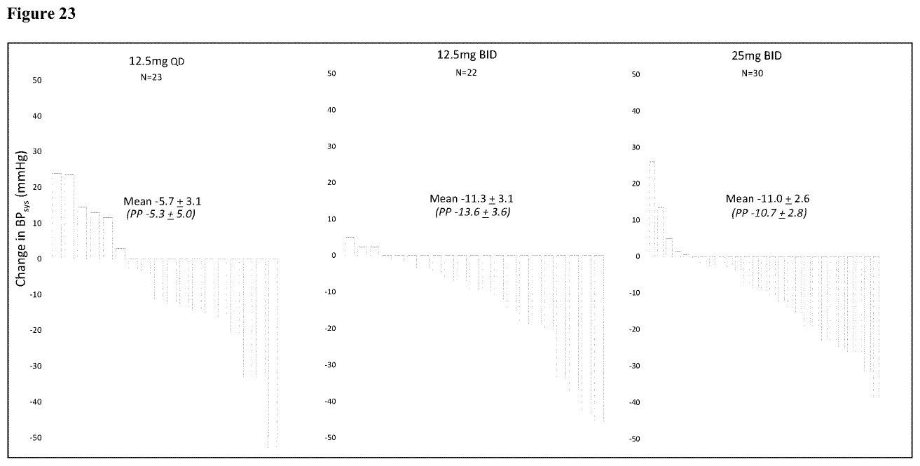

A full analysis and safety set (FAS) analysis was conducted using all subjects with week 8 measurement. A per-protocol (PP) analysis was also conducted using all subjects completing treatment through the eighth week visit. Waterfall plots showing the AOBP change in systolic blood pressure at week 8 from the FAS analysis of placebo, 50 mg QD and 100 mg QD groups and the PP analysis of the 100 mg group is provided in FIG. 22. Waterfall plots showing the AOBP change in systolic blood pressure at week 8 from the FAS analysis of 12.5 mg QD, 12.5 mg BID, and 25 mg BID groups is provided in FIG. 23. A modeled mean and per-protocol observed mean are also shown for each group.

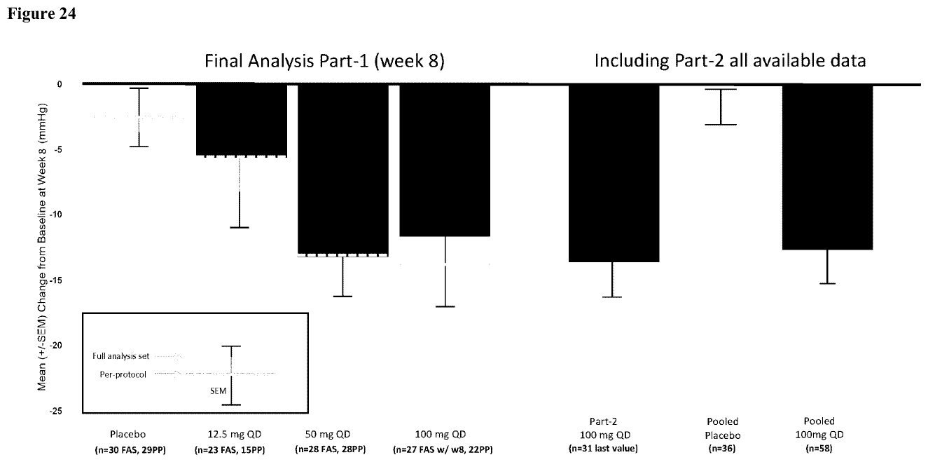

Mean change in systolic blood pressure from baseline is shown in FIG. 24. The figure provides a final analysis including both full analysis set (FAS, all evaluable subjects receiving at least one dose of Compound A HBr) and per-protocol (PP, only those receiving ≥75% of study drug with week 8 visit). Part 2 data shows the interim average of last visit week 5-6.

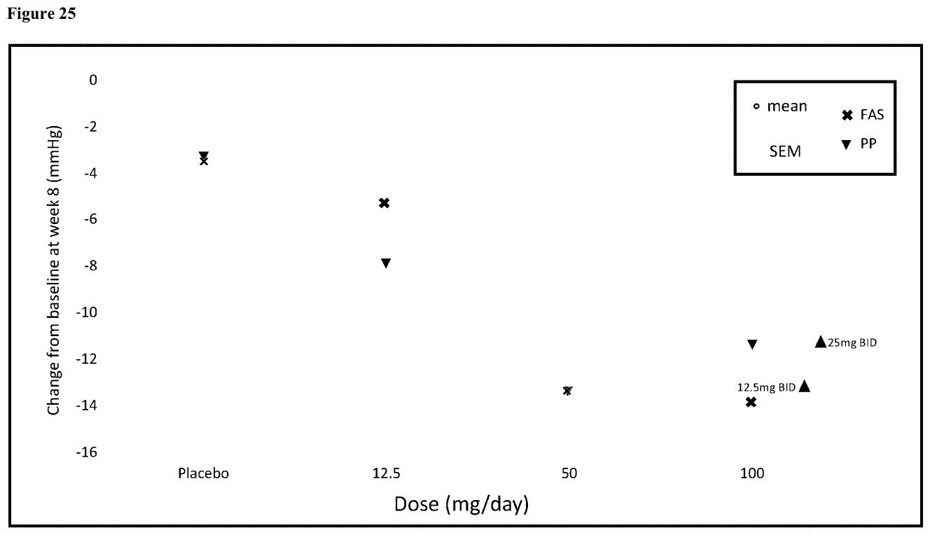

A Compound A HBr dose-response was observed based on analysis of systolic AOBP change from baseline among the QD regimens. FIG. 25 shows the mean observed automated office blood-pressure change from baseline at week 8 for QD dosing regimens. BID per-protocol cohorts are shown on the far right of this graph.

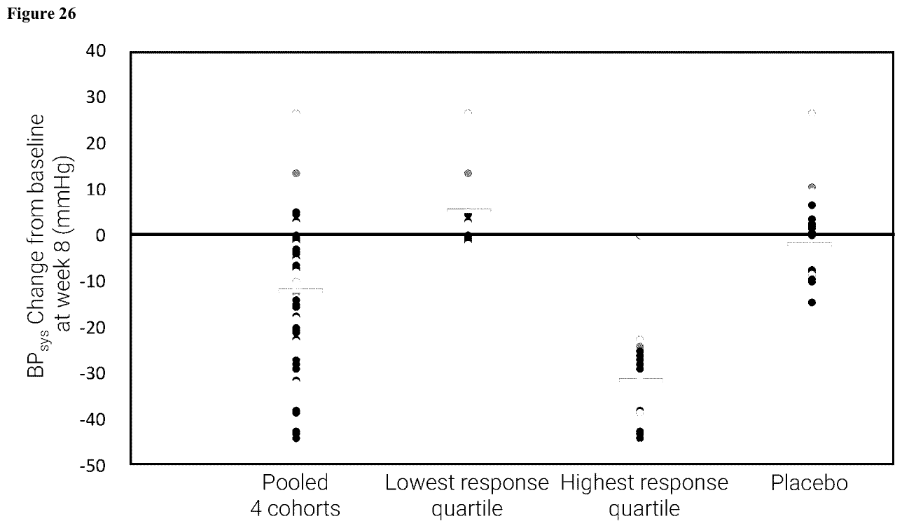

An analysis was conducted in which the change in systolic blood pressure from baseline at week 8 were pooled for the 50 mg QD, 100 mg QD, 12.5 mg BID, and 25 mg BID cohorts and then separated into quartiles based on degree of systolic blood pressure response. FIG. 26 is a graph showing the systolic blood pressure change from baseline at week 8 for the pooled cohorts, the lowest response quartile, the highest response quartile, and placebo. 25% of subjects achieved >−23 mmHg fall in systolic blood pressure, with mean reduction of −33.4+1.5 mmgHg. 41% of subjects achieved a ≥15 mmHg fall in systolic blood pressure.

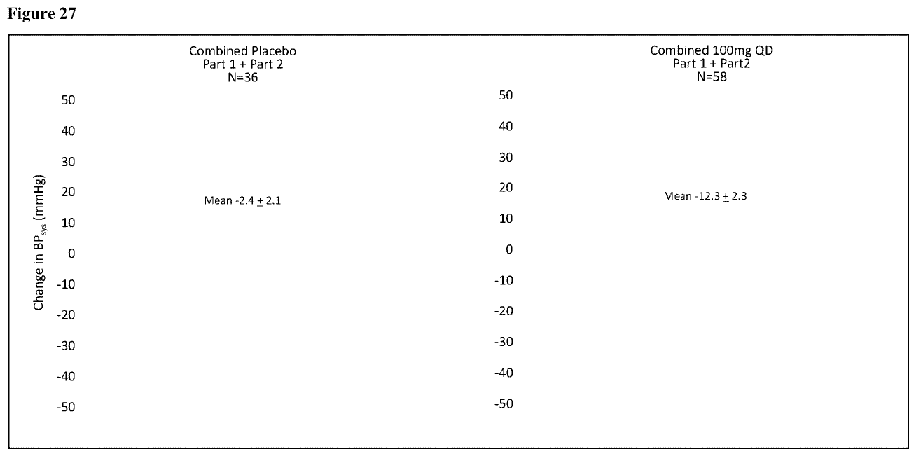

FIG. 27 is a waterfall plot showing change in systolic blood pressure from placebo and 100 mg QD groups pooled from both Parts 1 and 2. Part 2 data from interim snapshot with all subjects randomized and average last visit week 5-6, minimum week 2.

### Analysis of Factors Affecting Change in Blood Pressure

An analysis was conducted to identify factors affecting the degree of blood pressure reduction in hypertensive subjects and is summarized in the table below.

TABLE 8

| Difference from placebo | | | | | 12.5 mg | |
|---|---|---|---|---|---|---|
| (* p < 0.05) | | 12.5 mg QD | 50 mg QD | 100 mg QD | BID | 25 mg BID |
| Gender (N) | Male (N) | −2.3 (11) | −8.3 (13) | −12.4 (12) | −13.8 (8) | −9.5 (11) |
| | Female (N) | −0.2 (12) | −11.3 (15) | −5.1 (18) | −3.79 (14) | −5.18 (19) |
| Age (N) | <65 (N) | −0.3 (10) | −10.2 (13) | −15.7 (8) | −5.4 (6) | −8.5 (13) |
| | 65-79 (N) | 1.1 (10) | −8.8 (13) | −1.6 (18) | −1.5 (13) | −2.4 (15) |
| Race (N) | Black (N) | 6.75 (11) | −6.9 (8) | −7.0 (15) | 5.57 (7) | −10.1 (7) |
| | Other (N) | −7.5 (12) | −12.1 (20) | −9.2 (15) | −14.7 (15) | −7.2 (23) |
| Baseline | Low tertile | −10.7 | −8.3 | −11.8 | 3.8 | −8.8 |
| BPsys | Middle tertile | 9.3 | −11.9 | −11.8 | −9.7 | −4.0 |
| | High tertile | −3.6 | −20.6* | −9.7 | −9.7 | −5.6 |
| Background | 2 medications | 1.8 (14) | −3.7 (20) | −7.5 (14) | −1.2 (7) | 1.4 (14) |
| hypertension | (N)** | | | | | |
| therapy | 3+ medications (N) | −6.0 (9) | −19.1* (8) | −9.6 (16) | −12.2* (15) | −15.8* (16) |

All values determined using least squares analysis of modeled means using all available information.
**Difference in effect between 2 and 3+ background due to an imbalance in placebo response.

### Ambulatory Blood Pressure (ABPM) Results

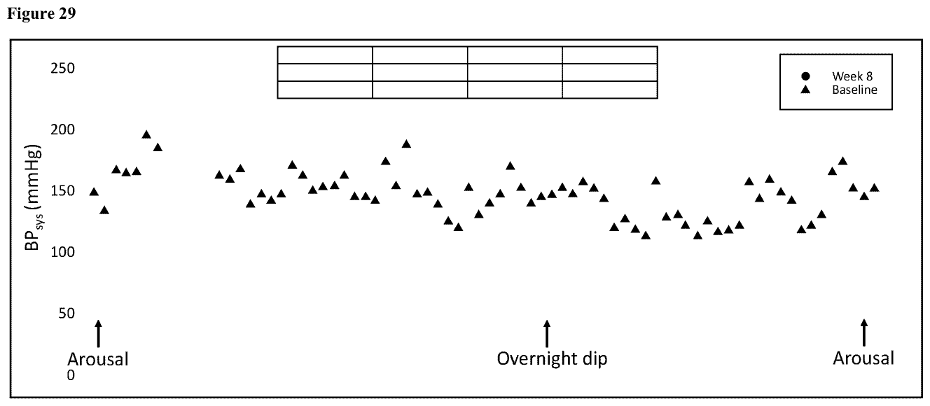

A graph showing an example of ambulatory 24-hour blood pressure monitoring is providing in FIG. 29. The graph shows the 24-hour ambulatory blood pressure (systolic) of a single subject receiving Compound A HBr 100 mg QD versus baseline, showing an average 24-hour blood pressure reduction and restoration of normal nocturnal dipping pattern.

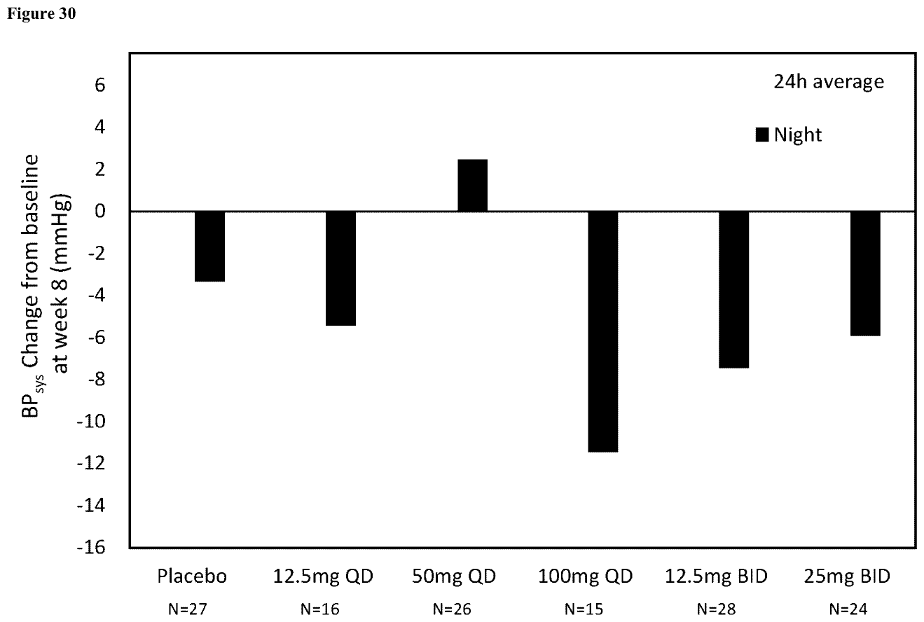

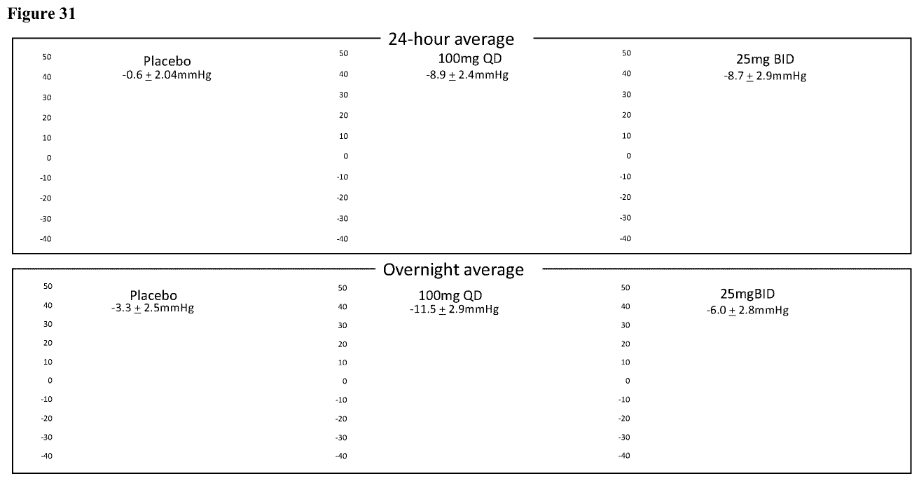

A graph showing the change in systolic blood pressure at week 8 relative to baseline as measured using the ABPM full analysis set is provided in FIG. 30. Waterfall plots showing the 24-hour average and overnight average ABPM change at 8 weeks relative to baseline is provided in FIG. 31. 100 mg QD dose levels provide excellent 24-hour blood pressure reduction. Overnight blood pressure reduction from the 100 mg QD dose level appears to be superior to 25 mg BID.

As shown in the summary table below, the most consistent benefit across all metrics was observed in the 100 mg QD cohort.

TABLE 9
Change in BP mmHg Week 8, Full Analysis Set

| Mean (sem) | | Placebo | 12.5 mg QD | 50 mg QD | 100 mg QD | 12.5 mg BID | 25 mg BID |
|---|---|---|---|---|---|---|---|
| AOBP | N | 30 | 22 | 28 | 30 | 22 | 30 |
| | BP_{sys} | −4.3 (2.6) | −5.7 (3.1) | −13.9 (2.6) | −12.2 (2.7) | −11.3 (3.1) | −11.0 (2.6) |
| | BP_{dias} | −1.3 (1.8) | −6.1 (1.8) | −8.1 (1.7) | −4.76 (1.5) | −6.1 (1.8) | −3.83 (2.1) |
| ABPM | N | 30 | 23 | 28 | 30 | 16 | 30 |
| ABPM | BP_{sys} | −0.6 (1.9) | −5.2 (3.4) | −1.8 (3.0) | −8.9 (2.2) | −5.7 (3.2) | −8.7 (2.9) |
| (24 h) | BP_{dias} | −1.4 (1.0) | −2.3 (1.7) | −3.0 (1.6) | −5.9 (1.5) | −5.4 (2.2) | −6.1 (1.8) |
| | BP_{mcan} | −1.0 (1.4) | −3.7 (2.6) | −2.4 (2.2) | −7.4 (1.8) | −5.5 (2.7) | −7.4 (2.3) |
| ABPM | BP_{sys} | −3.3 (2.3) | −5.4 (4.2) | 2.4 (4.2) | −11.5 (2.5) | −7.5 (4.0) | −6.0 (2.8) |
| (night) | | | | | | | |
| Dip | <10% | 63 | 64 | 78 | 46 | 69 | 79 |
| (%) | 10- | 37 | 36 | 21 | 46 | 31 | 17 |
| | 20% | | | | | | |
| | >20% | 0 | 0 | 0 | 8 | 0 | 4 |

$$Night - time dip defined as 100 \% \times ( 24 - hour ambulatory Daytime SBP - 24 - hour ambulatory monitoring Night - time SBP ) / 24 - hour ambulatory monitoring Daytime$$

### Safety

No serious adverse events (SAEs) related to the study medication were observed in the trial. Adverse events requiring drug discontinuation or dose reduction, showing incidence in Part 1 versus incidence Part 2 is provided in Table 10 below.

TABLE 10

| | | Total | Total | Part 1 | Part 1 | Part 2 | 100 mg QD |
|---|---|---|---|---|---|---|---|
| | | Placebo | active | All active | 100 mg QD | 100 mg QD | Pooled |
| | N | 35 | 190 | 159 | 29 | 31 | 60 |
| Study | Hypotension | 0 | 3 (1.6%) | 2 (1.2%) | 1 (3.4%) | 1 (2.8%) | 2 (3.3%) |
| drug | Hyponatremia | 0 | 1 (0.5%) | 0 | 0 | 1 (3.4%) | 1 (1.7%) |
| discontinuation | Hyperkalemia | 0 | 6 (3.2%) | 6 (3.8%) | 2 (6.9%) | 0 | 2 (3.3%) |
| | Other possibly | 0 | 1 (0.5%) | 1 (0.6%) | 1 (3.4%) | 0 | 1 (1.7%) |
| | related | | | | | | |
| | Other | 0 | 12 (6.3%) | 11 (6.9%) | 3 (6.0%) | 1 (2.8%) | 4 (6.7%) |
| | unrelated | | | | | | |
| Dose | Hyperkalemia | 0 | 7 (4.1%) | 6 (3.8%) | 1 (3.4%) | 1 (3.2%) | 2 (3.3%) |
| [symbol] titrati [symbol] | Hyponatremia | 0 | 1 (0.5%) | 0 | 0 | 1 (3.2%) | 1 (1.7%) |
| | Other possibly | 0 | 0 | 0 | 0 | 0 | 0 |
| | related | | | | | | |
| | Other | 1 (1.4%) | 1 (0.5%) | 1 (0.6%) | 0 | 0 | 0 |
| | unrelated | | | | | | |

[symbol]

indicates data missing or illegible when filed

### Serum Potassium

Change in group mean serum potassium (K+) is shown in the table below.

TABLE 11

| | Part 1 | Part 2 | Pooled |
|---|---|---|---|
| Mean (mMol/L) | +0.50 | +0.23 | +0.35 |
| SD | 0.96 | 0.51 | 0.75 |
| N | 26* | 31 | 57 |
| SEM | 0.19 | 0.09 | 0.10 |

Number of individuals in the 100 mg QD cohorts with verified or multiple increased serum potassium above normal range during treatment is shown in the table below.

TABLE 12

| | 5.2-5.5 | 5.6-6.0 | 6.1-6.5 | >6.5 |
|---|---|---|---|---|
| Serum K+ | mMol/L | mMol/L | mMol/L | mMol/L |
| Part 1 | 5 (18.5%) | 3 (10.7%) | 0 | 1* |
| Part 2 | 1 (4%) | 0 | 0 | 0 |
| Pooled | 6 (10.3%) | 3 (5.2%) | 0 | 0 |

*Measure was an isolated incident not verified by repeat measurement with study drug discontinuation (protocol deviation).

Change in Estimated Glomerular Filtration Rate (eGFR)

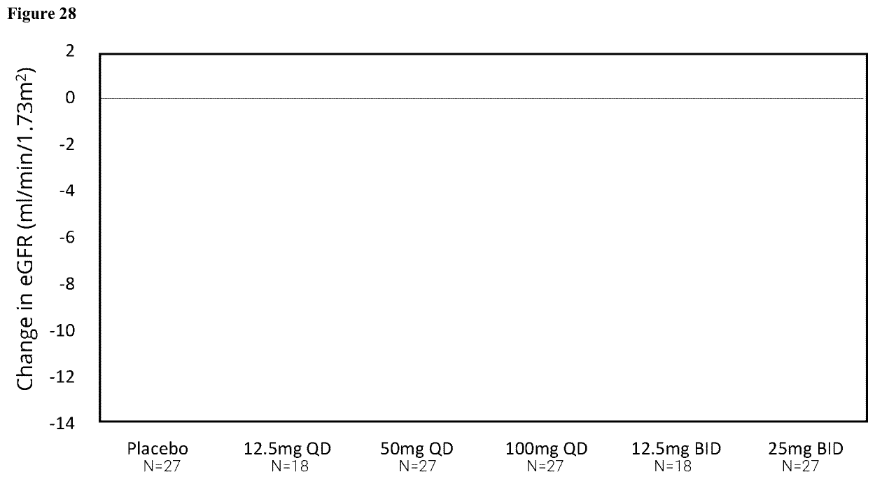

A dose-dependent and reversible reduction in eGFR was observed. This phenomenon has been reported with ACE/ARB and more recently SGLT2 inhibition due to reduced intra-glomerular pressure and felt to attenuate progression of hypertensive nephropathy. A graph showing the change in estimated glomerular filtration rate (eGFR) in different dosing cohorts is provided in FIG. 28.

### BIBLIOGRAPHY

Atlas, S. A. (2007). The renin-angiotensin aldosterone system: pathophysiological role and pharmacologic inhibition. J Manag Care Pharm. 2007; 13(8 Suppl B):9-20.
Brown, J. M. et al. (2020). The unrecognized prevalence of primary aldosteronism. Ann Intern Med. 2020; 173:10-20.
Byrd, J. B. (2015). Aldosterone synthase promoter polymorphism and cardiovascular phenotypes in a large, multiethnic population-based study. J Investig Med. 2015; 63:862-6.
Calhoun, D. A. (2016). Refractory and resistant hypertension: antihypertensive treatment failure versus treatment resistance. Korean Circ J. 2016; 46:593-600.
Calhoun, D. A. et al. (2002). Hyperaldosteronism among black and white subjects with resistant hypertension. Hypertension. 2002; 40:892-6.
Chong, C. et al. (2017). Regulation of Aldosterone Secretion by Mineralocorticoid Receptor-Mediated Signaling. Endocrinol. 2017; 232(3):525-534. doi:10.1530/JOE-16-0452.
Good, D. W. (2007). Nongenomic actions of aldosterone on the renal tubule. Hypertension, 49(4), 728-739.
Grossmann, C., & Gekle, M. (2009). New aspects of rapid aldosterone signaling. Molecular and cellular endocrinology, 308(1-2), 53-62.
Harris, A. N. et al. (2018). Mechanism of hyperkalemia-induced metabolic acidosis. J Am Soc Nephrol. 2018; 29(5):1411-1425.
Hartmann, R. et al. (2003). Discovery of selective CYP11B2 (aldosterone synthase) inhibitors for the therapy of congestive heart failure and myocardial fibrosis. Eur J Med Chem. 2003; 38:363-366.
Kem, David C., et al. “Circadian rhythm of plasma aldosterone concentration in patients with primary aldosteronism.” The Journal of clinical investigation 52.9 (1973): 2272-2277.
Kovesdy, C. P. (2017). Updates in hyperkalemia: Outcomes and therapeutic strategies. Rev Endocr Metab Disord. 2017; 18:41-7. doi:10.1007/s11154-016-9384-x.
Laragh, J. H. & Sealey J. E. (2011). The plasma renin test reveals the contribution of body sodium-volume content (V) and renin-angiotensin (R) vasoconstriction to long-term blood pressure. Am J Hypertens. 2011; 24(11):1164-80. doi: 10.1038/ajh.2011.171. Epub 2011 Sep. 22. PMID: 21938070.
Mihailidou, A. S., & Funder, J. W. (2005). Nongenomic effects of mineralocorticoid receptor activation in the cardiovascular system. Steroids, 70(5-7), 347-351.
Monticone, S. et al. (2017). Prevalence and clinical manifestations of primary aldosteronism encountered in primary care practice. J Am Coll Cardiol. 2017; 69:1811-20.
Nehme, A. et al. (2019). An Update on the Tissue Renin Angiotensin System and Its Role in Physiology and Pathology. J Cardiovasc Dev Dis. 2019; 6(2):14. doi: 10.3390/jcdd6020014. PMID: 30934934; PMCID: PMC6617132.
Noubiap J. J. et al. (2018). Global prevalence of resistant hypertension: a meta-analysis of data from 3.2 million patients. Heart. 2018; 105(2).
Parasiliti-Caprino, M. et al. (2020). Prevalence of primary aldosteronism and association with cardiovascular complications in patients with resistant and refractory hypertension. J Hypertens. 2020; 38:1841-8.
Pfizer (2020). INSPRA prescribing information. 2020. Available at www.pfizer.com
Schumacher, C. D. et al. (2013). Aldosterone synthase inhibition for the treatment of hypertension and the derived mechanistic requirements for a new therapeutic strategy. J Hypertens. 2013; 31:2085-93. doi: 10.1097/HJH.0b013e328363570c.
Strauch, B. et al. (2003). Prevalence of primary hyperaldosteronism in moderate to severe hypertension in the Central Europe region. J Hum Hypertens. 2003; 17:349-52.
Szylman, P. et al. (1976). Role of hyperkalemia in the metabolic acidosis of isolated hypoaldosteronism. N ENgl J Med. 1976; 294(7):361-365.
Trial on the Safety and Efficacy of Compound A in Patients with Uncontrolled Hypertension (Target-HTN). ClinicalTrials.gov identifier: NCT05001945. [updated 2021 Dec. 17; cited: 2021 20 Dec. 2021] Available at: clinicaltrials.gov
Williams, B. et al. (2015). Spironolactone versus placebo, bisoprolol, and doxazosin to determine the optimal treatment for drug-resistant hypertension (PATHWAY-2): a randomised, double-blind, crossover trial. Lancet. 2015; 386:2059-68.
Williams, G. H. (1972). Studies of the control of plasma aldosterone concentration in normal man: I. Response to posture, acute and chronic volume depletion, and sodium loading. The Journal of clinical investigation, 51(7), 1731-1742.
Yin, L. et al. (2012). 3-pyridyl substituted aliphatic cycles as CYP11B2 inhibitors: aromaticity abolishment of the core significantly increased selectivity over CYP1A2. PLoS One. 2012; 7:e48048.
Young, W. F. (2007). Primary aldosteronism: renaissance of a syndrome. Clin Endocrinol (Oxf). 2007; 66:607-18.

1. A method of treating hypertension in a hypertensive subject, the method comprising administering to the subject a CYP 11β2 beta hydroxylase inhibitor once or twice per day in an amount sufficient to inhibit 50% or more of CYP 11β2 beta hydroxylase's activity for 40-60% of a 24-hour period to thereby treat hypertension in the hypertensive subject, preferably wherein 50% or more of CYP 11β2 beta hydroxylase's activity is inhibited for between 10 to 14 hours of a 24-hour period.

2. (canceled)

3. A method of treating hypertension in a hypertensive subject, the method comprising
a) administering to the subject a CYP 11β2 beta hydroxylase inhibitor once or twice per day in an amount sufficient to reduce the serum aldosterone level of the subject by 50-90% relative to the subject's pre-drug level of serum aldosterone for a period not less than eight hours and not greater than 16 hours;
b) administering to the subject a CYP 11β2 beta hydroxylase inhibitor once per day in an amount sufficient to inhibit 50% or more of CYP 11β2 beta hydroxylase's activity for between 1 and 16 hours, preferably for between 3 and 8 hours; or
c) administering to the hypertensive subject a CYP 11β2 beta hydroxylase inhibitor once or twice per day in an amount sufficient to lower the hypertensive subject's ambulatory systolic blood pressure by at least 10 mmHg relative to the hypertensive subject's ambulatory systolic blood pressure prior to administration of the CYP 11β2 beta hydroxylase inhibitor.

4. The method of claim 3, wherein the CYP 11β2 beta hydroxylase inhibitor
a) reduces the serum aldosterone level of the subject by 60-80% relative to the subject's pre-drug level of serum aldosterone for a period not less than eight hours and not greater than 16 hours; and/or
b) allows serum aldosterone of the subject to return to the subject's pre-drug level of serum aldosterone or greater during the period between 16 and 24 hours after the dose is administered.

5. The method of claim 1, wherein the hypertensive subject is taking or has taken a hypertension medication selected from a diuretic, an ACE inhibitor, an angiotensin receptor blocker, a calcium channel blocker, or a combination of two or more thereof, preferably wherein the hypertensive subject is taking or has taken at least two of said hypertension medications.

6. The method of claim 1, wherein:
a) the CYP 11β2 beta hydroxylase inhibitor is administered to the subject once per day, preferably in the morning; or
b) the CYP 11β2 beta hydroxylase inhibitor is administered to the subject twice per day.
preferably wherein the CYP 11β2 beta hydroxylase inhibitor:
i) is administered daily for at least one week;
ii) is administered daily for at least two weeks;
iii) is administered daily for at least four weeks; or
iv) is administered daily for at least eight weeks.

7. (canceled)

8. The method of claim 1, wherein said CYP 11β2 beta hydroxylase inhibitor:
a) is selective for inhibition of CYP 11β2 beta hydroxylase activity relative to inhibition of CYP 11 β1 beta hydroxylase activity, preferably wherein the inhibition constant (Ki) for CYP 11 β1 beta hydroxylase divided by the Ki for CYP 11 β2 beta hydroxylase is greater than 100;
b) is administered to the hypertensive subject in an amount below the amount which causes the subject's serum and/or plasma 11-deoxycortisterone (11-DOC) levels to exceed 600 pmol/L, preferably below the amount which causes the subject's serum and/or plasma 11-DOC levels to exceed 400 pmol/L;
c) is administered to the hypertensive subject in an amount below the amount which causes an accumulation of 11-DOC above 0.1 ng/ml in the subject;
d) is administered to the hypertensive subject in an amount which does not cause a clinically meaningful upregulation of the subject's adrenocortical hormone synthesis;
e) is administered to the hypertensive subject in an amount which:
i) does not cause a clinically meaningful reduction of the subject's serum and/or plasma cortisol levels, relative to the subject's serum and/or plasma cortisol levels prior to administration of the CYP 11β2 beta hydroxylase inhibitor;
ii) does not cause a clinically meaningful increase in the subject's serum and/or plasma 11-DOC levels relative to the subject's serum and/or plasma 11-DOC levels prior to administration of the CYP 11β2 beta hydroxylase inhibitor; and/or
iii) does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; and/or
f) is administered to the hypertensive subject in an amount:
i) which does not cause a reduction of more than 20% in the subject's serum and/or plasma cortisol levels, relative to the subject's serum and/or plasma cortisol levels prior to administration of the CYP 11β2 beta hydroxylase inhibitor, preferably which does not cause a reduction of more than 10% in the subject's serum and/or plasma cortisol levels, relative to the subject's serum and/or plasma cortisol levels prior to administration of the CYP 11β2 beta hydroxylase inhibitor;
ii) which does not cause an increase of more than 20% in the subject's serum and/or plasma 11-DOC levels relative to the subject's serum and/or plasma 11-DOC levels prior to administration of the CYP 11β2 beta hydroxylase inhibitor, preferably which does not cause an increase of more than 10% in the subject's serum and/or plasma 11-DOC levels relative to the subject's serum and/or plasma 11-DOC levels prior to administration of the CYP 11β2 beta hydroxylase inhibitor; and/or
iii) which does not cause an increase of more than 20% in the subject's serum and/or plasma 11-deoxycortisol levels relative to the subject's serum and/or plasma 11-deoxycortisol levels prior to administration of the CYP 11β2 beta hydroxylase inhibitor, preferably which does not cause an increase of more than 10% in the subject's serum and/or plasma 11-deoxycortisol levels relative to the subject's serum and/or plasma 11-deoxycortisol levels prior to administration of the CYP 11β2 beta hydroxylase inhibitor.

9. The method of claim 1, wherein said CYP 11β2 beta hydroxylase inhibitor is a compound of Formula (A) or a pharmaceutically acceptable salt thereof: 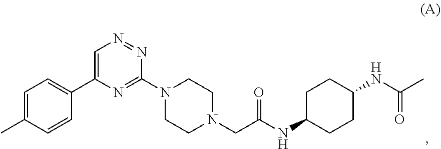 [SMILES: CC(=O)N[C@H]1CC[C@H](NC(=O)CN2CCN(c3nncc(-c4ccc(C)cc4)n3)CC2)CC1]
preferably wherein the compound is in the form of an HBr salt of the compound of Formula (A).

10. The method of claim 9, wherein:
a) between 5 mg and 100 mg of the CYP 11β2 beta hydroxylase inhibitor is administered orally twice a day, 12 hours apart;
b) between 10 mg and 50 mg of the CYP 11β2 beta hydroxylase inhibitor is administered orally twice a day, 12 hours apart;
c) between 5 mg and 100 mg of the CYP 11β2 beta hydroxylase inhibitor is administered orally once a day; or
d) between 10 mg and 50 mg of the CYP 11β2 beta hydroxylase inhibitor is administered orally once a day;
preferably wherein:
e) 12.5 mg of the CYP 11β2 beta hydroxylase inhibitor is administered orally twice a day, 12 hours apart;
f) 25 mg of the CYP 11β2 beta hydroxylase inhibitor is administered orally twice a day, 12 hours apart;
g) 12.5 mg of the CYP 11β2 beta hydroxylase inhibitor is administered orally once a day;
h) 50 mg of the CYP 11β2 beta hydroxylase inhibitor is administered orally once a day; or
i) 100 mg of the CYP 11β2 beta hydroxylase inhibitor is administered orally once a day.

11. (canceled)

12. The method of claim 1, wherein:
a) the subject's office-measured systolic blood pressure is lowered, preferably by at least 10 mmHg, relative to the subject's office-measured systolic blood pressure prior to administration of the CYP 11β2 beta hydroxylase inhibitor;
b) the subject's 24-hour ambulatory systolic blood pressure is lowered, preferably by at least 10 mmHg, relative to the subject's ambulatory systolic blood pressure prior to administration of the CYP 11β2 beta hydroxylase inhibitor;
c) the subject's office-measured diastolic blood pressure is lowered relative to the subject's office-measured diastolic blood pressure prior to administration of the CYP 11β2 beta hydroxylase inhibitor;
d) the subject's office-measured systolic and diastolic blood pressure is lowered relative to the subject's office-measured systolic and diastolic blood pressure prior to administration of the CYP 11β2 beta hydroxylase inhibitor, preferably wherein the subject's office-measured systolic blood pressure is lowered by at least 10 mmHg and the subject's office-measured diastolic blood pressure is lowered by at least 5 mmHg, each relative to the subject's office-measured systolic and diastolic blood pressure, respectively, prior to administration of the CYP 11β2 beta hydroxylase inhibitor;
e) the subject's ambulatory systolic and diastolic blood pressure are lowered relative to the subject's ambulatory systolic and diastolic blood pressure prior to administration of the CYP 11β2 beta hydroxylase inhibitor, preferably wherein the subject's ambulatory systolic blood pressure is lowered by at least 10 mmHg, and the subject's ambulatory diastolic blood pressure is lowered by at least 5 mmHg, each relative to the subject's ambulatory systolic and diastolic blood pressure, respectively, prior to administration of the CYP 11β2 beta hydroxylase inhibitor;
f) the subject's systolic blood pressure is reduced to less than 130 mmHg and/or the subject's diastolic blood pressure is reduced to less than 80 mmHg.

13. (canceled)

14. (canceled)

15. (canceled)

16. The method of claim 1, wherein the hypertensive subject's average systolic blood pressure during sleep is reduced
a) relative to the hypertensive subject's average systolic blood pressure during sleep prior to receiving the CYP 11β2 beta hydroxylase inhibitor, and/or
b) relative to the hypertensive subject's average daytime systolic blood pressure;
preferably wherein the hypertensive subject's average systolic blood pressure during sleep is reduced:
i) by at least 10%, by between 10% and 40%, by between 10% and 30%, or by between 10% and 20% relative to the hypertensive subject's average daytime systolic blood pressure; and/or
ii) by at least 8 mmHg, by at least 10 mmHg, by between 8 and 55 mmHg, by between 10 and 45 mmHg, or by between 10 and 25 mmHg, relative to the hypertensive subject's average systolic blood pressure during sleep prior to receiving the CYP 11β2 beta hydroxylase inhibitor.

17. The method of claim 1, wherein:
a) the duration of inhibition of CYP 11β2 beta hydroxylase activity is sufficient to maintain a state of sodium and volume depletion in the hypertensive subject;
b) the method does not produce a persistent state of hyperkalemia or mild non-anion gap metabolic acidosis in the hypertensive subject;
c) the CYP 11β2 beta hydroxylase inhibitor does not substantially accumulate in the hypertensive subject, preferably wherein the lack of substantial accumulation of the CYP 11β2 beta hydroxylase inhibitor in the hypertensive subject allows for the hypertensive subject's aldosterone levels to return to pre-drug baseline within 24-48 hours of the CYP 11β2 beta hydroxylase inhibitor being administered, more preferably within 16-24 hours of the CYP 11β2 beta hydroxylase inhibitor being administered;
d) the hypertensive subject's potassium levels are generally maintained in a clinically normal range, preferably wherein the hypertensive subject's potassium levels are mildly elevated relative to the hypertensive subject's potassium levels prior to administration of the CYP 11β2 beta hydroxylase inhibitor, more preferably wherein the hypertensive subject's potassium levels are elevated by 0.35 mmol/L or less, more preferably wherein the hypertensive subject's potassium levels are maintained below a level of 5.5 mmol/L, more preferably wherein the hypertensive subject's potassium levels are maintained between 3.5 mEq/l to 5.1 mEq/l; and/or
e) the CYP 11β2 beta hydroxylase inhibitor is administered to the hypertensive subject in an amount which:
i) suppresses aldosterone production in the subject;
ii) increases serum and/or plasma potassium levels in the subject; and/or
iii) increases plasma renin activity (PRA) in the subject;
preferably wherein:
iv) serum and/or plasma aldosterone AUC-24 is reduced in the subject by at least 25% relative to the aldosterone levels in the subject prior to administration of the CYP 11β2 beta hydroxylase inhibitor;
v) serum and/or plasma potassium levels in the subject are increased by at least 0.2 mMol/L relative to the serum and/or plasma potassium levels in the subject prior to administration of the CYP 11β2 beta hydroxylase inhibitor; and/or
vi) PRA in the subject is increased by at least 5 ng/ml/hr relative to the PRA in the subject prior to administration of the CYP 11β2 beta hydroxylase inhibitor;
more preferably wherein the hypertensive subject's aldosterone level follows a substantially normal circadian rhythm.

18. (canceled)

19. (canceled)

20. (canceled)

21. A method of reducing a hypertensive subject's systolic blood pressure during sleep, the method comprising administering to the subject a CYP 11β2 beta hydroxylase inhibitor once or twice per day in an amount sufficient to reduce the hypertensive subject's average systolic blood pressure during sleep (a) relative to the hypertensive subject's average systolic blood pressure during sleep prior to receiving the CYP 11β2 beta hydroxylase inhibitor, and/or (b) relative to the hypertensive subject's average daytime systolic blood pressure, preferably wherein the hypertensive subject's average systolic blood pressure during sleep is reduced:
a) by at least 10%, by between 10% and 40%, by between 10% and 30%, or by between 10% and 20% relative to the hypertensive subject's average daytime systolic blood pressure; and/or
b) by at least 8 mmHg, by at least 10 mmHg, by between 8 and 55 mmHg, by between 10 and 45 mmHg, or by between 10 and 25 mmHg, relative to their average systolic blood pressure during sleep prior to receiving the CYP 11β2 beta hydroxylase inhibitor.

22. (canceled)

23. The method of claim 21, wherein the hypertensive subject is taking or has taken a hypertension medication selected from a diuretic, an ACE inhibitor, an angiotensin receptor blocker, a calcium channel blocker, or a combination of two or more thereof, preferably wherein the hypertensive subject is taking or has taken at least two of said hypertension medications.

24. The method of claim 21, wherein:
a) 50% or more of CYP 11β2 beta hydroxylase's activity is inhibited for 40-60% of a 24-hour period;
b) 50% or more of CYP 11β2 beta hydroxylase's activity is inhibited for between 10 to 14 hours of a 24-hour period;
c) the CYP 11β2 beta hydroxylase inhibitor reduces the serum aldosterone level of the subject by 50-90% relative to the subject's pre-drug level of serum aldosterone for a period not less than eight hours and not greater than 16 hours;
d) the CYP 11β2 beta hydroxylase inhibitor reduces the serum aldosterone level of the subject by 60-80% relative to the subject's pre-drug level of serum aldosterone for a period not less than eight hours and not greater than 16 hours;
e) the CYP 11β2 beta hydroxylase inhibitor allows serum aldosterone of the subject to return to the subject's pre-drug level of serum aldosterone or greater during the period between 16 and 24 hours after the dose is administered; and/or
f) 50% or more of CYP 11β2 beta hydroxylase's activity is inhibited for between 1 and 16 hours, or preferably for between 3 and 8 hours, of a 24-hour period.

25. The method of claim 21, wherein:
a) the CYP 11β2 beta hydroxylase inhibitor is administered to the subject once per day, preferably in the morning; or
b) the CYP 11β2 beta hydroxylase inhibitor is administered to the subject twice per day;
preferably wherein the CYP 11β2 beta hydroxylase inhibitor:
i) is administered daily for at least one week;
ii) is administered daily for at least two weeks;
iii) is administered daily for at least four weeks; or
iv) is administered daily for at least eight weeks.

26. (canceled)

27. The method of claim 21, wherein:
a) the hypertensive subject's ambulatory systolic blood pressure is reduced by 10-55 mmHg, by 10-50 mmHg, by 10-45 mmHg, by 10-40 mmHg, by 10-35 mmHg, by 10-30 mmHg, by 10-25 mmHg, by 10-20 mmHg, or by 10-15 mmHg, relative to the hypertensive subject's ambulatory systolic blood pressure prior to administration of the CYP 11β2 beta hydroxylase inhibitor for a period of at least eight weeks;
b) the hypertensive subject's ambulatory diastolic blood pressure is reduced by 5-25 mmHg, by 5-20 mmHg, or by 5-15 mmHg relative to the hypertensive subject's ambulatory diastolic blood pressure prior to administration of the CYP 11β2 beta hydroxylase inhibitor for a period of at least eight weeks;
c) the duration of inhibition of CYP 11β2 beta hydroxylase activity is sufficient to maintain a state of sodium and volume depletion in the hypertensive subject;
d) the method does not produce a persistent state of hyperkalemia or mild non-anion gap metabolic acidosis in the hypertensive subject;
e) the CYP 11β2 beta hydroxylase inhibitor does not substantially accumulate in the hypertensive subject, preferably wherein the lack of substantial accumulation of the CYP 11β2 beta hydroxylase inhibitor in the hypertensive subject allows for the hypertensive subject's aldosterone levels to return to pre-drug baseline within 24-48 hours of the CYP 11β2 beta hydroxylase inhibitor being administered, more preferably within 16-24 hours of the CYP 11β2 beta hydroxylase inhibitor being administered;
f) the hypertensive subject's potassium levels are generally maintained in a clinically normal range, preferably wherein the hypertensive subject's potassium levels are mildly elevated relative to the hypertensive subject's potassium levels prior to administration of the CYP 11β2 beta hydroxylase inhibitor, more preferably wherein the hypertensive subject's potassium levels are elevated by 0.35 mmol/L or less, more preferably wherein the hypertensive subject's potassium levels are maintained below a level of 5.5 mmol/L, more preferably wherein the hypertensive subject's potassium levels are maintained between 3.5 mEq/l to 5.1 mEq/l; and/or
g) the CYP 11β2 beta hydroxylase inhibitor is administered to the hypertensive subject in an amount which:
i) suppresses aldosterone production in the subject;
ii) increases serum and/or plasma potassium levels in the subject; and/or
iii) increases plasma renin activity (PRA) in the subject;
preferably wherein:
iv) serum and/or plasma aldosterone AUC-24 is reduced in the subject by at least 25% relative to the aldosterone levels in the subject prior to administration of the CYP 11β2 beta hydroxylase inhibitor;
v) serum and/or plasma potassium levels in the subject are increased by at least 0.2 mMol/L relative to the serum and/or plasma potassium levels in the subject prior to administration of the CYP 11β2 beta hydroxylase inhibitor; and/or
vi) PRA in the subject is increased by at least 5 ng/ml/hr relative to the PRA in the subject prior to administration of the CYP 11β2 beta hydroxylase inhibitor;
more preferably wherein the hypertensive subject's aldosterone level follows a substantially normal circadian rhythm.

28. (canceled)

29. (canceled)

30. The method of claim 21, wherein said CYP 11β2 beta hydroxylase inhibitor
a) is selective for inhibition of CYP 11β2 beta hydroxylase activity relative to inhibition of CYP 11β1 beta hydroxylase activity, preferably wherein the inhibition constant (Ki) for CYP 11β1 beta hydroxylase divided by the Ki for CYP 11β2 beta hydroxylase is greater than 100;
b) is administered to the hypertensive subject in an amount below the amount which causes the subject's serum and/or plasma 11-deoxycortisterone (11-DOC) levels to exceed 600 pmol/L, preferably below the amount which causes the subject's serum and/or plasma 11-DOC levels to exceed 400 pmol/L;
c) is administered to the hypertensive subject in an amount below the amount which causes an accumulation of 11-DOC above 0.1 ng/ml in the subject;
d) is administered to the hypertensive subject in an amount which does not cause a clinically meaningful upregulation of the subject's adrenocortical hormone synthesis;
e) is administered to the hypertensive subject in an amount which:
i) does not cause a clinically meaningful reduction of the subject's serum and/or plasma cortisol levels, relative to the subject's serum and/or plasma cortisol levels prior to administration of the CYP 11β2 beta hydroxylase inhibitor;
ii) does not cause a clinically meaningful increase in the subject's serum and/or plasma 11-DOC levels relative to the subject's serum and/or plasma 11-DOC levels prior to administration of the CYP 11β2 beta hydroxylase inhibitor; and/or
iii) does not cause a clinically meaningful increase in the subject's serum and/or plasma 11-deoxycortisol levels relative to the subject's serum and/or plasma 11-deoxycortisol levels prior to administration of the CYP 11β2 beta hydroxylase inhibitor; and/or
f) is administered to the hypertensive subject in an amount:
i) which does not cause a reduction of more than 20% in the subject's serum and/or plasma cortisol levels, relative to the subject's serum and/or plasma cortisol levels prior to administration of the CYP 11β2 beta hydroxylase inhibitor, preferably which does not cause a reduction of more than 10% in the subject's serum and/or plasma cortisol levels, relative to the subject's serum and/or plasma cortisol levels prior to administration of the CYP 11β2 beta hydroxylase inhibitor;
ii) which does not cause an increase of more than 20% in the subject's serum and/or plasma 11-DOC levels relative to the subject's serum and/or plasma 11-DOC levels prior to administration of the CYP 11β2 beta hydroxylase inhibitor, preferably which does not cause an increase of more than 10% in the subject's serum and/or plasma 11-DOC levels relative to the subject's serum and/or plasma 11-DOC levels prior to administration of the CYP 11β2 beta hydroxylase inhibitor; and/or
iii) which does not cause an increase of more than 20% in the subject's serum and/or plasma 11-deoxycortisol levels relative to the subject's serum and/or plasma 11-deoxycortisol levels prior to administration of the CYP 11β2 beta hydroxylase inhibitor, preferably which does not cause an increase of more than 10% in the subject's serum and/or plasma 11-deoxycortisol levels relative to the subject's serum and/or plasma 11-deoxycortisol levels prior to administration of the CYP 11β2 beta hydroxylase inhibitor.

31. The method of claim 21, wherein said CYP 11β2 beta hydroxylase inhibitor is a compound of Formula (A) or a pharmaceutically acceptable salt thereof: 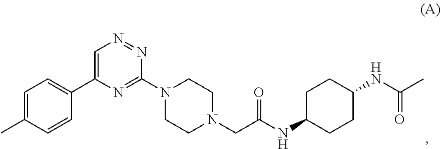 [SMILES: CC(=O)N[C@H]1CC[C@H](NC(=O)CN2CCN(c3nncc(-c4ccc(C)cc4)n3)CC2)CC1]
preferably wherein the compound is in the form of an HBr salt of the compound of Formula (A).

32. The method of claim 31, wherein:
a) between 5 mg and 100 mg of the CYP 11β2 beta hydroxylase inhibitor is administered orally twice a day, 12 hours apart;
b) between 10 mg and 50 mg of the CYP 11β2 beta hydroxylase inhibitor is administered orally twice a day, 12 hours apart;
c) between 5 mg and 100 mg of the CYP 11β2 beta hydroxylase inhibitor is administered orally once a day; or
d) between 10 mg and 50 mg of the CYP 11β2 beta hydroxylase inhibitor is administered orally once a day;
preferably wherein:
e) 12.5 mg of the CYP 11β2 beta hydroxylase inhibitor is administered orally twice a day, 12 hours apart;
f) 25 mg of the CYP 11β2 beta hydroxylase inhibitor is administered orally twice a day, 12 hours apart;
g) 12.5 mg of the CYP 11β2 beta hydroxylase inhibitor is administered orally once a day;
h) 50 mg of the CYP 11β2 beta hydroxylase inhibitor is administered orally once a day; or
i) 100 mg of the CYP 11β2 beta hydroxylase inhibitor is administered orally once a day.

33. (canceled)

34. (canceled)

35. The method of claim 1, wherein the hypertensive subject:
a) has secondary hypertension, preferably primary aldosteronism; or
b) does not have primary aldosteronism, preferably wherein the hypertensive subject has primary hypertension.

36. (canceled)
